(19) United States
(12) Patent Application Publication
(10) Pub. No.: US 2006/0111305 A1
(43) Pub. Date: May 25, 2006

(54) Metabolites of certain [1,4]diazepino[6,7,1-ij]quinoline derivatives and methods of preparation and use thereof

(75) Inventors: Zeen Tong, Collegeville, PA (US); Jim Wang, Malvern, PA (US); William DeMaio, Collegeville, PA (US); Alvin C. Bach II, Riverton, NJ (US); Ronald A. Jordan, Richboro, PA (US); Youchu Wang, Sainte-Anne-de-Bellevue (CA); P. Sivaramakrishnan Ramamoorthy, Plainsboro, NJ (US); Gary P. Stack, Ambler, PA (US)

Correspondence Address: COZEN O' CONNOR, P. C., 1900 MARKET STREET, PHILADELPHIA, PA, 19103-3508, US

(73) Assignee: Wyeth, Madison, NJ (US)

(21) Appl. No.: 11/267,765

(22) Filed: Nov. 4, 2005

Related U.S. Application Data
(60) Provisional application No. 60/625,335, filed on Nov. 5, 2004.

(51) Int. Cl. A61K 31/7052; C07H 19/00; C07H 19/22; C07H 17/02

(52) U.S. Cl. 514/23; 514/43; 536/17.4; 536/27.1

(57) ABSTRACT

The invention relates to metabolites of certain [1,4]diazepino[6,7,1-ij]quinoline derivatives and methods of preparation and use thereof. Specifically, the invention relates to compounds of formula I 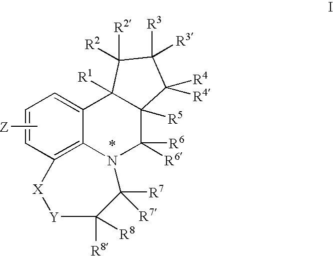 wherein the various substituents are defined herein. The invention also provides pharmaceutical compositions including compounds of formula I, methods of making such compounds, and methods of using such compounds.

This application claims benefit of priority to U.S. provisional patent application Ser. No. 60/625,335 filed Nov. 5, 2004, which is hereby incorporated by reference in its entirety.

### FIELD OF THE INVENTION

The present invention relates to metabolites of certain [1,4]diazepino[6,7,1-ij]quinoline derivatives, which are useful as antipsychotic and antiobesity agents, to processes for their preparation, to pharmaceutical compositions containing them, and to methods of using them.

### BACKGROUND OF THE INVENTION

Schizophrenia affects approximately 5 million people. At present, the most widespread treatments for schizophrenia are the ‘atypical’ antipsychotics, which combine dopamine (D2) receptor antagonism with serotonin (5-HT2A) receptor antagonism. Despite the reported advances in efficacy and side-effect liability of atypical antipsychotics over typical antipsychotics, these compounds do not adequately treat all of the symptoms of schizophrenia and are accompanied by problematic side effects including weight gain (Allison, D. B., et. al., Am. J. Psychiatry, 156: 1686-1696, 1999; Masand, P. S., Exp. Opin. Pharmacother. I: 377-389, 2000; Whitaker, R., Spectrum Life Sciences. Decision Resources. 2:1-9, 2000). Novel antipsychotics which are effective in treating the mood disorders or the cognitive impairments in schizophrenia without producing weight gain would represent a significant advance in the treatment of schizophrenia.

5-HT_{2C} agonists and partial agonists represent a novel therapeutic approach toward the treatment of schizophrenia. Several lines of evidence support a role for 5-HT_{2C} receptor agonism as a treatment for schizophrenia. Studies with 5-HT_{2C} antagonists suggest that these compounds increase synaptic levels of dopamine and may be effective in animal models of Parkinson's disease (Di Matteo, V., et. al., Neuropharmacology 37: 265-272, 1998; Fox, S. H., et. al., Experimental Neurology 151: 35-49, 1998). Since the positive symptoms of schizophrenia are associated with increased levels of dopamine, compounds with actions opposite those of 5-HT_{2C} antagonists such as 5-HT_{2C} agonists and partial agonists should reduce levels of synaptic dopamine. Recent studies have demonstrated that 5-HT_{2C} agonists decrease levels of dopamine in the prefrontal cortex and nucleus accumbens (Millan, M. J., et. al., Neuropharmacology 37: 953-955,1998; Di Matteo, V., et. al., Neuropharmacology 38: 1195-1205, 1999; Di Giovanni, G., et. al., Synapse 35: 53-61, 2000), brain regions that are thought to mediate critical antipsychotic effects of drugs like clozapine. In contrast, 5-HT_{2C} agonists do not decrease dopamine levels in the striatum, the brain region most closely associated with extrapyramidal side effects. In addition, a recent study demonstrates that 5-HT_{2C} agonists decrease firing in the ventral tegmental area (VTA), but not in substantia nigra. The differential effects of 5-HT_{2C} agonists in the mesolimbic pathway relative to the nigrostriatal pathway suggests that 5-HT_{2C} agonists will have limbic selectivity and will be less likely to produce extrapyramidal side effects associated with typical antipsychotics.

Atypical antipsychotics bind with high affinity to 5-HT_{2C} receptors and function as 5-HT_{2C} receptor antagonists or inverse agonists. Weight gain is a problematic side effect associated with atypical antipsychotics such as clozapine and olanzapine and it has been suggested that 5-HT_{2C} antagonism is responsible for the increased weight gain. Conversely, stimulation of the 5-HT_{2C} receptor is known to result in decreased food intake and body weight (Walsh et. al., Psychopharmacology 124: 57-73, 1996; Cowen, P. J., et. al., Human Psychopharmacology 10: 385-391, 1995; Rosenzweig-Lipson, S., et. al., ASPET abstract, 2000). As a result, 5-HT_{2C} agonists and partial agonists will be less likely to produce the body weight increases associated with current atypical antipsychotics. Indeed, 5-HT_{2C} agonists and partial agonists are of great interest for the treatment of obesity, a medical disorder characterized by an excess of body fat or adipose tissue and associated with such comorbidities as Type II diabetes, cardiovascular disease, hypertension, hyperlipidemia, stroke, osteoarthritis, sleep apnea, gall bladder disease, gout, some cancers, some infertility, and early mortality.

The compound (9aR,12aS)-4,5,6,7,9,9a,10,11,12,12a-decahydro-cyclopenta[c][1,4]diazepine[6,7,1-ij]quinoline (hereafter DCDQ): 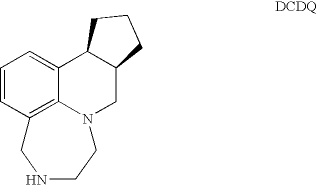
is a potent 5-HT_{2C} agonist. See related published applications WO03/091250 and US2004/0009970, each of which is incorporated by reference herein in its entirety. DCDQ can also be effective in treating the mood disorders or the cognitive impairments associated with schizophrenia. DCDQ is converted, in several in vitro and in vivo models, into several metabolites. It can be seen that these metabolites are of interest in treating those diseases, disorders, or conditions treatable by DCDQ, itself as is or as a prodrug which converts to DCDQ. These metabolites could also be useful for further studying the effects of DCDQ. This invention is directed to these, as well as other, important ends.

### SUMMARY OF THE INVENTION

Some embodiments of the invention include compounds formula I 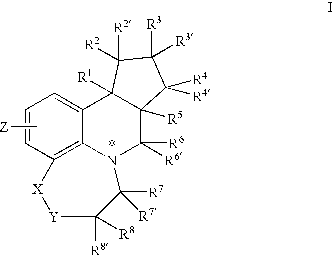
wherein:
for each Rⁿ and R^{n′}, where n is 1 through 8:

each Rⁿ and R^{n′} is independently hydrogen, hydroxy, CH₃C(O)—O—, —OSO₃H, or —O-G; or

Rⁿ and the corresponding R^{n′}, where n is 2, 3, 4, 6, 7, or 8, taken together with the carbon to which they are attached, form C═O; or

Rⁿ along with the corresponding Rⁿ⁺¹, where n is 1, 2, 3, 4, 5, or 7, taken together form a double bond between the carbons to which they are attached, and each corresponding R^{n′} and R^{(n+1)′} is independently hydrogen, hydroxy, CH₃C(O)—O, —OSO₃H, or —O-G;

G has the formula:

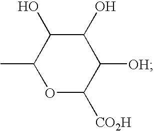

wherein the nitrogen denoted with the symbol * can optionally form an N-oxide;

X—Y is CH═N, CH═N(O), CH₂N(O), C(O)NH or CR⁹HNR¹⁰;

R⁹ is hydrogen, hydroxyl, or —OSO₃H;

R¹⁰ is hydrogen, acetyl, —SO₃H, -G, or —C(O)—OG;

Z is hydrogen, hydroxy, —OSO₃H, or —O-G;

with the proviso that when Z is hydroxy, then either (a) one of R¹, R², R³, R⁴, R⁵, R⁶, R⁷, R⁸, R⁹, and R¹⁰ is not hydrogen; or (b) X—Y is not CR⁹HNR¹⁰; and

with the further proviso that when X—Y is CHR⁹NR¹⁰, then at least one of Z, R¹, R², R³, R⁴, R⁵, R⁶, R⁷, R⁸, R⁹, and R¹⁰ is not H.

In some embodiments, the invention provides compounds according to Formula I, wherein at least one of Z and R¹ through R⁸ is —OH.

In some embodiments, the invention provides compounds according to Formula I wherein at least one of R¹ through R⁶, R⁹, R¹⁰, and Z is —C(O)—O-G, —O-G, or -G.

In some embodiments, the invention provides compounds according to Formula I, wherein at least one of R¹ through R⁹, and Z is —OSO₃H.

In some embodiments, the invention provides compounds according to Formula I, wherein X—Y is CR⁹HNR¹⁰, where R⁹ is H and R¹⁰ is —SO₃H.

In some embodiments, the invention provides compounds according to Formula I, wherein Rⁿ and corresponding R^{n′} taken together with the carbon to which they are attached form C═O.

In some embodiments, the invention provides compounds according to Formula I, wherein X—Y is C(O)NH.

In some embodiments, the invention provides compounds according to Formula I, wherein X—Y is CH═N.

In some embodiments, the invention provides compounds according to Formula I, wherein at least one of Rⁿ and its corresponding Rⁿ⁺¹, where n=1-5, together form a double bond between the carbons to which they are attached and each R^{n′} and R^{(n+1)′} is independently hydrogen, hydroxy, CH₃C(O)—O, —OSO₃H, or —O-G.

In some embodiments, the invention provides isolated or substantially pure forms of compounds of Formula I, having at least 75% purity. In other embodiments, the invention provides compounds of Formula I having at least 80% purity. In other embodiments, the invention provides compounds of Formula I having at least 85% purity. In other embodiments, the invention provides compounds of Formula I having at least 90% purity. In other embodiments, the invention provides compounds of Formula I having at least 95% purity.

In some embodiments, the invention provides pharmaceutical compositions including compounds of formula I.

In some embodiments, the invention provides methods of treating conditions, diseases, or disorders associated with 5HT_{2C} by administering compounds of Formula I or pharmaceutical compositions comprising compounds of Formula I to a patient in need thereof.

In some embodiments, the invention provides a method of preparing a compound of formula M6: 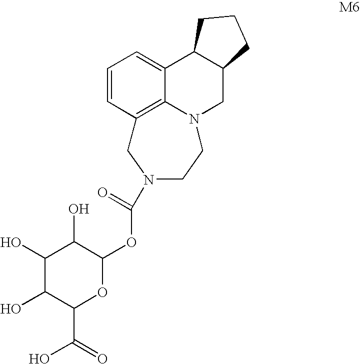
comprising:

reacting Compound 6a:

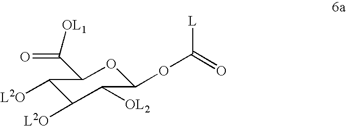

where each L, L¹, and L² is a leaving group;

with DCDQ: 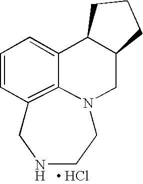
in the presence of a coupling reagent under conditions sufficient to yield compound 7: 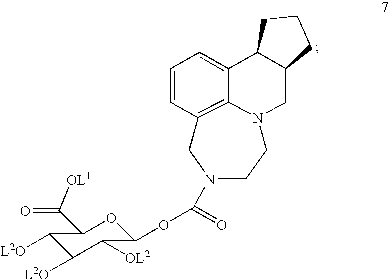
   and removing said leaving groups L¹ and L² to form compound M6.

In some embodiments, the invention provides methods wherein L has the formula:

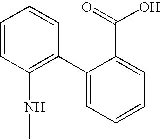

In some embodiments, the invention provides methods wherein L¹ and L² are independently selected from lower alkyl and acetyl, such as when L¹ is methyl and each L² is acetyl.

Preferred coupling reagents are (Benzotriazol-1-yloxy)tris(dimethylamino)phosphonium hexafluorophosphate (BOP), N,N′-Dicyclohexylcarbodiimide (DCC), and 1-(3-dimethylaminopropyl)-3-ethylcarbodiimide hydrochloride (EDC).

In some embodiments, the invention provides a method further comprising deprotecting compound 7 by removing the L¹ and L² protecting groups of the glucuronyl moiety of compound 7, thereby forming the M6 metabolite. In some embodiments, the deprotecting step is performed in alcohol, preferably a lower alkyl alcohol, in the presence of a base, preferably NaOH, LiOH, or KOH. In some preferred embodiments, LiOH.H₂O in MeOH/H₂O/THF is used in a preferred ratio of approximately 2.5:1.0:0.5. In some embodiments, the deprotection reaction is carried out at 0° C. for 1 hour.

In some embodiments, the reaction of compound 6 with the coupling reagent and DCDQ is carried out in the presence of an amine, preferably Hünig's base. This reaction is preferably performed in a solvent, such as CH₂Cl₂.

In some embodiments, compound 7 is subjected to column chromatography purification prior to deprotection.

In some embodiments, the invention further provides for purifying the M6 metabolite.

In some embodiments, the invention provides methods where compound 6a is prepared by removing the allyl protecting group of compound 5: 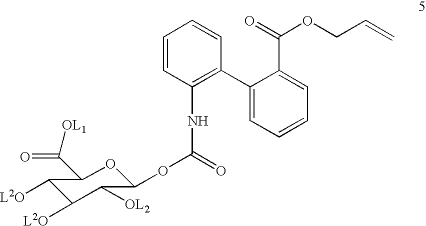
using a catalyst and a nucleophile, preferably morpholine. In some embodiments, the catalyst is Pd(PPh₃)₄.

In some embodiments, the invention provides methods wherein compound 5 is prepared by reacting carboxylic acid 2: 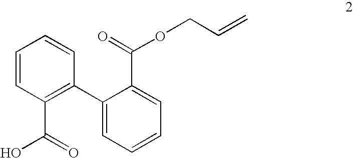
with DPPA under conditions sufficient to yield an acyl azide intermediate;

heating resultant acyl azide intermediate under conditions sufficient to yield isocyanate 3:

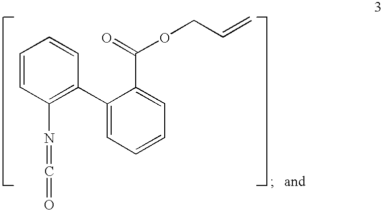

treating result of said heating step with 2,3,4,-triacetyl-1-hydroxyglucoronic ester 4: 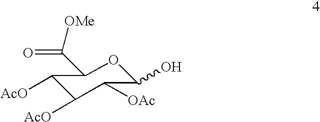
under conditions sufficient to yield compound 5. In some embodiments, the reacting step is carried out in the presence of a base, preferably Et₃N.

In some embodiments, the invention provides methods wherein compound 2 is prepared by reacting diphenic anhydride with excess allyl alcohol, preferably prop-2-en-1-ol, in the presence of a catalyst, preferably DMAP.

These and other embodiments of the invention will be apparent to those of skill in the art upon reading this disclosure.

### BRIEF DESCRIPTION OF THE DRAWINGS

FIG. 1 is a flowchart of proposed metabolic pathways of DCDQ identified in the in vitro and in vivo studies.

FIG. 2 is a further flowchart of proposed metabolic pathways of DCDQ identified in rat biliary excretion studies.

FIG. 3 is a further flowchart of proposed metabolic pathways of DCDQ identified in mice.

FIG. 4 is a further flowchart of proposed metabolic pathways of DCDQ identified in human plasma.

FIG. 5 depicts structures and NMR numbering schemes for DCDQ, M7, M9 and M13 as identified in the rat billiary excretion studies.

### DETAILED DESCRIPTION OF THE INVENTION

In some aspects, this invention relates to metabolites of DCDQ, methods of preparing them, and methods of using them to treat various disorders.

In some aspects, the present invention provides compounds of formula (I) 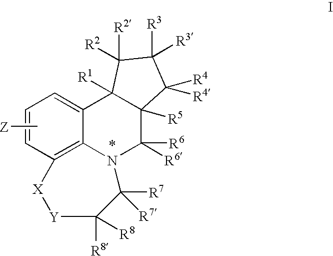
wherein:

for each Rⁿ and R^{n′} where n is 1 through 8:
each Rⁿ and R^{n′} is independently hydrogen, hydroxy, CH₃C(O)—O, —OSO₃H, or —O-G; or

Rⁿ and the corresponding R^{n′}, where n is 2, 3, 4, 6, 7, or 8, taken together with the carbon to which they are attached, form C═O; or

Rⁿ along with the corresponding Rⁿ⁺¹, where n is 1, 2, 3, 4, 5, or 7, taken together form a double bond between the carbons to which they are attached, and each corresponding R^{n′} and R^{(n+1)′} is independently hydrogen, hydroxy, CH₃C(O)—O, —OSO₃H, or —O-G;

wherein the nitrogen denoted with the symbol * can optionally form an N-oxide;

G has the formula:

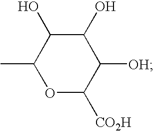

X—Y is CH═N, CH═N(O), CH₂N(O), C(O)NH or CR⁹HNR¹⁰;

R⁹ is hydrogen, hydroxyl, or —OSO₃H; and

R¹⁰ is hydrogen, acetyl, —SO₃H, -G. or —C(O)—OG;

Z is hydrogen, hydroxy, —OSO₃H, or —O-G, with the proviso that when Z is hydroxy, then either (a) one of R¹, R², R³, R⁴, R⁵, R⁶, R⁷, R⁸, R⁹, and R¹⁰ is not hydrogen; or (b) X—Y is not CR⁹HNR¹⁰;

with the further proviso that when X—Y is CHR⁹NR¹⁰, then at least one of Z, R¹, R², R³, R⁴, R⁵, R⁶, R⁷, R⁸, R⁹, and R¹⁰ is not H.

Methods for the preparation of DCDQ (i.e., the compound of Formula I where X—Y is CHR⁹NR¹⁰ and each of Z, and R¹ through R¹⁰ is H) is disclosed in U.S. Patent Application Publication No. US2004/0009970, hereby incorporated by reference herein in its entirety. DCDQ itself is not intended to be within the compounds of Formula I disclosed herein.

In some embodiments, the invention provides hydroxy compounds of formula I. Preferably, at least one of Z and R¹ through R⁸ is hydroxy.

In some embodiments, the invention further provides hydroxy compounds of formula I where X—Y is CR⁹HNR¹⁰. Some examples of such hydroxy compounds include those where:

R⁹ and R¹⁰ are each H;

at least one of R⁷ and R⁸ is —OH;

R⁶ is —OH;

at least one of R³ and R⁴ is —OH; or

at least one of R¹, R⁵, R⁶, R⁷, and Z is —OH.

In other embodiments, the invention provides hydroxy compounds of formula I where X—Y is CR⁹HNR¹⁰ and R¹⁰ is acetyl. Some preferred examples of such hydroxyl compounds include those where:

at least one of R⁷ and R⁸ is —OH;

In other embodiments, the invention provides hydroxy compounds of formula I wherein X—Y is C═N. In some preferred embodiments, at least one of R¹ through R⁶ is —OH. In other preferred embodiments, at least one of R² through R⁴ is —OH.

In other embodiments, the invention provides glucuronyl compounds according to formula I, wherein at least one of R¹ through R⁶, R⁹, R¹⁰, and Z is —C(O)—O-G, —O-G, or -G.

In some preferred embodiments, the invention provides glucuronyl compounds where X—Y is CR⁹HNR¹⁰. In some preferred embodiments, R⁹ and R¹⁰ are H. In other preferred embodiments, at least one of Z, R³, and R⁴ is —O-G. In other preferred embodiments, at least one of R¹ through R⁶, R⁹, and Z is —O-G.

In still further embodiments, the invention provides glucuronyl compounds of formula I where R² along with R³ taken together form a double bond between the carbons to which they are attached, and at least one of R^{3′} and R⁴ is —O-G.

In other embodiments, the invention provides glucuronyl compounds of formula I where R¹⁰ is —C(O)O-G or -G. In some embodiments, such compounds are further provided where R⁴ and R^{4′} together with the carbon to which they are attached form C═O.

In some embodiments, the invention provides glucuronyl compounds where X—Y is —CHR⁹NR¹⁰ where R¹⁰ is —C(O)—O-G.

In some preferred embodiments, the invention provides compounds of formula I wherein Z, each Rⁿ and R^{n′} is H, X—Y is —CHR⁹NR¹⁰. In some such embodiments, R⁹ is H. In preferred embodiments, R⁹ is H and R¹⁰ is —C(O)—O-G.

In other embodiments, the invention provides glucuronyl compounds of formula I, where R¹⁰ is acetyl. In a preferred embodiment, such derivatives are further provided where at least one of R¹ through R⁶, R⁹, and Z is —O-G. In still other embodiments, at least one of R⁷ and R⁸ is —O-G.

In some embodiments, the invention provides sulfate compounds according to formula I where at least one of R¹ through R⁹, and Z is —OSO₃H.

In some preferred embodiments, the invention provides such sulfate compounds where X—Y is —CHR⁹NR¹⁰. In some such embodiments, R⁹ and R¹⁰ each are H. In some such embodiments, at least one of R¹ through R⁶ is —OSO₃H. In further embodiments, at least one of R² and R³ is —OSO₃H. In some embodiments, R³ is —OSO₃H.

In some embodiments, the invention provides sulfate compounds of formula I, where at least one of R⁹ and Z is —OSO₃H.

In some embodiments, the invention provides sulfamate compounds according to formula I. In some embodiments, the invention provides such sulfamate compounds where X—Y is CR⁹HNR¹⁰, and R¹⁰ is —SO₃H. In other embodiments, the invention provides such sulfamate compounds where at least two of Rⁿ and its corresponding Rⁿ⁺¹, where n=1-5, form a double bond between the carbons to which they are attached.

In some embodiments, the invention provides keto compounds according to formula I, where Rⁿ and its corresponding R^{n′} taken together with the carbon to which they are attached form C═O. In some preferred embodiments, n=4. In further preferred embodiments, X—Y is CR⁹HNR¹⁰ and preferably R¹⁰ is -G. In other embodiments, R⁹ and R¹⁰ are H.

Other embodiments of keto compounds according to formula I provide compounds where X—Y is C(O)NH.

In some embodiments, the invention provides imine compounds according to formula I. In some embodiments, X—Y is CH═N. In some such embodiments, at least one of R¹ through R⁶ is —OH. In other such embodiments, at least one of R² through R⁴ is —OH. In still other such embodiments, the compound may be an N-oxide, wherein the nitrogen between the carbons to which R⁶ and R⁷ are attached forms an N-oxide.

In still further embodiments, the invention provides dehydro compounds of formula I, containing one or more double bonds. In these embodiments, such compounds are provided wherein at least one of Rⁿ and its corresponding Rⁿ⁺¹, where n=1-5, together form a double bond between the carbons to which they are attached and each R^{n′} and R^{(n+1)′} is independently hydrogen, hydroxy, CH₃C(O)—O, —OSO₃H, or —O-G. In some preferred embodiments, n=2. In further preferred embodiments, n=2, R^{2′}═H, and R^{3′} or R⁴ is —O-G. In still further embodiments, X—Y is CHR⁹NR¹⁰, where R⁹ and R¹⁰ are preferably H.

In other embodiments, di-dehydro compounds of formula I are provided, where for at least two Rⁿ, each said Rⁿ and its corresponding Rⁿ⁺¹, where n=1-5, together form a double bond between the carbons to which they are attached. In some preferred embodiments, the invention further provides that X—Y═CHR⁹NR¹⁰. In further embodiments, R¹⁰ is H; and Z or R⁹ is —OSO₃H. In still other embodiments, X—Y═CHR⁹NR¹⁰ and R⁹═R¹⁰50 H.

In some embodiments, the invention provides such di-dehydro compounds of formula I where R¹⁰ is —SO₃H, or acetyl.

In some aspects of the invention, the compounds of formula I are provided in isolated form.

In other aspects of the inventions, the compounds of formula I are provided is substantially pure form of at least 75% purity. In other aspects, the compounds are at least 80% pure. In other aspects, the compounds are at least 85% pure. In other aspects, the compounds are at least 90% pure. In other aspects, the compounds are at least 95% pure.

Acetyl, as used herein, refers to CH₃—C(═O)—.

Alkyl, as used herein, refers to an aliphatic hydrocarbon chain, e.g., of 1 to 6 carbon atoms, and includes, but is not limited to, straight and branched chains such as methyl, ethyl, n-propyl, isopropyl, n-butyl, isobutyl, sec-butyl, t-butyl, n-pentyl, isopentyl, neo-pentyl, n-hexyl, and isohexyl. Lower alkyl refers to alkyl having 1 to 3 carbon atoms.

BOP refers to (Benzotriazol-1-yloxy)tris(dimethylamino)phosphonium hexafluorophosphate.

Carbamoyl, as used herein, refers to the group, —C(═O)N<.

DCC refers to N,N′-Dicyclohexylcarbodiimide.

DIBAH and DIBAL refer, interchangeably, to diisobutylaluminum hydride.

DMAP refers to 4-dimethylaminopyridine.

DPPA refers to diphenylphosphoryl azide.

EDC refers to 1-(3-dimethylaminopropyl)-3-ethylcarbodiimide hydrochloride.

Glucuronyl, as used herein, refers to the group:

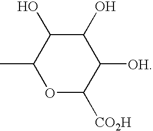

Halogen (or halo) as used herein refers to chlorine, bromine, fluorine and iodine. Hünig's Base is N,N-diisopropylethylamine, also indicated herein as i-Pr₂NEt.

PyBOP refers to (Benzotriazol-1-yloxy)tripyrrolidinophosphonium hexafluorophosphate.

The compounds of this invention contain asymmetric carbon atoms and thus give rise to optical isomers and diastereoisomers. The present invention includes such optical isomers and diastereoisomers; as well as the racemic and resolved, enantiomerically pure R and S stereoisomers; as well as other mixtures of the R and S stereoisomers and pharmaceutically acceptable salts thereof.

Where an enantiomer is preferred, it may, in some embodiments be provided substantially free of the corresponding enantiomer. Thus, an enantiomer substantially free of the corresponding enantiomer refers to a compound which is isolated or separated via separation techniques or prepared free of the corresponding enantiomer. “Substantially free,” as used herein, means that the compound is made up of a significantly greater proportion of one enantiomer. In preferred embodiments, the compound is made up of at least about 90% by weight of a preferred enantiomer. In other embodiments of the invention, the compound is made up of at least about 99% by weight of a preferred enantiomer. Preferred enantiomers may be isolated from racemic mixtures by any method known to those skilled in the art, including high performance liquid chromatography (HPLC) and the formation and crystallization of chiral salts or prepared by methods described herein. See, for example, Jacques, et al., Enantiomers, Racemates and Resolutions (Wiley Interscience, New York, 1981); Wilen, S. H., et al., Tetrahedron 33:2725 (1977); Eliel, E.L. Stereochemistry of Carbon Compounds (McGraw-Hill, N.Y., 1962); Wilen, S. H. Tables of Resolving Agents and Optical Resolutions p. 268 (E.L. Eliel, Ed., Univ. of Notre Dame Press, Notre Dame, Ind. 1972).

One skilled in the art will also recognize that it is possible for tautomers to exist of formula (I). The present invention includes all such tautomers even though not shown in formula (I).

The compounds useful in the present invention also include pharmaceutically acceptable salts of the compounds of formula (I). By “pharmaceutically acceptable salt”, it is meant any compound formed by the addition of a pharmaceutically acceptable base and a compound of formula (I) to form the corresponding salt. By the term “pharmaceutically acceptable” it is meant a substance that is acceptable for use in pharmaceutical applications from a toxicological perspective and does not adversely interact with the active ingredient. Pharmaceutically acceptable salts, including mono- and bi-salts, include, but are not limited to, those derived from such organic and inorganic acids such as, but not limited to, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, and similarly known acceptable acids.

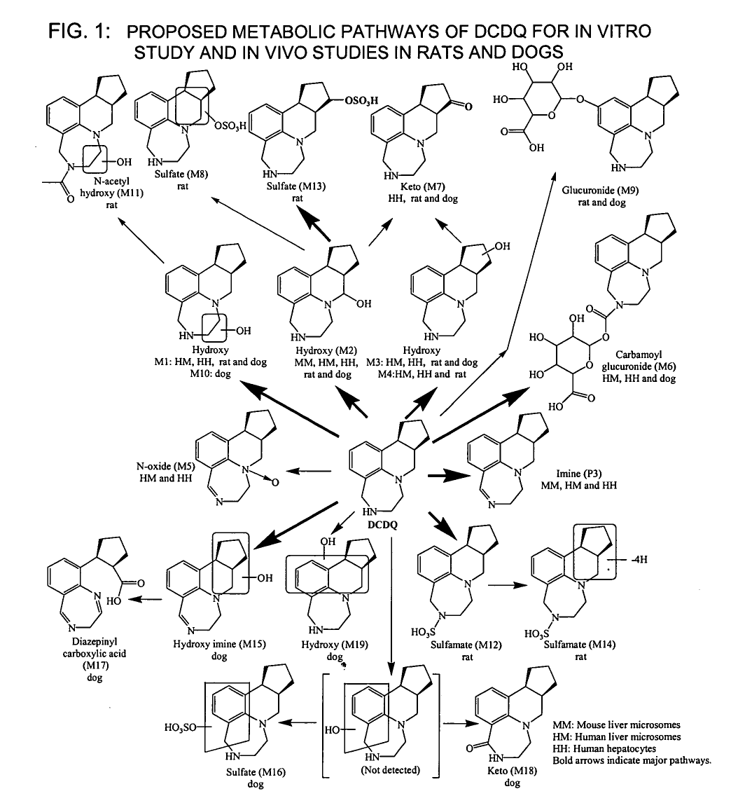

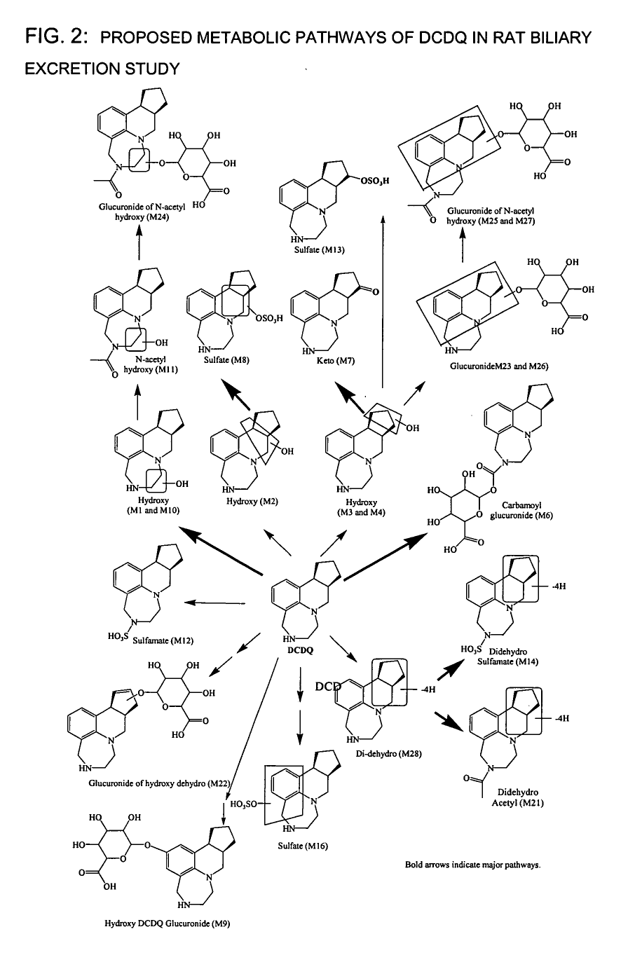

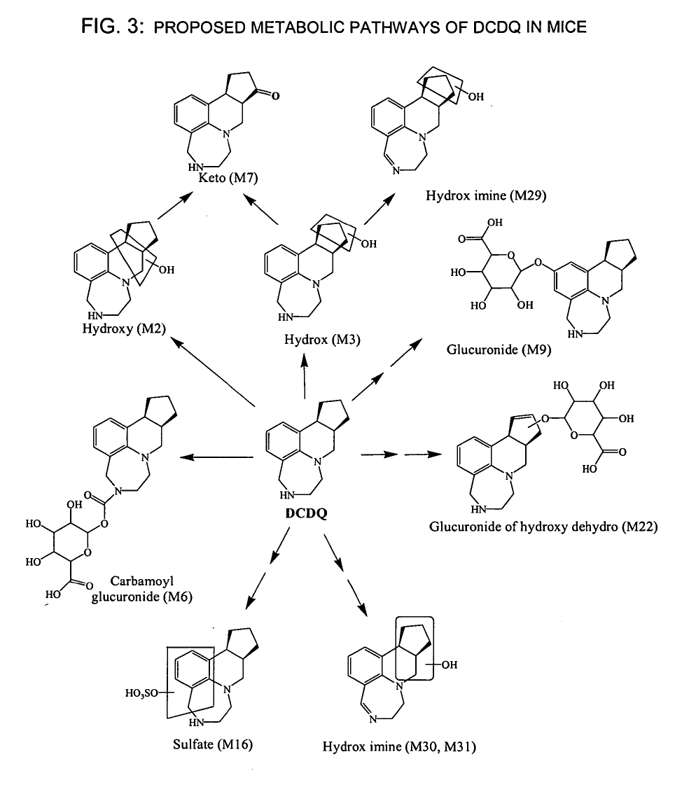

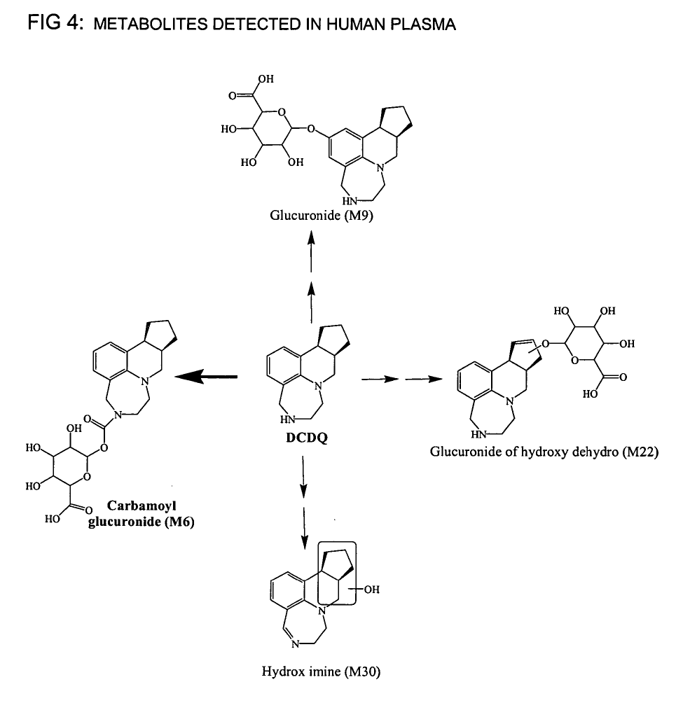

Non-limiting, examples of compounds of Formula I include those identified through the in vitro and in vivo studies detailed herein, and shown in the metabolic pathways depicted in FIGS. 1-4. Such examples include those shown below. Where the attachment of a given substituents is described by a box, it is intended that the indicated substituent can be attached to any one or more available carbon atoms within the box.
Hydroxy Metabolites 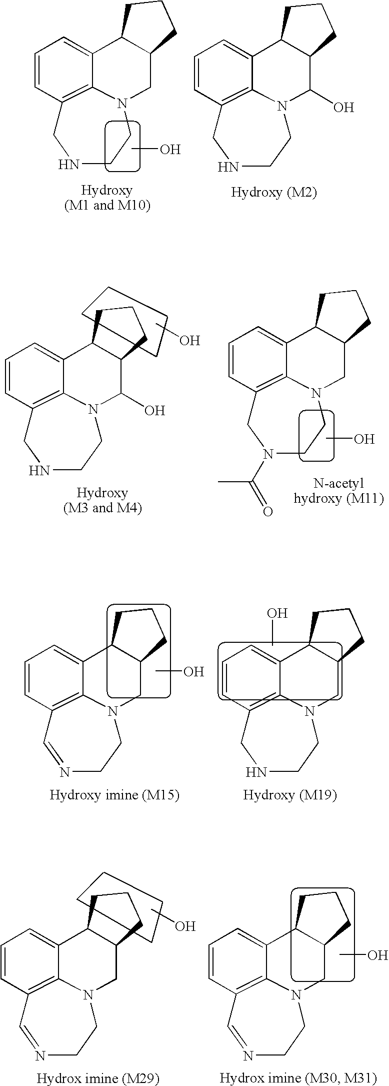
Glucuronyl Metabolites 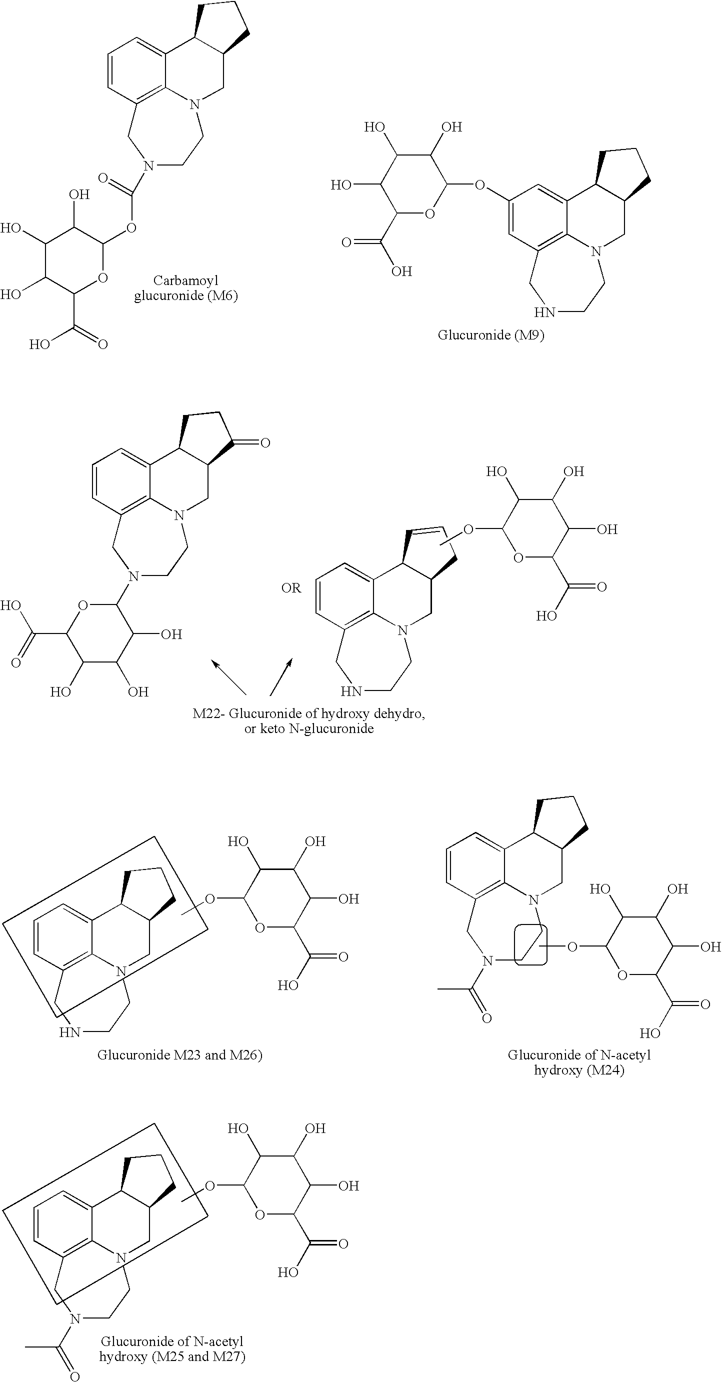
Sulfate Compounds 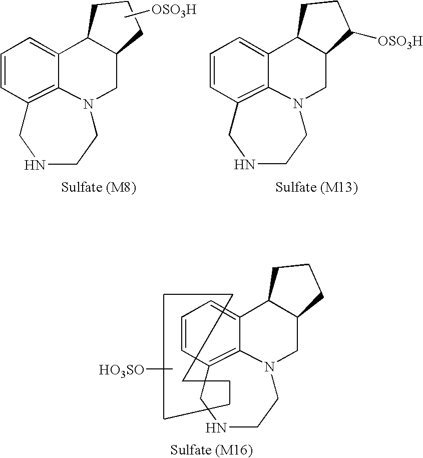
Sulfamate Compounds 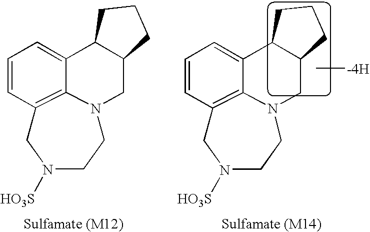
Keto Compounds 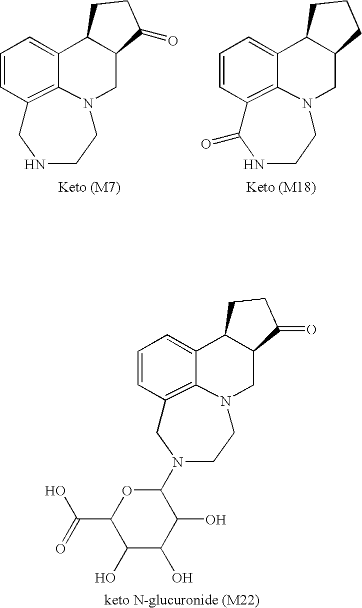
Imine Compounds 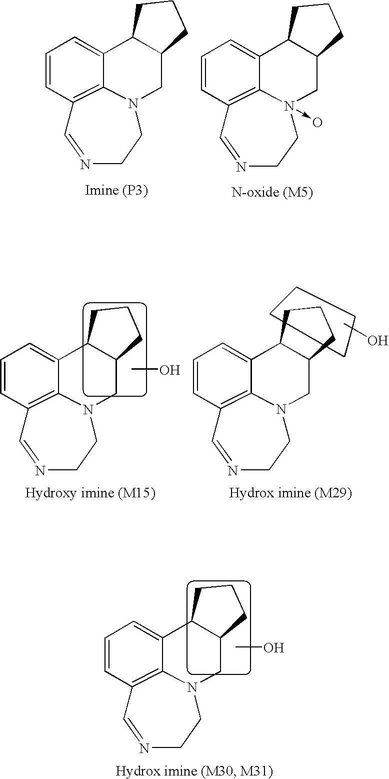
Dehydro Compounds

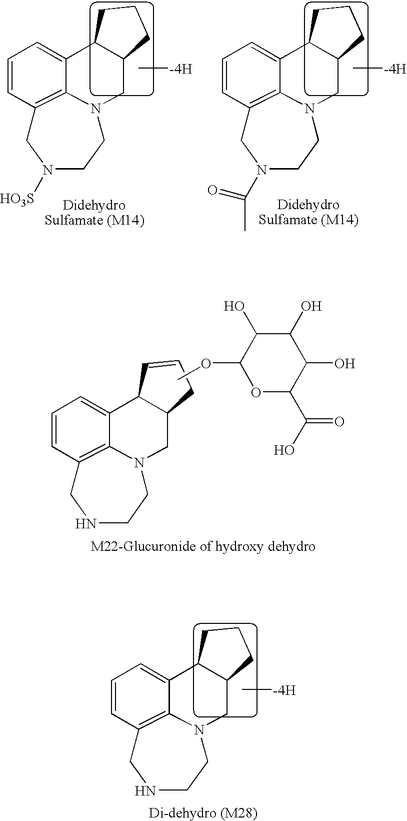

### Exemplary Syntheses

Proposed Synthesis for Hydroxy Metabolite M1 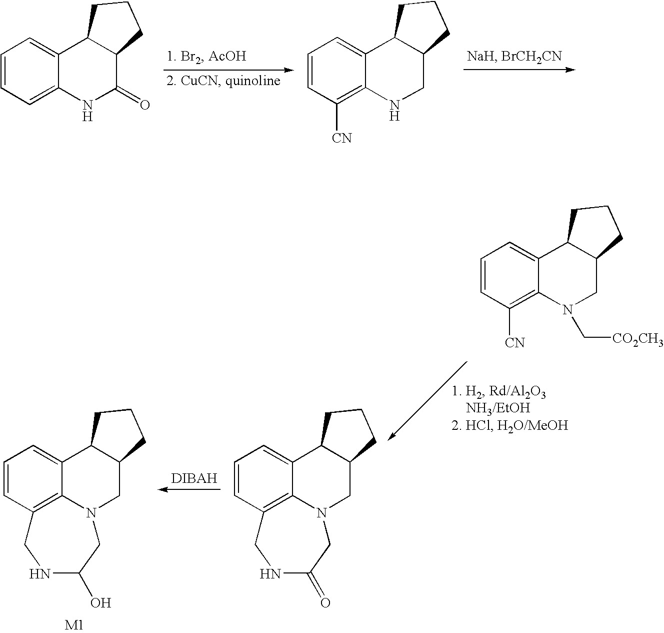
Proposed Synthesis of Hydroxy Metabolite M2 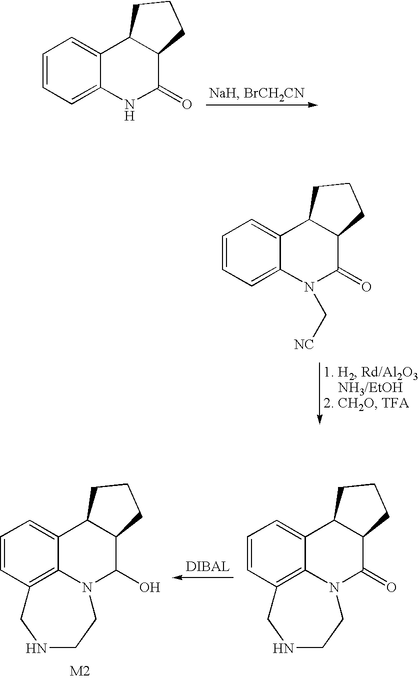
Proposed Synthesis of N-Oxide Metabolite M5 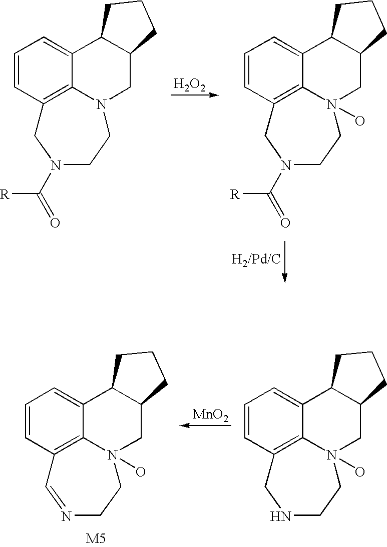
Synthesis for Carabamoyl Glucuronyl Metabolite M6 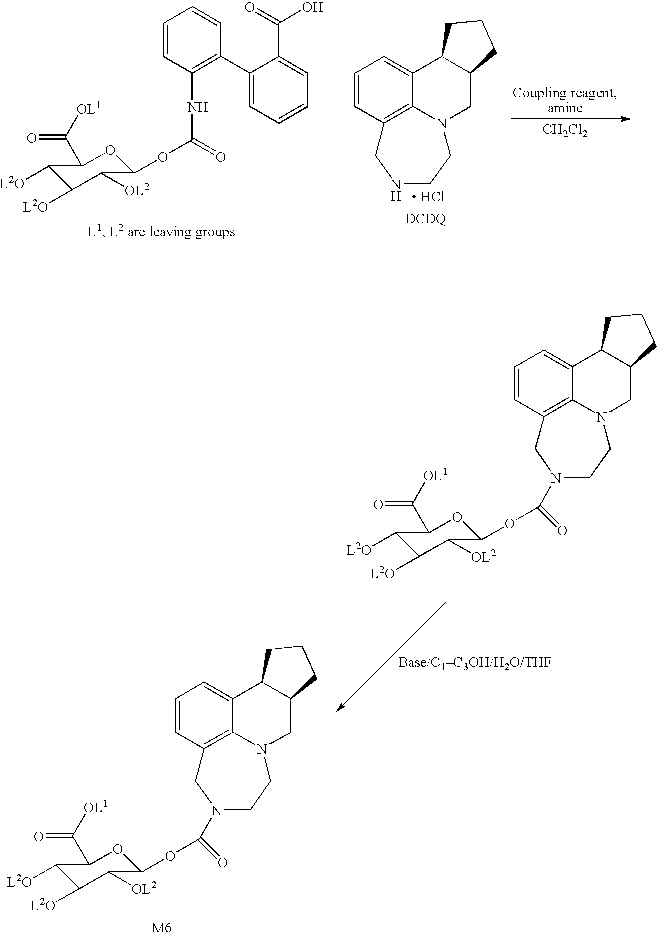
Proposed Syntheses for Hydroxy Metabolites M3 And M4, Keto Metabolite M7, And Sulfate Metabolites M8 And M13 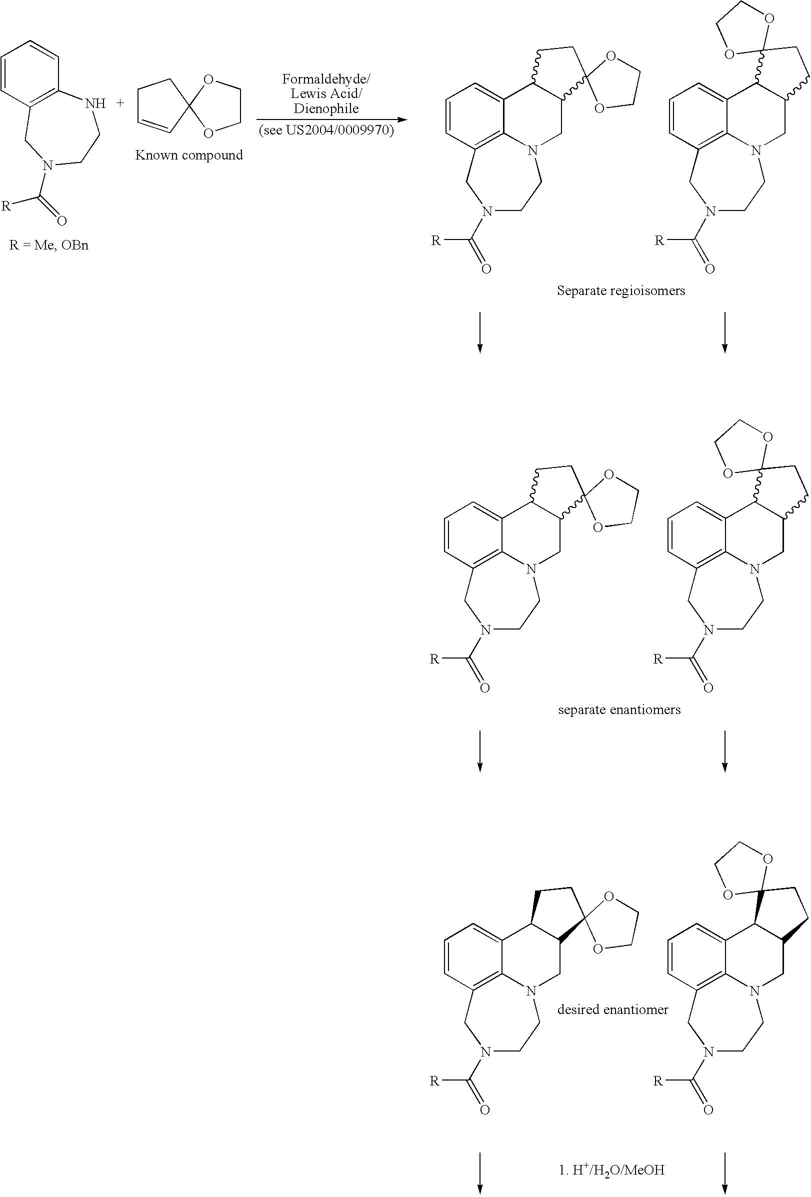 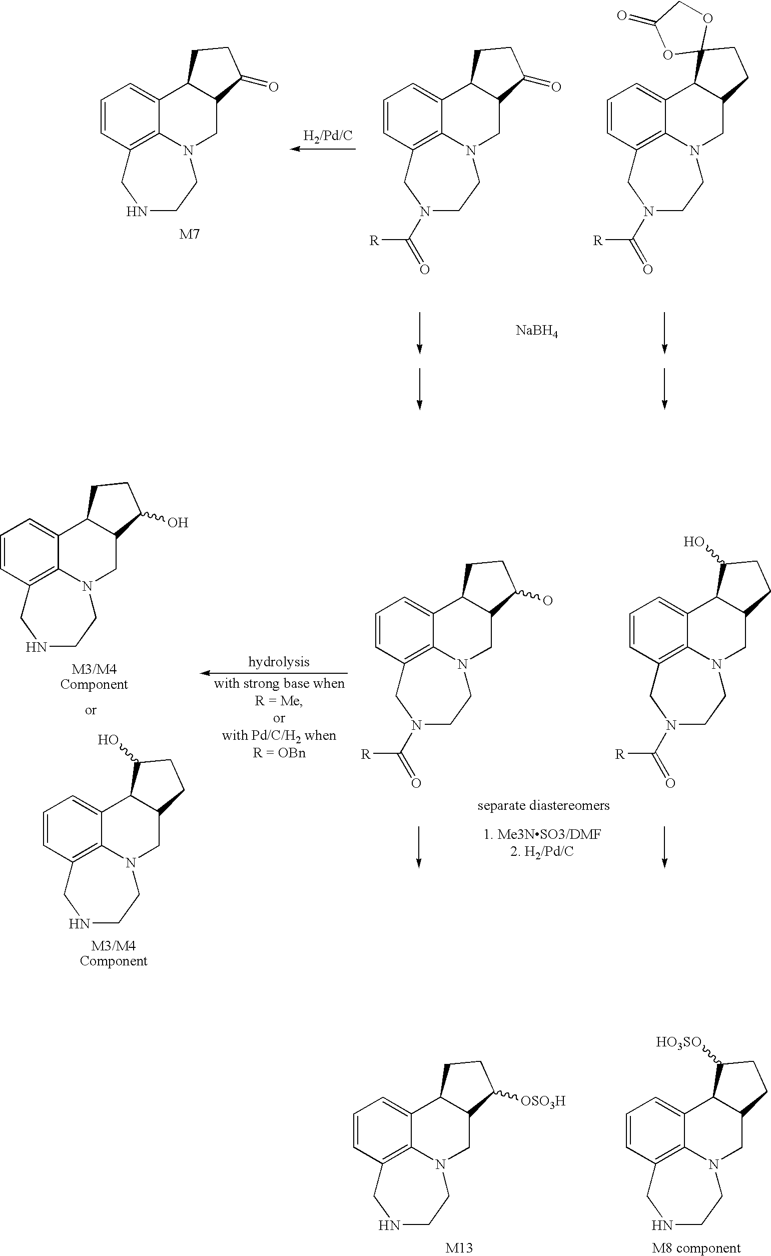
Proposed Synthesis of Additional Component of Sulfate Metabolite M8 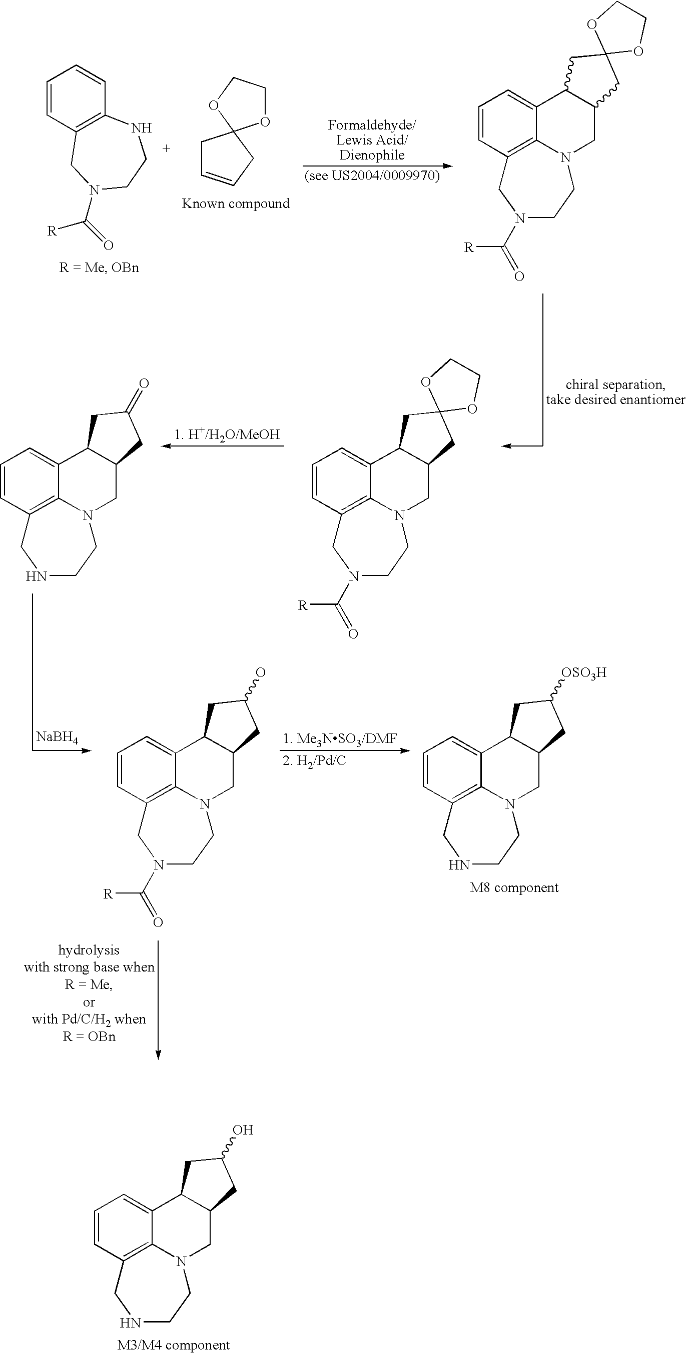
Proposed Synthesis of Glucuronyl Metabolite M9 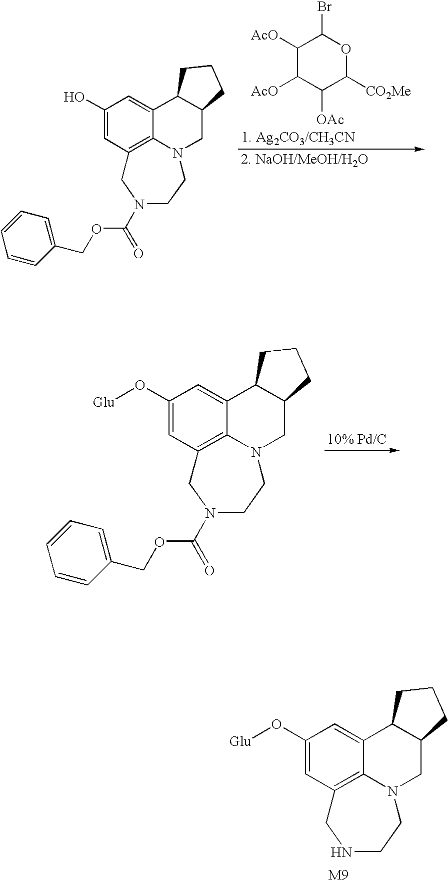
Proposed Synthesis of Hydroxy Metabolite M10 and N-Acetyl Hydroxy Metabolite M11 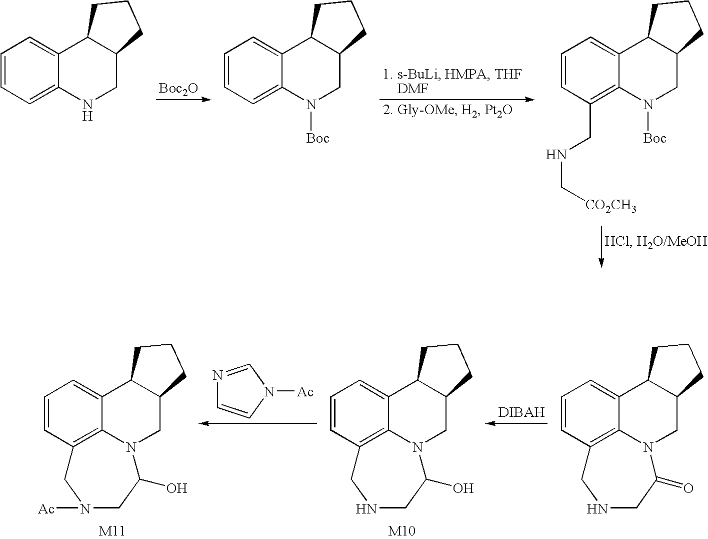
Proposed Synthesis of Sulfamate Metabolite M12 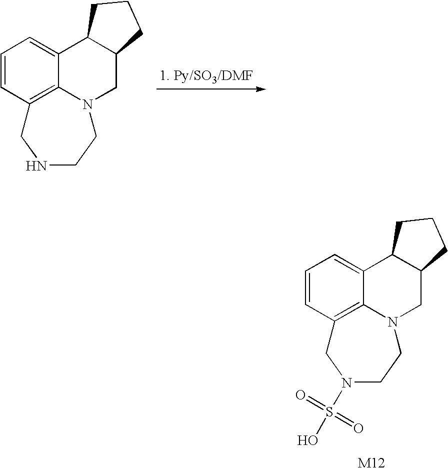
Proposed Syntheses for Sulfamate Metabolite M12, Di-Dehydro Sulfamate M14, and N-Acetyl Di-Dehydro Metabolite M21 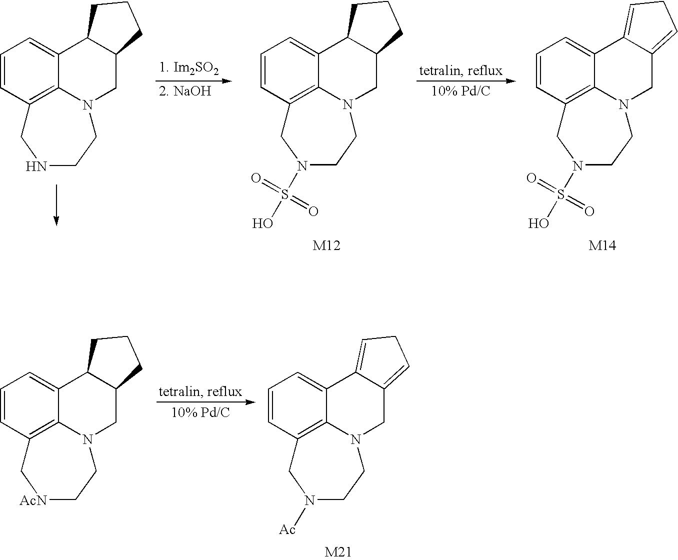
Proposed Synthesis of Keto Metabolite M18 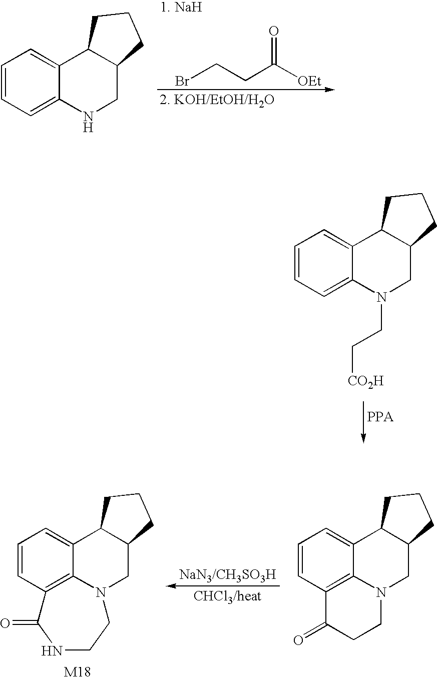
Proposed Syntheses for Hydroxy Imine Metabolites M15, and M29-M31 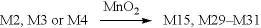
Proposed Synthesis for Sulfamate Metabolite M16 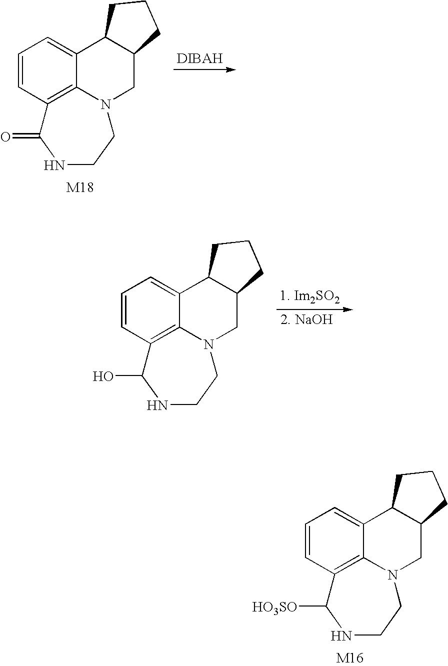
Proposed Synthesis of Imine Metabolite P3

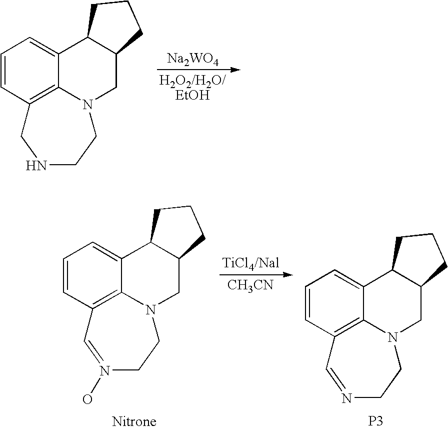

These proposed syntheses are exemplary only. Those of skill in the art will recognize that other syntheses may be used to make the various compounds of the invention. Additionally, those of skill in the art will recognize that an intermediate in any the schemes described above may be a compound according to Formula I and may be collected and purified, if necessary, without going to the next step. For example, the nitrone above may be isolated and purified. Furthermore, those of skill in the art will recognize that these syntheses may be modified to yield related compounds which are described by Formula I, herein. These and other variations or modifications of these methods, compounds, and intermediates are considered with the scope and spirit of the invention disclosed herein.

Methods of Treatment

The binding affinity of DCDQ, and related compounds, is well-documented in the related published applications WO03/091250 and US2004/0009970, each of which is incorporated by reference. Accordingly, the metabolites, which form after administration of DCDQ, can also be used similarly to DCDQ in treating psychotic and other disorders.

The compounds of this invention are agonists and partial agonists at the 2C subtype of brain serotonin receptors and are thus of interest for the treatment of mental disorders, including psychotic disorders such as schizophrenia including paranoid type, disorganized type, catatonic type, and undifferentiated type, schizophreniform disorder, schizoaffective disorder, delusional disorder, substance-induced psychotic disorder, and psychotic disorder not otherwise specified; L-DOPA-induced psychosis; psychosis associated with Alzheimer's dementia; psychosis associated with Parkinson's disease; psychosis associated with Lewy body disease; bipolar disorders such as bipolar I disorder, bipolar II disorder, and cyclothymic disorder; depressive disorders such as major depressive disorder, dysthymic disorder, substance-induced mood disorder, and depressive disorder not otherwise specified; mood episodes such as major depressive episode, manic episode, mixed episode, and hypomanic episode; anxiety disorders such as panic attack, agoraphobia, panic disorder, specific phobia, social phobia, obsessive compulsive disorder, posttraumatic stress disorder, acute stress disorder, generalized anxiety disorder, separation anxiety disorder, substance-induced anxiety disorder, and anxiety disorder not otherwise specified; adjustment disorders such as adjustment disorders with anxiety and/or depressed mood; intellectual deficit disorders such as dementia, Alzheimer's disease, and memory deficit; eating disorders (e.g., hyperphagia, bulimia or anorexia nervosa) and combinations of these mental disorders that may be present in a mammal. For example, mood disorders such as depressive disorders or bipolar disorders often accompany psychotic disorders such as schizophrenia. A more complete description of the aforementioned mental disorders can be found in the Diagnostic and Statistical Manual of Mental Disorders, 4th edition, Washington, D.C., American Psychiatric Association (1994).

The compounds of the present invention are also of interest for the treatment of epilepsy; migraines; sexual dysfunction; sleep disorders; gastrointestinal disorders, such as malfunction of gastrointestinal motility; and obesity, with its consequent comorbidities including Type II diabetes, cardiovascular disease, hypertension, hyperlipidemia, stroke, osteoarthritis, sleep apnea, gall bladder disease, gout, some cancers, some infertility, and early mortality. The compounds of the present invention can also be used to treat central nervous system deficiencies associated, for example, with trauma, stroke, and spinal cord injuries. The compounds of the present invention can therefore be used to improve or inhibit further degradation of central nervous system activity during or following the malady or trauma in question. Included in these improvements are maintenance or improvement in motor and motility skills, control, coordination and strength.

Thus the present invention provides methods of treating each of the maladies listed above in a mammal, preferably in a human, the methods comprising providing a therapeutically effective amount of a compound of this invention to the mammal in need thereof. By “treating”, as used herein, it is meant partially or completely alleviating, inhibiting, preventing, ameliorating and/or relieving the disorder. For example, “treating” as used herein includes partially or completely alleviating, inhibiting or relieving the condition in question. “Mammals” as used herein refers to warm blooded vertebrate animals, such as humans. “Provide”, as used herein, means either directly administering a compound or composition of the present invention, or administering a derivative or analog which will form an equivalent amount of the active compound or substance within the body.

Pharmaceutical Compositions

Also encompassed by the present invention are pharmaceutical compositions for treating or controlling disease states or conditions of the central nervous system comprising at least one compound of Formula I, mixtures thereof, and or pharmaceutical salts thereof, and a pharmaceutically acceptable carrier therefore. Such compositions are prepared in accordance with acceptable pharmaceutical procedures, such as described in Remingtons Pharmaceutical Sciences, 17th edition, ed. Alfonoso R. Gennaro, Mack Publishing Company, Easton, Pa. (1985). Pharmaceutically acceptable carriers are those that are compatible with the other ingredients in the formulation and biologically acceptable.

The compounds of this invention may be administered orally or parenterally, neat or in combination with conventional pharmaceutical carriers, the proportion of which is determined by the solubility and chemical nature of the compound, chosen route of administration and standard pharmacological practice. The pharmaceutical carrier may be solid or liquid.

Applicable solid carriers can include one or more substances which may also act as flavoring agents, lubricants, solubilizers, suspending agents, fillers, glidants, compression aids, binders or tablet-disintegrating agents or an encapsulating material. In powders, the carrier is a finely divided solid which is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Liquid carriers may be used in preparing solutions, suspensions, emulsions, syrups and elixirs. The active ingredient of this invention can be dissolved or suspended in a pharmaceutically acceptable liquid carrier such as water, an organic solvent, a mixture of both or pharmaceutically acceptable oils or fat. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols e.g. glycols) and their derivatives, and oils (e.g. fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

Liquid pharmaceutical compositions which are sterile solutions or suspensions can be administered by, for example, intramuscular, intraperitoneal or subcutaneous injection. Sterile solutions can also be administered intravenously. Oral administration may be either liquid or solid composition form.

The compounds of this invention may be administered rectally or vaginally in the form of a conventional suppository. For administration by intranasal or intrabronchial inhalation or insufflation, the compounds of this invention may be formulated into an aqueous or partially aqueous solution, which can then be utilized in the form of an aerosol. The compounds of this invention may also be administered transdermally through the use of a transdermal patch containing the active compound and a carrier that is inert to the active compound, is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semipermeable membrane covering a reservoir containing the active ingredient with or without a carrier, or a matrix containing the active ingredient. Other occlusive devices are known in the literature.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example packeted powders, vials, ampoules, prefilled syringes or sachets containing liquids. The unit dosage form can be, for example, a capsule or tablet itself, or it can be the appropriate number of any such compositions in package form.

The dosage requirements vary with the particular compositions employed, the route of administration, the severity of the symptoms presented and the particular subject being treated. Based on the results obtained in the standard pharmacological test procedures, projected estimated daily dosages of active compound would be approximately 0.02 μg/kg—approximately 4000 μg/kg, or up to approximately 500 mg/day. It is to be understood that these dosage ranges are merely estimates and those of skill in the art will be able to ascertain appropriate doses depending on many factors, including patient weight, severity of symptoms, and other factors. Treatment will generally be initiated with small dosages less than the optimum dose of the compound. Thereafter the dosage is increased until the optimum effect under the circumstances is reached; precise dosages for oral, parenteral, nasal, or intrabronchial administration will be determined by the administering physician based on experience with the individual subject treated.

### EXAMPLES

Metabolite Compounds

The metabolism of DCDQ was investigated in several in vitro and in vivo models by using a radio-labeled version of DCDQ, [¹⁴C]DCDQ. The studies revealed several metabolic pathways and several significant metabolites. These studies are explained in further detail in the Experimental section, below.

The metabolism of [¹⁴C]DCDQ was investigated by incubation with liver microsomes from male and female CD-1 mice, Sprague Dawley rats, beagle dogs and human liver microsomes pooled across sexes, and cryopreserved male human hepatocytes. DCDQ was converted to oxidative metabolites, including M1, M2, M3, M4, M5, and a carbamoyl glucuronide (M6) in microsomal incubations and human hepatocytes.

The in vivo metabolism of [¹⁴C]DCDQ was further investigated in four male beagle dogs following a single administration of 14.1 to 16.7 mg/kg of [¹⁴C]DCDQ hydrochloride in an enteric coated capsule. The major metabolites observed in plasma included hydroxy DCDQ (M1, M2 and M3), an N-oxide DCDQ (M5), a keto DCDQ (M7), a hydroxy DCDQ imine (M15), a hydroxy DCDQ glucuronide (M9) and the carbamoyl glucuronide of DCDQ (M6). A sulfate conjugate of hydroxy DCDQ (M16) and a diazepinyl DCDQ carboxylic acid (M17), which were not detected in plasma, were observed in urine samples. Hydroxy DCDQ metabolites (M2, M3 and M19), a keto DCDQ (M18) and the hydroxy DCDQ imine (M15) were detected in fecal extracts. DCDQ was extensively metabolized in dogs, with the oxidative metabolism as the major metabolic pathway, although formation of a DCDQ carbamoyl glucuronide (M6) was also observed.

The in vivo metabolism of [¹⁴C]DCDQ was further studied in male and female Sprague-Dawley rats after a single oral administration (5 mg/kg). Metabolites detected in plasma included hydroxy DCDQ metabolites (M1, M2, M3, M4 and M10), keto DCDQ (M7), and the phase 11 metabolites DCDQ sulfamate (M12), di-dehydro DCDQ sulfamate (M14), hydroxy DCDQ sulfates (M8 and M13), hydroxy DCDQ glucuronide (M9) and acetylated hydroxy DCDQ (M11). DCDQ was extensively metabolized in rats to predominantly oxidative metabolites.

Thus, metabolites of DCDQ are created through several metabolic pathways, some of which are common across several species. Such metabolites can be useful in treating disorders and diseases affected by the 5HT_{2C} receptor and/or those that can be treated by administration of DCDQ.
Synthesis of the Carbamoyl Glucuronyl Metabolite (M6)

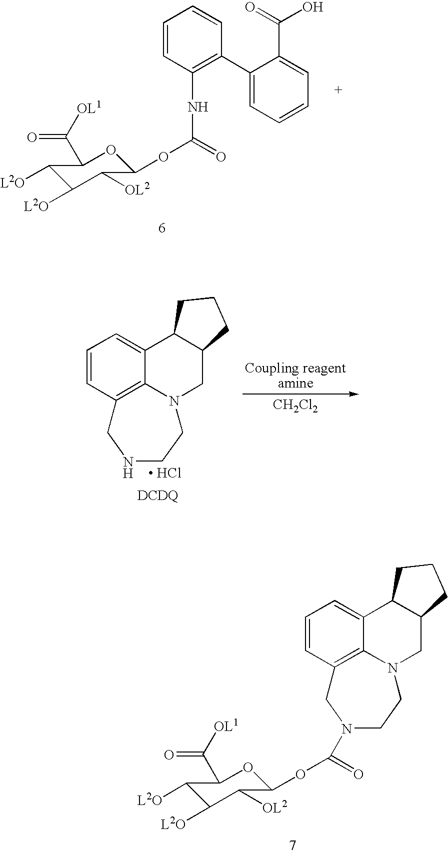

Metabolite M6 can be obtained by coupling DCDQ with a glucuronyl carrier 6 in the presence of a coupling reagent and an amine in CH₂Cl₂ to yield compound 7. The product, compound 7 can be purified according to methods known in the art, and preferably by column chromatography purification, preferably with EtOAc/heptane as an eluent. The coupling reagent can be selected from any suitable coupling reagent, including but not limited to BOP, DCC, and EDC. BOP is the preferred coupling agent. Suitable amines include, but are not limited to Et₃N, pyridine, and Hünig's base. Hunig's base is preferred. The glucuronyl carrier 6 can be prepared by methods known to those of skill in the art. L¹ is an aliphatic leaving group, such as, but not limited to, C₁ to C₆ alkyl, methyl, ethyl, and propyl, preferably methyl. Each L² is a leaving group which is independently selected from an acetyl group and a benzyllic group. Acetyl groups are preferred. The glucuronyl carrier 6 is preferably a secondary amine glucuronyl carbamate 6 such as those that can be designed on the basis of the Scheeren's protocol discussed in Ruben G. G. Leeders, Hans W. Scheeren, Tetrahedron Letters 2000, 41, 9173-9175.
Glucuronyl Carbamoyl Metabolite M6

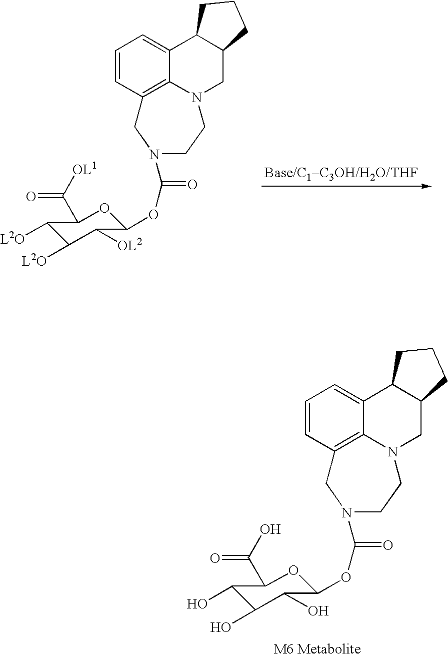

Compound 7 is then subjected to basic hydrolysis resulting in deprotection of all leaving groups, L², on 2,3,4-position of sugar moiety as well as L¹ to yield the final product M6 metabolite. Basic hydrolysis is carried out using base, such as NaOH, LiOH, and KOH in C₁-C₃ aliphatic alcohol. LiOH is the preferred base and MeOH is the preferred alcohol. Removal of organic solvents and lyophilization can be used to yield crude product M6 in a quantitative yield. Purification of the crude M6 can then be carried out by methods known to those of skill in the art.
Glucuronyl Carrier, Compound 6

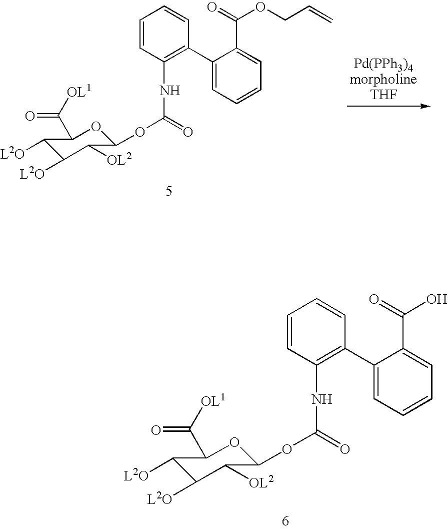

Compound 6 can be prepared by deprotection of the allyl group in compound 5 catalyzed preferably by using Pd(PPh₃)₄, and morpholine as a nucleophile. Fresh catalyst is preferred. Additionally N₂ may optionally be bubbled through the reaction solution before adding catalyst. In this way, the crude glucuronyl carrier 6 is obtained in a quantitative yield without further purification.
Compound 5

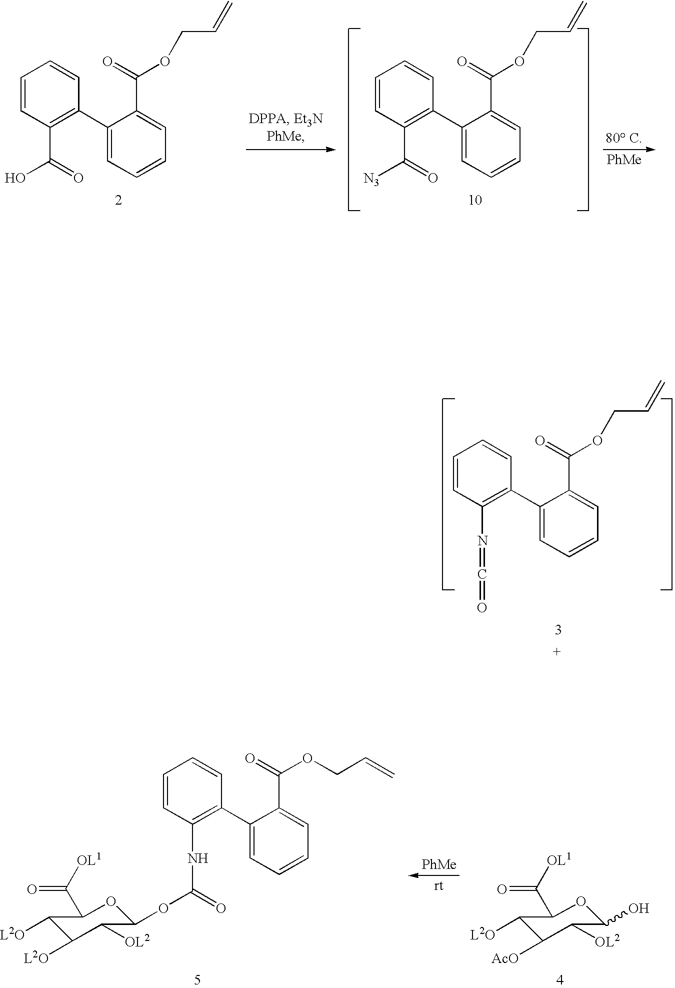

Compound 5 can be prepared in high yield in a one-pot reaction. Treatment of compound 2 with one of DPPA, NaN₃, or TMSN₃ in the presence of Et₃N in toluene in situ produces an acyl azide 10, which is heated, preferably to 80° C. for 1.5 hour, to yield isocyanate 3. The compound 3 need not be isolated and is subsequently treated with a 1-hydroxyglucuronic ester 4, preferably at room temperature overnight to obtain the title compound 5 (Scheme 3). Compound 4 can be prepared by following the procedure described U.S. Pat. No. 6,380,166B1, which is hereby incorporated by reference. ¹H NMR at 30° C. shows that all signals are double due to restricted rotation around the Ar—Ar bond.

In compound 4, L¹ is an aliphatic leaving group, such as, but not limited to, C₁ to C₆ alkyl, methyl, ethyl, and propyl, preferably methyl. Each L² is a leaving group which is independently selected from an acetyl group and a benzyllic group. Acetyl groups are preferred.

Compound 2

In order to prepare monoallyl ester 2, diphenic anhydride was chosen as a starting material and treated with excess of allyl alcohol in the presence of catalyst. Sutiable catalysts include Et₃N, Hünig's base, pyridine, amines, NaOH, LiOH, KPH, and other inorganic bases. Quantitative yields for compound 2 can be achieved.
Compound 5

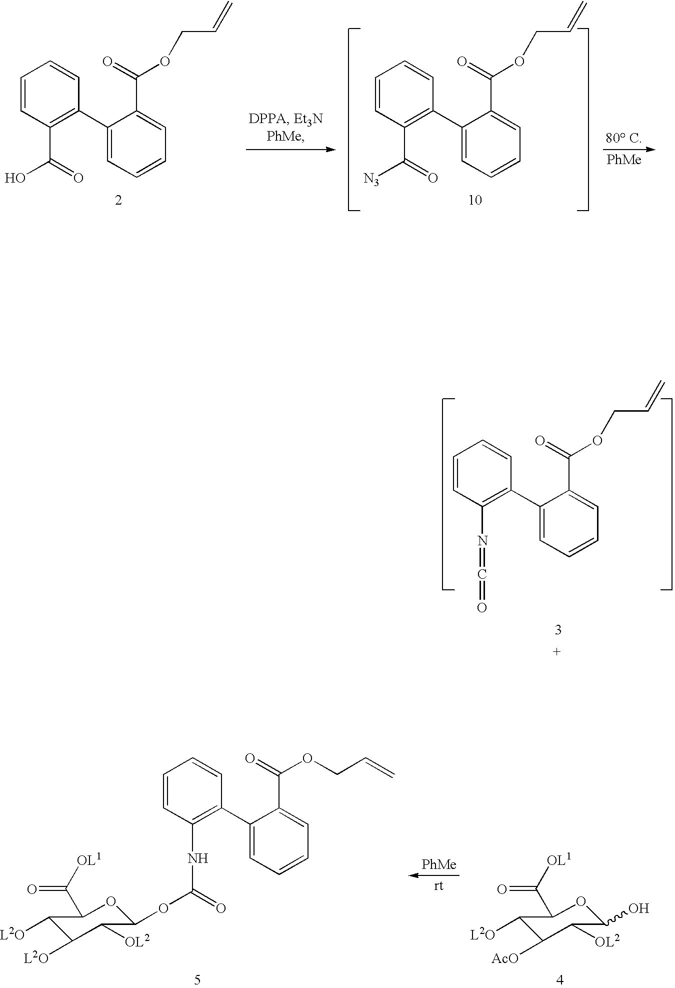

Compound 5 can be prepared in high yield in a one-pot reaction. Treatment of compound 2 with one of DPPA, NaN₃, or TMSN₃ in the presence of Et₃N in toluene in situ produces an acyl azide 10, which is heated, preferably to 80° C. for 1.5 hour, to yield isocyanate 3. The compound 3 need not be isolated and is subsequently treated with a 1-hydroxyglucuronic ester 4, preferably at room temperature overnight to obtain the title compound 5 (Scheme 3). Compound 4 can be prepared by following the procedure described U.S. Pat. No. 6,380,166B1, which is hereby incorporated by reference. ¹H NMR at 30° C. shows that all signals are double due to restricted rotation around the Ar—Ar bond.

In compound 4, L¹ is an aliphatic leaving group, such as, but not limited to, C₁ to C₆ alkyl, methyl, ethyl, and propyl, preferably methyl. Each L² is a leaving group which is independently selected from an acetyl group and a benzyllic group. Acetyl groups are preferred.

### Exemplary Synthesis of the Carbamoyl Glucoronyl Metabolite (M6)

An exemplary synthesis of DCDQ carbamoyl glucuronide metabolite (M6) is shown in Scheme 1: 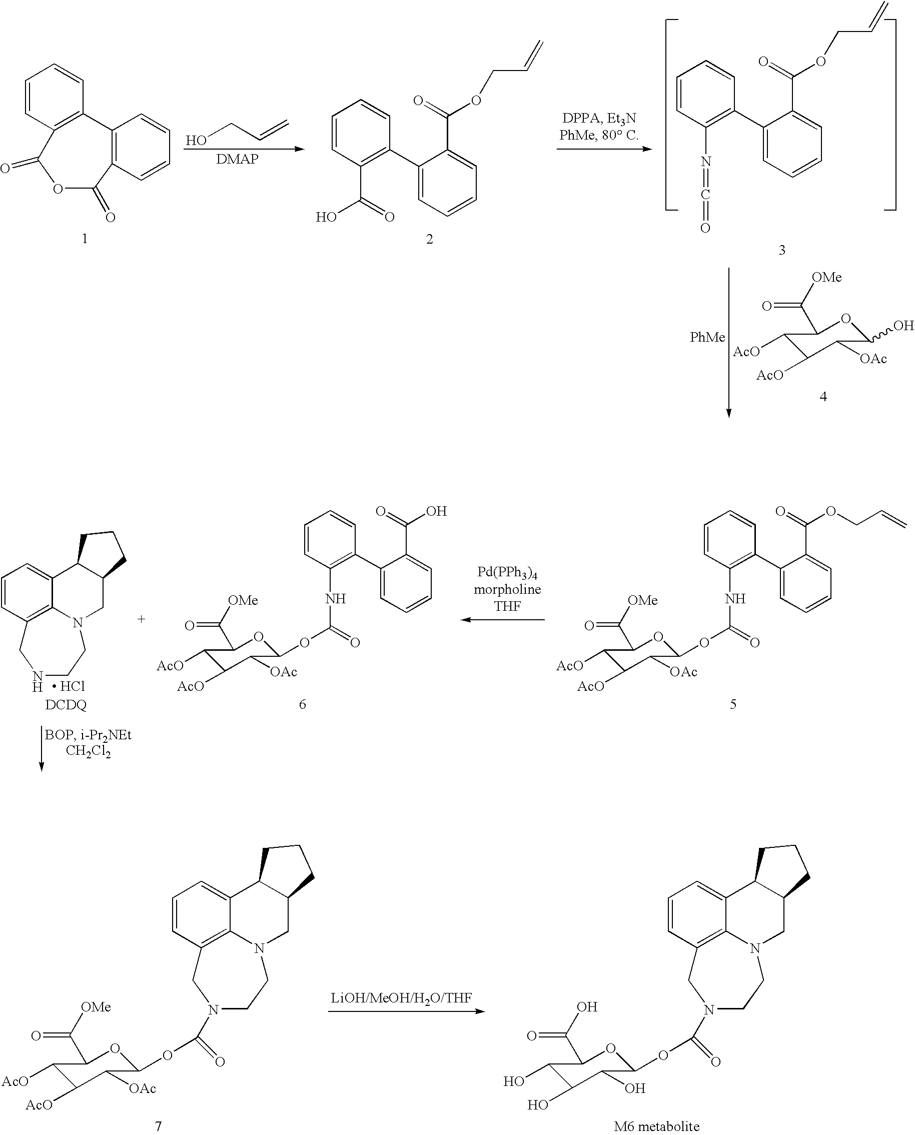
General

NMR spectra were recorded on a Varian Inova 300 at 300 MHz (¹H and ¹³C) and chemical shifts were identified in ppm relative to TMS internal standard. Analytical and preparative TLCs were performed on Silica Gel 60 F-254 pre-coated plates obtained from EM Science. Compounds were visualized using UV at 254 nm or 10% aq. KMnO₄ indicator. HPLC analysis was determined on a Waters Alliance 2695 HPLC instrument equipped with a PDA (Model 2996) UV detector. Mass spectra were recorded on a Finnigan mass spectrometer.

### BIPHENYL-2,2′-DICARBOXYLIC ACID 2′-ALLYL ESTER 2

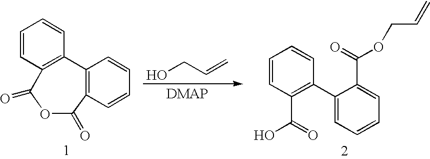

To a 1-L flask was charged diphenic anhydride (40 g, 178 mmol), allyl alcohol (300 mL) and DMAP (2.18 g, 17.8 mmol, 10 mol %). The reaction mixture was stirred for 12 h. The excess of allyl alcohol was evaporated under reduced pressure at 40° C. The residue was redissolved in EtOAc (400 mL) and washed with aq. NaHSO₄ (0.5 N, 200 mL), brine (200 mL×3) and water (200 mL×3). The organic layer was dried with anhydrous Na₂SO₄, passed through a silica gel pad (500 g), washed the pad with EtOAc (1 L), concentrated under reduced pressure to dryness. Traces of allyl alcohol were removed by distillation with heptane to give the mono allyl ester 2 (50 g, 100%) as a colorless oil. ¹H NMR (300 MHz, CDCl₃): 8.03-7.99 (m, 2H), 7.56-7.39 (m, 4H), 7.19-7.16 (m, 2H), 5.74-5.61 (m, 1H), 5.17-5.06 (m, 2H), 4.52-4.49 (m, 2H).

### 3,4,5-TRIACETOXY-6-(2′-ALLYLOXYCARBONYLBIPHENYL-2-YLCARBAMOYLOXY)TETRAHYDRO-PYRAN-2-CARBOXYLIC ACID METHYL ESTER 5

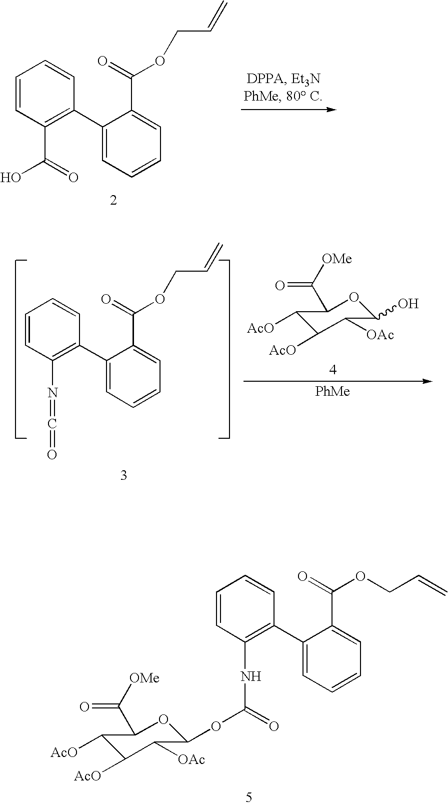

To a 500 mL-flask was charged biphenyl-2,2′-dicarboxylic acid 2′-allyl ester 2 (5.2 g, 18.4 mmol), toluene (100 mL), DPPA (4.8 mL, 22.1 mmol, 1.2 eq) and Et₃N (3.1 mL, 22.1 mmol, 1.2 eq) under nitrogen atmosphere. The reaction mixture was stirred overnight at room termperature, then heated to 85° C. for 1.5 h to generate in situ intermediate isocyanate 3. The mixture was cooled to room temperature. To this mixture was added methyl 2,3,4-triacetyl-1-hydroxyglucuronic ester 4 (3.7 g, 11 mmol, 0.6 eq) and stirred overnight. The mixture was diluted with EtOAc (500 mL), washed subsequently with aq. NaHSO₄ (0.5 N, 200 mL), saturated NaHCO₃ (200 mL), brine (200 mL×2) and water (200 mL). The organic layer was dried over anhydrous NaSO₄ and concentrated under reduced pressure. The residue (11 g), mixed with silica gel (22 g), was loaded on a column (4.5×50 cm) which was packed with silica gel (500 g). The column was washed with EtOAc/heptane (2:8, 6 L; 3:7, 4 L; 4:6, 4 L). Fractions (60 mL/fraction) were collected and solvent was evaporated to give compound 5 (5.5 g, 82%). HPLC, RT=7.73 min, purity: 81.44%. ¹HNMR (300 MHz, CDCl₃), all signals are double due to restricted rotation around the Ar—Ar bond, 8.03-7.93 (m, 2H), 7.64-7.48 (m, 2H), 7.40-7.23 (m, 2H), 7.18-7.05 (m, 2H), 6.49, 6.42 (2s, ¹H, NH), 5.74, 5.73 (2d, J=8.1 Hz, ¹H, β-anomer), 5.70-5.57 (m, ¹H), 5.33-5.01 (m, 5H), 4.54-4.48 (m, 2H), 4.16 (d, J=9.9 Hz, ¹H), 3.73, 3.72 (2s, 3H), 2.04-1.95 (3s, 9H). MS: m/z:[636 M+Na]⁺.

### 3,4,5-TRIACETOXY-6-(2′-CARBOXYLBIPHENYL-2-YLCARBAMOYLOXY)TETRAHYDROPYRAN-2-CARBOXYLIC ACID METHYL ESTER 6

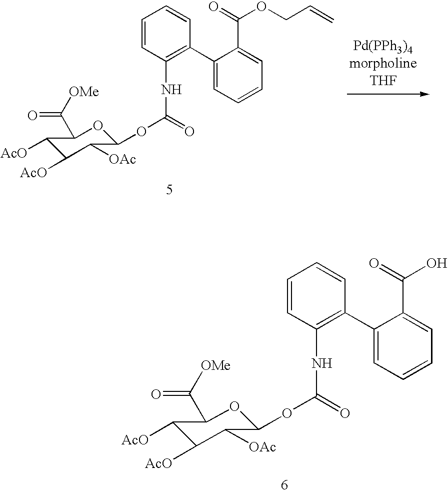

To a 500-mL flask was charged 3,4,5-Triacetoxy-6-(2′-allyloxycarbonylbiphenyl-2-ylcarbamoyloxy)tetrahydro-pyran-2-carboxylic acid methyl ester 5 (5.3 g, 8.65 mmol), THF (400 mL) and morpholine (3.8 mL, 43.3 mmol, 5 eq). The reaction mixture was stirred for 2 h at room temperature while bubbling nitrogen through the solution. After that, Pd(PPh₃)4 (300 mg, 0.26 mmol, 3 mol %) was added. The reaction mixture was further stirred for 15 min, diluted with Et₂O (1 L), and washed with NaHSO₄ (0.5 N, 300 mL), brine (300 mL×2), water (400 mL×2). The organic layer was dried over MgSO₄ and evaporated to obtain compound 6 (5.3 g, 100%, HPLC: 84% purity). This compound was used without further purification in the next step. ¹HNMR (300 MHz, CDCl₃): all signals are double due to restricted rotation around the Ar—Ar bond, 8.05-7.15 (m, 8H), 5.72, 5.70 (2d, J=8.1 Hz, ¹H), 5.36-5.02 (m, 2H), 4.18, 4.13 (2d, J=9.9 Hz, ¹H), 3.77-3.73 (m, ¹H), 3.72 (s, 3H), 2.03-1.98 (3s, 9H). MS: m/z: 572 [M−H]⁻.

### Compound 7

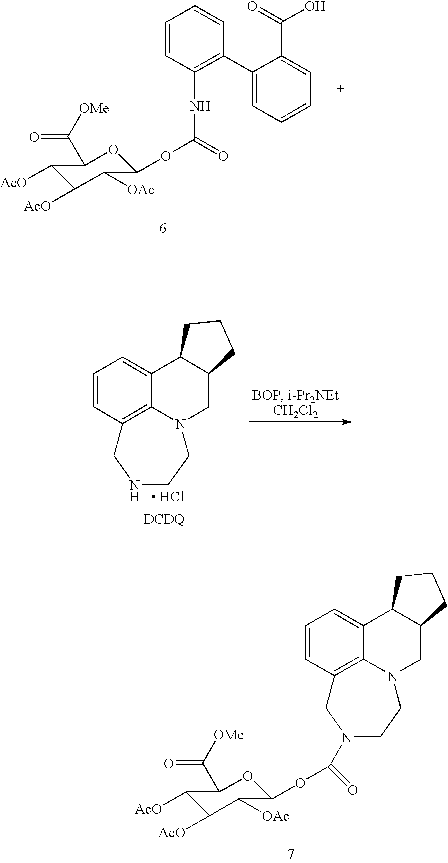

To a 500-mL flask was charged 3,4,5-Triacetoxy-6-(2′-carboxylbiphenyl-2-ylcarbamoyloxy)tetrahydropyran-2-carboxylic acid methyl ester 6 (5.0 g, 8.7 mmol), CH₂Cl₂ (200 mL) and BOP (4.2 g, 9.6 mmol, 1.1 eq). The mixture was stirred at room temperature under nitrogen atmosphere to become a solution. To this solution was added dropwise a solution of DCDQ (2.5 g, 9.6 mmol, 1.1 eq) and N,N-diisopropyl-N-ethyl amine (7.6 mL, 43.5 mmol, 5 eq) in CH₂Cl₂ (200 mL) in 10 min. The reaction mixture was stirred overnight and filtered through celite. The organic layer was washed with water (200 mL), dried over MgSO₄ and evaporated. The residue was purified by column chromatography (column: 4.5×50 cm, silica gel: 500 g, solvent: EtOAc/heptane (2/8, 4 L), (3/7, 8 L), 50 mL/fraction) to obtain compound 7 (4.0 g, HPLC: 74%), further slurry in CH₂Cl₂ to give compound 7 (3.52 g, 68.8%, HPLC: 96%). ¹HNMR (300 MHz, DMSO-d6): 7.12-7.08 (m, ¹H), 6.98-6.96 (m, ¹H), 6.86-6.77 (m, ¹H), 5.81, 5.79 (2d, J=8.1 Hz, ¹H, β-anomer), 5.10-4.90 (m, 2H), 4.63-4.36 (m, 2H), 4.17-4.12 (m, ¹H), 3.88-3.68 (m, ¹H), 3.64, 3.59 (2s, 3H), 3.40-3.21 (m, ¹H), 3.04-2.59 (m, 4H), 2.30-2.14 (m, ¹H), 2.05-1.95 (3s, 9H), 1.70-1.20 (m, 5H). MS: m/z 589 [M+H]⁺.

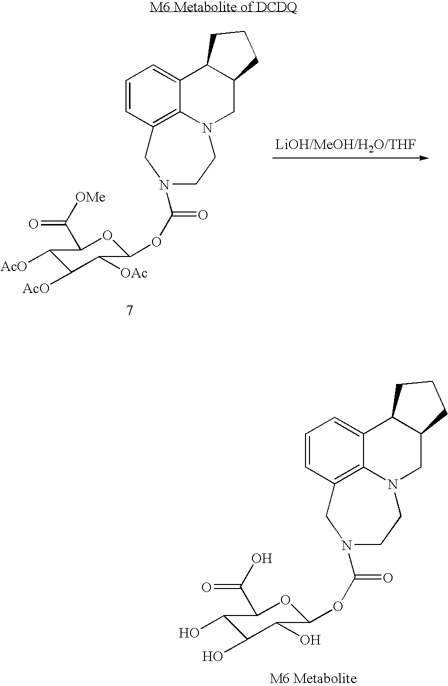

A solution of compound 7 (5.0 g, 8.5 mmol) in THF (64 mL) was added MeOH (319 mL) and H₂O (70 mL). The solution was cooled to 0-5° C. (ice-water bath). And a solution of LiOH.H₂O (2.1 g, 51 mmol, 6 eq) in H₂O (58 mL) [0.1 N LiOH/MeOH/THF/H₂O] was added dropwise in 20 min. The reaction mixture was stirred at 0-5° C. for 2 hours under N₂ atmosphere. Progress of the deprotection was monitored on reversed-phase TLC (SiO₂—C₁₈ MeCN/H₂O, 3/7). The reaction mixture was diluted with H₂O (500 mL) and neutralized by adding HOAc (3.1 g, 51 mmol) at 20° C. The solvent was concentrated under reduced pressure at 22° C. and the resultant aqueous suspension was lyophilized to give crude M6 metabolite (6.2 g, 100%). Further purification of the crude compound (1.2 g) using Biotage silica gel column chromatography (Horizon),2 CHCL₃/MeOH/H₂O as an eluent provided M6 (400mg) with 95% purity (HPLC). ¹HNMR (300 MHz, DMSO-d6, D₂O exchange): 7.13-6.99 (m, 2H), 6.87-6.80 (m, ¹H), 5.09 (d, J=7.8 Hz, ¹H, β-anomer), 4.77-4.58 (m, ¹H), 4.19-4.12 (m, ¹H), 3.93 (m, ¹H), 3.40-2.87 (9m, 9H), 2.68-2.60 (m, ¹H), 2.24-1.99 (m, 3H), 1.63-1.20 (m, 4H); 13C (75 MHz, DMSO-d6): 173.3, 173.1, 154.6, 154.0, 147.3, 132.5, 132.4, 130.9, 130.6, 130.1, 127.9, 127.7, 121.4, 121.1, 96.9, 96.3, 77.1, 76.9, 75.3, 73.2, 72.9, 72.6, 56.9, 56.1, 55.6, 50.9, 50.4, 48.7, 41.7, 35.0, 34.9, 32.5, 32.3, 29.8, 24.1; LC/MS (ESI), m/z 449 [M+H]⁺.

### REFERENCES

1. HPLC equipment: Waters 2690
Sample preparation: add 2-3 drops of the reaction mixture to 2 mL of acetonitrile, shake well to a solution and subject to HPLC analysis.
HPLC conditions:
Column: Alltima C₁₈ 3 μm 7×53 mm
Column temperature: 25° C.
Mobile phases: Solvent A=1900 mL H₂O, 100 mL CH₃CN, 1 mL H₃PO₄;

Solvent B=1900 mL CH₃CN, 100 mL H₂O, 1 mL H₃PO₄

TABLE 1
W2690 Gradient table

| | Flow | | | |
|---|---|---|---|---|
| Time | Rate | | | |
| (min.) | (mL/min) | % A | % B | curve |
| | 2.50 | 100 | 0 | |
| 2 | 2.50 | 100 | 0 | 6 |
| 9 | 2.50 | 0 | 100 | 6 |
| 11 | 2.50 | 0 | 100 | 6 |
| 12 | 2.50 | 100 | 0 | 6 |
| 16 | 2.50 | 100 | 0 | 6 |

UV: 215 nm
Injection volume: 10 μL
2. Biotage Flash-12 (Horizon) Method for the Final Purification of M6
Mobile phases:
A: CHCl₃:MeOH:H₂O (8:2:0.2)
B: CHCl₃:MeOH:H₂O (7:3:0.5)
Gradient by Column Volume (CV, 120 mL/CV):
2 (CV): A 100%
5 (CV): A 100%→B 100%
3 (CV): B 100%
Sample loading: Dissolve 1.2 g of the crude M6 in 8 mL of mobile phase A and pour into the samplet.
Flow rate: 40 mL/min
UV: 254 nm
Fraction: 12 mL/fraction, total 96 fractions

### In Vitro/In Vivo Metabolite Studies

DCDQ is a potent 5-HT_{2C} agonist and is effective in several animal models predictive of antipsychotic activity, with an atypical antipsychotic profile. The behavioral profile of DCDQ in these models is consistent with atypical antipsychotic-like activity with diminished extrapyramidal side-effect liability. The 5-HT_{2C} agonist DCDQ may also be effective in treating the mood disorders or the cognitive impairments associated with schizophrenia.

Several metabolites of DCDQ were identified through in vivo and in vitro models. Without being bound to the theory behind the pathways, FIGS. 1-4 show proposed metabolic pathways leading to these compounds.

### In Vitro Metabolism of [¹⁴C]DCDQ in Liver Microsomes of Mice, Rats, Dogs and Humans, and in Cryopreserved Human Hepatocytes

The metabolism of [¹⁴C]DCDQ was investigated by incubation with liver microsomes from male and female CD-1 mice, Sprague Dawley rats, beagle dogs and human liver microsomes pooled across sexes, and cryopreserved male human hepatocytes. Using human liver microsomes, the Kₘ values for the formation of the major oxidative metabolite M1 and the carbamoyl glucuronide M6 were 10.8 and 56.1 μM, respectively.

Species differences were observed in DCDQ metabolism. Oxidative metabolism was the major metabolic pathway for DCDQ in hepatic microsomal incubations. Several hydroxy metabolites (M1, M2, M3 and M4) of DCDQ were detected with human liver microsomes in the presence of NADPH. Metabolite M1 was not detected in other species. Metabolites M2 and M3 were also observed with dog and rat. Metabolite M4 was also detected in rat, but not in mouse or dog. Mouse appeared to have less extensive metabolism than other species, and M2 was the only metabolite detected with mouse liver microsomes. An N-oxide of DCDQ imine (M5) was detected with dog and human, but not with mouse or rat liver microsomes. Formation of DCDQ imine (P3) and currently unidentified products P1 and P2 in liver microsomes from all species was not NADPH-dependent, and requires further investigation. Sex differences were not observed in microsomal incubations for mice, rats and dogs.

In the presence of UDPGA, the carbamoyl glucuronide of DCDQ (M6) was detected with dog and human, but not with mouse or rat liver microsomes. While formation of the hydroxy metabolites was the major metabolic pathway with human liver microsomes in the presence of both NADPH and UDPGA, the carbamoyl glucuronide was the major metabolite in human hepatocytes at 50 μM DCDQ concentration.

In summary, DCDQ was converted to oxidative metabolites and a carbamoyl glucuronide in microsomal incubations and human hepatocytes.

### Introduction

This study investigated the in vitro biotransformation of DCDQ in liver microsomes and human hepatocytes. Cytochrome P450 and UDP-glucuronosyltransferase dependent pathways were examined and DCDQ metabolites were characterized by LC/MS.

### Materials and Methods

### Materials

[¹⁴C]DCDQ hydrochloride (Lot L25073-42) was synthesized by the radio-synthesis group of Wyeth Research (Pearl River, N.Y.). The radiochemical purity of [¹⁴C]DCDQ was 98.9% and the chemical purity was 99.9% by UV detection. The specific activity of the [¹⁴C]DCDQ was 222.9 μCi/mg as a hydrochloride salt. The chemical structure of [¹⁴C]DCDQ is shown with the position of the ¹⁴C label. The non-labeled DCDQ hydrochloride (Lot PB3312) with a chemical purity of 98.6% was synthesized by Wyeth Research (Pearl River, N.Y.). Unless otherwise indicated, when referring to DCDQ or [¹⁴C]DCDQ, the hydrochloride salt is assumed.

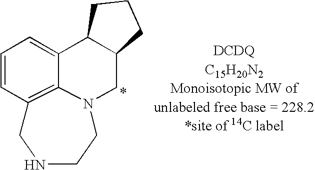

Cryopreserved human hepatocytes, hepatocyte suspension media and hepatocyte culture media were obtained from In Vitro Technologies (Baltimore, Md.). The hepatocytes were from two male individuals (Lot 070, 57 year old and Lot DRL, 44 year old) with testosterone 6β-hydroxylase activity of 55 and 43 pmol/106 cells/min, respectively, as determined by In Vitro Technologies. Liver microsomes listed in the following Table 2 from CD-1 mice, Sprague Dawley rats and beagle dogs were also obtained from In Vitro Technologies.

TABLE 2
Characteristics Of Mouse, Rat And Dog Liver Microsomes

| | | | | P450 Content |
|---|---|---|---|---|
| | | | Number of Animals for | (nmol/mg |
| Species | Sex | Lot No. | Pool | protein) |
| Mouse | Male | 100005 | 20 | 0.40 |
| | Female | 100005 | 18 | 0.54 |
| Rat | Male | 111 | 23 | 0.79 |
| | Female | 108 | 50 | 0.55 |
| Dog | Male | M100006 | 5 | 0.57 |
| | Female | 108 | 4 | 0.43 |

Human liver microsomes from subjects 3, 6, 15, 17, 18 and 19 were prepared from livers received from IIAM (Exton, Pa.). These microsomes were prepared and characterized by Dr. Andrew Parkinson and are described in Parkinson A. Preparation and characterization of human liver microsomes. Wyeth-Ayerst Research GTR-25617, 1994, which is hereby incorporated by reference. Microsomal preparations were stored at approximately −70° C. in aliquots of 250-500 μL until use. The following Table lists the characteristics of the human liver microsomes used in this study.

TABLE 3
Characteristics Of The Individual Human Liver Microsomes

| Individual | | | P450 Content |
|---|---|---|---|
| Number | Date Prepared | Sex | (nmol/mg protein) |
| 3 | Mar. 12, 1993 | F | 0.52 |
| 6 | Mar. 15, 1993 | M | 0.56 |
| 15 | Mar. 19, 1993 | F | 0.53 |
| 17 | Mar. 29, 1993 | F | 0.38 |
| 18 | Mar. 29, 1993 | M | 0.36 |
| 19 | Mar. 29, 1993 | F | 0.71 |

| | |
|---|---|
| Mean value of pooled microsomes | 0.51 |
| (N = 6) | |

Ultima Gold, Ultima Flo, Permafluor E+-scintillation cocktails, and Carbo-Sorb E carbon dioxide absorber were purchased from Perkin Elmer (Wellesley, Mass.). High performance liquid chromatography (HPLC) grade water and acetonitrile were obtained from EMD Chemicals (Gibbstown, N.J.). Uridine 5′-diphosphoglucuronic acid triammonium salt (UDPGA) and EDTA were obtained from Sigma Chemical Co. (St. Louis, Mo.). Ammonium acetate and magnesium chloride were obtained from Mallinckrodt Baker Inc. (Phillipsburg, N.J.). All other reagents were analytical grade or better.

### Methods

### Incubation of [¹⁴C]DCDQ with Liver Microsomes of Mice, Rats, Dogs and Humans

[¹⁴C]DCDQ was mixed with non-radiolabeled DCDQ (1:3 or 1:5) in the incubations. Microsomal incubations consisted of [¹⁴C]DCDQ, magnesium chloride (10 mM) and liver microsomes incubated in 0.5 mL of 0.1 M potassium phosphate buffer, pH 7.4. [¹⁴C]DCDQ (20 μL) in water was added to the incubation tubes containing buffer, magnesium chloride solution and microsomes. After mixing, the tubes were pre-incubated for 2 minutes in a shaking water bath at 37° C. The reactions were initiated by the addition of UDPGA or the NADPH regenerating system. UDPGA was added to incubations as a 50 μL aliquot of a 20 mM solution in water, to give a final concentration of 2 mM. An NADPH regenerating system (30 μL) was added to incubations to evaluate CYP450-mediated metabolism. The NADPH regenerating system consisted of glucose-6-phosphate (2 mg/mL), glucose-6-phosphate dehydrogenase (0.8 units/mL) and NADP⁺ (2 mg/mL). Control incubations were conducted under the same conditions, but without the NADPH generating system, UDPGA or microsomes. All incubations were performed in duplicate. Incubations were stopped by the addition of 0.5 mL ice-cold methanol. Samples were vortex-mixed. Denatured proteins were separated by centrifugation at 4300 rpm and 4° C. for 10 minutes (Model T21 super centrifuge, Sorvall). The protein pellets were extracted with 0.5 mL of methanol. The supernatant was combined for each sample, mixed and evaporated to a volume of about 0.3 mL under a nitrogen stream in a Zymark TurboVap LV evaporator (Caliper Life Science, Hopkinton, Mass.). The concentrated sample was centrifuged and aliquots were radioassayed and analyzed by HPLC. This method recovered an average of 92.1% of the radioactivity from the reaction mixture.

Initial rate conditions were determined for DCDQ metabolism in incubations with human liver microsomes in the presence of NADPH or UDPGA. Incubations for the time course study contained 20 μM of [¹⁴C]DCDQ and 0.5 mg/mL of microsomal proteins, and were incubated at 37° C. with mild shaking for 0, 5, 10, 20, 30, 40, 50 and 60 minutes. The protein dependence study was conducted with 20 μM of [¹⁴C]DCDQ incubated for 20 minutes with 0, 0.1, 0.25, 0.5, 0.75 and 1.0 mg/mL of microsomal proteins.

The Kₘ values were determined with 0.5 mg/mL of human liver microsomes incubated with [¹⁴C]DCDQ for 20 minutes with the NADPH regenerating system or for 10 minutes with UDPGA. [¹⁴C]DCDQ concentrations used were 0.5,1, 5, 10, 25, 50, 75 and 100 μM.

To evaluate species differences in cytochrome P450- and UGT-mediated metabolism, [¹⁴C]DCDQ was incubated for 20 minutes with 0.5 mg/mL liver microsomal proteins from mice, rats, dogs or humans in the presence of the NADPH regenerating system or UDPGA. The assay conditions were the same as described above, and DCDQ concentrations were 12 μM and 56 μM for cytochrome P450- and UGT-mediated metabolism, respectively.

Samples were analyzed for metabolites by radioactivity flow detection and by LC/MS.

### Preparation of Human Hepatocytes

Vials containing cryopreserved human hepatocytes were thawed in a 37° C. water bath with gentle shaking until the ice was almost melted. The vials were removed from the water bath and gentle shaking continued at room temperature for 30-60 seconds until completely thawed. The hepatocyte suspensions from each vial were immediately transferred to pre-cooled 50 mL beakers on ice. To each beaker, 12 mL of ice-cold hepatocyte suspension media was added dropwise over one minute, with occasional, gentle shaking by hand to prevent the cells from settling. The cell suspensions were transferred to a 15 mL tube and centrifuged at 100 g force for 3 min at 4° C. (Model T21 super centrifuge, Sorvall). The supernatant was discarded and the pellets were re-suspended in 4 mL of ice-cold hepatocyte culture media. The cell suspensions contained approximately 3.1×106 viable hepatocytes/mL. The average viability was 76.0% as determined using Trypan Blue exclusion and a hemacytometer.

Incubation of [¹⁴C]DCDQ with Human Hepatocytes

The cell suspensions were distributed into 12-well plates at 1.0 mL per well. Incubations were performed using pooled hepatocytes from two donors. [¹⁴C]DCDQ in water was added to the hepatocyte suspension at a final concentration of 10 or 50 μM. Incubations were carried out at 37° C. for 4 hours in an incubator supplied with 5% CO₂. At the end of the incubation, the reaction was stopped by the addition of 200 μL cold methanol to each well. The content of each well was transferred to a 15 mL centrifuge tube and sonicated for 30 seconds. After vortex mixing with 6 mL methanol and then centrifugation, the supernatant was transferred to a clean tube and evaporated to about 0.5 mL in a TurboVap evaporator. The residue was analyzed by HPLC and LC/MS.

HPLC Analysis

A Waters model 2690 HPLC system (Waters Corp., Milford, Mass.) with a built-in autosampler was used for analysis. Separations were accomplished on a Phenomenex Luna C₁₈(2) column (2×150 mm, 5 μm) (Phenomenex, Torrance, Calif.) coupled with a filter (4×2 mm) cartridge. A variable wavelength UV detector set to monitor 250 nm and Flo-One β Model A525 radioactivity flow detector (Perkin Elmer) with a 250 μL LQTR flow cell were used for data acquisition. The flow rate of Ultima Flow M scintillation fluid was 1 mL/min, providing a mixing ratio of scintillation cocktail to mobile phase of 5:1. The sample chamber in the autosampler was maintained at 4° C., while the column was at ambient temperature of about 20° C. The mobile phase consisted of 10 mM ammonium acetate, pH 4.5 (A) and methanol (B) and was delivered at 0.2 mL/min. The linear gradient conditions were as follows:

TABLE 4

| Time (min) | A (%) | B (%) |
|---|---|---|
| 0 | 90 | 10 |
| 3 | 90 | 10 |
| 25 | 60 | 40 |
| 45 | 15 | 85 |
| 50 | 15 | 85 |

### Liquid Chromatography/Mass Spectrometry

An Agilent Model 1100 HPLC system (Agilent Technologies, Palo Alto, Calif.) including an autosampler and diode array UV detector was used for LC/MS analysis. The UV detector was set to monitor 200 to 400 nm. For selected LC/MS analysis, radiochromatograms were acquired using a β-Ram model 3 radioactivity flow detector (IN/US Systems Inc., Tampa, Fla.) equipped with a solid scintillant flow cell. Separations were accomplished on a Phenomenex Luna C18(2) column (2×150 mm, 5 μm) under the same conditions as described above.

The mass spectrometer used for metabolite characterization was a Micromass Q-TOF-2 quadrupole time-of-flight hybrid mass spectrometer (Nature Corp.). The mass spectrometer was equipped with an electrospray ionization (ESI) interface and operated in the positive ionization mode. Collision energy settings of 5 and 30 eV were used for full MS and MS/MS scans, respectively. Settings for the mass spectrometer are listed below.

TABLE 5
Micromass Q-TOF-2 Mass Spectrometer Settings

| Capillary Voltage | 3.0 kV |
|---|---|
| Cone | 30 V |
| Source Block Temperature | 100° C. |
| Desolvation Gas Temperature | 250° C. |
| Desolvation Gas Flow | 550 L/hr |
| Cone Gas Flow | 50 L/hr |
| CID Gas Inlet Pressure | 13-14 psig |
| TOF-MS resolution (m/Δm) | 8000 |

### Data Analyses

Flo-One analytical software (Perkin Elmer, version 3.6) was utilized to integrate the radioactive peaks. The computer program Microsoft Excel® 97 was used to calculate means and standard deviations and to perform the Student t-test. Micromass MassLynx software (Waters, version 4.0) was used for collection and analysis of LC/MS data.

### Results

### Determination of Kₘ Values with Human Liver Microsomes

Initial rate conditions and Km values for metabolite formation from [¹⁴C]DCDQ were determined for human liver microsomes. In the time-dependency studies, NADPH-dependent formation of the major oxidative metabolites (M1, M2, M3 and M4) was linear for 20 minutes and formation of the carbamoyl glucuronide (M6) was linear for 10 minutes (data not shown). In the protein-dependency studies, oxidative metabolism and carbamoyl glucuronide formation were linear up to 0.5 mg/mL microsomal protein. The Kₘ values for the formation of the major oxidative metabolite M1 and the carbamoyl glucuronide M6 in human liver microsomes were 10.8 and 56.1 μM, respectively. The Kₘ values for formation of metabolites M2, M3 and M4 in human liver microsomes ranged from 8.9 to 13.8 μM. The Kₘ value for metabolite M5 formation in human liver microsomes was 36.2 μM.

### [¹⁴C]DCDQ Metabolism by Liver Microsomes of Mice, Rats, Dogs, and Humans

For species comparison in microsomal incubations, DCDQ concentrations for P450- and UGT-mediated metabolism were 12 and 56 μM, respectively, which were about the Kₘ values. In the presence of the NADPH regenerating system, four hydroxy metabolites (M1, M2, M3 and M4) were detected with human microsomes. Metabolite M1 was not detected in other species. Metabolites M2 and M3 were observed with dog and rat. Metabolite M4 was also detected in rat, but not in mouse or dog. Mouse appeared to have less extensive metabolism than other species, and M2 was the only metabolite detected with mouse liver microsomes. An N-oxide of DCDQ imine (M5) was detected with dog and human, but not mouse or rat. Three other peaks (P1, P2 and a DCDQ imine P3) were also observed in microsomal incubations. Formation of P1, P2 and P3 were not NADPH-dependent. Since these products were not formed in the control incubations without microsomes (data not shown), their formation may be catalyzed by non-P450 enzymes.

In the presence of UDPGA, formation of carbamoyl glucuronide of DCDQ (M6) was detected with liver microsomes of dog and human, but not mouse or rat. When DCDQ (20 μM) was incubated with human liver microsomes in the presence of both NADPH and UDPGA, formation of the hydroxy metabolites was the major metabolic pathway, and only minor amounts of the carbamoyl glucuronide were detected. Gender differences were not observed in microsomal incubations for mouse, rat and dog.

### [¹⁴C]DCDQ Metabolism by Human Hepatocytes

When DCDQ was incubated with human hepatocytes, the carbamoyl glucuronide (M6) was the most prominent metabolite at 50 μM DCDQ concentration. Oxidative metabolites were also observed at 50 μM DCDQ concentration, although less abundant relative to the carbamoyl glucuronide. Incubations containing 10 μM DCDQ with human hepatocytes produced oxidative metabolites at levels approaching those of the carbamoyl glucuronide. In addition to the metabolites formed in human microsomal incubations, another metabolite (M7) was detected. The DCDQ imine (P3), which was formed in microsomes, was also observed in the hepatocyte incubations.

### Metabolite Characterization by LC/MS Analysis

Mass spectra were obtained by LC/MS and LC/MS/MS analysis for DCDQ and its metabolites. Structural characterization of these compounds is summarized in Table 6.

TABLE 6
Metabolites Of Dcdq Generated By Liver
Microsomes And Hepatocytes

| Products | [M + H]⁺ | Site of Metabolism | Source |
|---|---|---|---|
| M1, hydroxy DCDQ | 245 | Diazepane ring | HM, HH |
| M2, hydroxy DCDQ | 245 | Pyridine ring | MM, RM, DM, HM, HH |
| M3, hydroxy DCDQ | 245 | Cyclopentane ring | MM, RM, DM, HM, HH |
| M4, hydroxy DCDQ | 245 | Cyclopentane ring | MM, RM, DM, HM, HH |
| M5, DCDQ imine oxide | 243 | Diazepane ring | DM, HM, HH |
| M6, carbamoyl glucuronide | 449 | Diazepane ring | DM, HM, HH |
| of DCDQ | | | |
| M7, keto DCDQ | 243 | Pyridine or | HH |
| | | cyclopentane ring | |
| P3, DCDQ imine | 227 | Diazepane ring | MM, RM, DM, HM, HH |
| DCDQ | 229 | | MM, RM, DM, HM, HH |

MM: Mouse liver microsomes
RM: Rat liver microsomes
DM: Dog liver microsomes
HM: Human liver microsomes
HH: Human hepatocytes

The mass spectral characterization of DCDQ and its metabolites identified in each of the studies are discussed further below.

### Discussion

Species differences were observed in DCDQ metabolism. Oxidative metabolism was the major metabolic pathway for DCDQ in hepatic microsomal incubations. Several hydroxy metabolites (M1, M2, M3 and M4) of DCDQ were detected with human liver microsomes in the presence of NADPH (FIG. 1). Metabolites M2 and M3 were also observed with dog and rat liver microsomes. Mouse had less extensive metabolism than other species, and M2 was the only metabolite detected with mouse liver microsomes. An N-oxide of DCDQ imine (M5) was detected in microsomal incubations for dog and human, but not mouse or rat liver microsomes. In the presence of UDPGA, the carbamoyl glucuronide of DCDQ (M6) was detected with dog and human, but not mouse or rat. While formation of the hydroxy metabolites was the major metabolic pathway with human liver microsomes in the presence of both NADPH and UDPGA, the carbamoyl glucuronide M6 was the major metabolite in human hepatocytes at 50 μM DCDQ concentration. Enzyme systems other than P450 may also contribute to DCDQ metabolism by formation of a DCDQ imine (P3) and other products (P1 and P2). Formation of products P1, P2 and P3 was not NADPH-dependent, and requires further investigation since they were generally present in all incubations with liver microsomes and hepatocytes. Sex differences were not observed for mice, rats or dogs in microsomal incubations.

In summary, DCDQ was converted to oxidative metabolites and a carbamoyl glucuronide in microsomal incubations and human hepatocytes.

### In Vivo Metabolism of [¹⁴C]DCDQ in Male and Female Sprague-Dawley Rats Following a Single (5 mg/kg) Oral Gavage Administration

Synopsis

The present study investigated the in vivo metabolism of [¹⁴C]DCDQ in male and female Sprague-Dawley rats after a single oral administration (5 mg/kg). Blood, plasma and brain were collected at 2, 4, 8 and 24 hour post-dose from male rats and at 2 and 8 hour post-dose from female rats. Urine and feces were collected from male rats at intervals of 0-8 and 8-24 hours post-dose.

In male rats, plasma radioactivity concentrations were 632±144, 659±16.5, 465±43.1, and 46.9±8.30 ng equivalents/mL at 2, 4, 8 and 24 hour post-dose, respectively. For female rats, the mean plasma radioactivity concentration of 658±189 ng equivalents/mL at 2 hour post-dose was similar to male rats, but the average radioactivity concentration of 338±60.7 ng equivalents/mL at 8 hour post-dose was lower than male rats. The average blood-to-plasma ratio was about 1.1 between 2 and 8 hour post-dose, indicating limited partitioning of DCDQ and its metabolites into blood cells.

DCDQ represented an average of 13% to 20% of plasma radioactivity between 2 and 8 hour post-dose. The 24 hour plasma samples were not analyzed for profiles due to low radioactivity concentrations. Changes in metabolite profiles were not apparent over time. Metabolites detected in plasma included hydroxy DCDQ metabolites (M1, M2, M3, M4 and M10), keto DCDQ (M7), and the phase II metabolites DCDQ sulfamate (M12), di-dehydro DCDQ sulfamate (M14), hydroxy DCDQ sulfates (M8 and M13), hydroxy DCDQ glucuronide (M9) and acetylated hydroxy DCDQ (M11). Plasma metabolite profiles exhibited sex-related differences. While the hydroxy DCDQ metabolites (M1, M2 and M3), the keto DCDQ (M7) and the hydroxy DCDQ glucuronide (M9) were the major metabolites in male rat plasma, the hydroxy DCDQ metabolite (M3), the hydroxy DCDQ sulfate (M8), the hydroxy DCDQ glucuronide (M9) and DCDQ sulfamate (M12) were the major metabolites in female rats. The primary sex difference was in the formation of sulfates or sulfamates.

Urinary excretion was a major route of elimination of orally administered DCDQ and accounted for 66.7% of the dose. The major metabolites observed in plasma samples were also detected in urine, where DCDQ accounted for less than 1% of the dose. The hydroxy metabolites (M1 and M3), the keto DCDQ (M7) and the glucuronide (M9) were the major metabolites in urine. An average of 21.1% of the dosed radioactivity was recovered in feces. Metabolites M3, M8, M9, M10, M11 and only trace amounts of DCDQ were detected in male rat feces.

Radioactivity in brain tissue was significantly higher than in plasma at 2, 4 and 8 hour post-dose. Brain radioactivity concentrations were 5.12±1.28, 4.94±0.44, 3.25±0.99 and 0.037±0.002 pg equivalents/g tissue at 2, 4, 8 and 24 hour post-dose for male rats, respectively, while concentrations were 6.38±2.22 and 2.85±0.68 μg equivalents/g tissue at 2 and 8 hour post-dose for female rats, respectively. The average brain-to-plasma radioactivity ratios between 2 and 8 hour post-dose ranged from 6.9 to 9.6, indicating significant uptake by brain tissue. By 24 hour post-dose, the average brain-to-plasma radioactivity ratio decreased to 0.8. DCDQ accounted for an average of greater than 90% of brain radioactivity for male and female rats between 2 and 8 hour post-dose. Based on the radioactivity concentrations and chromatographic distribution of brain radioactivity, it was estimated that the average brain-to-plasma DCDQ ratios ranged from 49.9 to 56.1. There were no significant gender differences or changes over time between 2 and 8 hour post-dose. Only minor amounts of metabolites M1, M3, M7, M10 and M11 were detected in male or female rat brain. These data indicated that DCDQ readily crossed the blood brain barrier, while uptake of metabolites into brain tissue was limited. The brain-to-plasma radioactivity ratios also suggested that clearance from brain occurred rapidly after 8 hour post-dose, since the ratios decreased from 6.9 to 0.8 by 24 hour post-dose.

In summary, DCDQ was extensively metabolized in rats to predominantly oxidative metabolites. Plasma profiles for male and female rats differed in sulfate and sulfamate conjugates of DCDQ and its oxidative metabolites. DCDQ was the predominant drug related component in brain while only minor amounts of metabolites were observed, and gender difference was not apparent. DCDQ readily crossed the blood brain barrier while uptake of metabolites was limited to minor amounts of oxidative metabolites.

### Introduction

A previous mass balance study showed that urine was the major route of excretion in rats, with an average of 64.3% of the dosed radioactivity recovered in urine. An in vitro study with liver microsomes showed that oxidative metabolism was the major metabolic pathway for DCDQ in rats. (Iwasaki K, Shiraga T, Tada K, Noda K, Noguchi H. Age- and sex-related changes in amine sulphoconjugation in Sprague-Dawley strain rats. Comparison with phenol and alcohol sulphoconjugations. Xenobiotica. 1986;16:717-723.) The present study investigated the metabolism of [¹⁴C]DCDQ in rats following a single 5 mg/kg oral dose.

### Materials and Methods

### Materials

[¹⁴C]DCDQ hydrochloride was synthesized by the radiosynthesis group of Wyeth Research (Pearl River, N.Y.) as described in the in vitro study discussed above. Ultima Gold, Ultima Flo, Permafluor E+-scintillation cocktails, and Carbo-Sorb E carbon dioxide absorber were purchased from Perkin Elmer (Wellesley, Mass.). Polysorbate 80 was obtained from Mallinckrodt Baker (Phillipsburg, N.J.) and methylcellulose was from Sigma-Aldrich (Milwaukee, Wis.). Solvents used for extraction and for chromatographic analysis were HPLC or ACS reagent grade from EMD Chemicals (Gibbstown, N.J.).

### Methods

### Drug Administration and Specimen Collection

Dose preparation, animal dosing, and specimen collection were performed at Wyeth Research, Collegeville, Pa. The dose vehicle contained 2% (w/w) Tween 80 and 0.5% methylcellulose in water. On the day of dosing, [¹⁴C]DCDQ (12.2 mg) and non-labeled DCDQ (36.5 mg) were dissolved in the vehicle to a final concentration of approximately 2 mg/mL.

Male rats weighing from 318 to 345 grams and female rats weighing from 227 to 255 grams at the time of dosing were purchased from Charles River Laboratories, Wilmington, Mass. Non-fasted rats were given a single 5 mg/kg (˜300 μCi/kg) dose of DCDQ at a volume of 2.5 mL/kg via intragastric gavage. Animals were provided Purina rat chow and water ad libitum, and were kept individually in metabolism cages. Male rats were sacrificed at 2, 4, 8 and 24 hour after dose administration. Female rats were sacrificed at 2 and 8 hour after dose administration.

At sacrifice, blood samples were collected by cardiac puncture into tubes containing EDTA as the anticoagulant and placing them on ice. Aliquots of 50 μL were removed for combustion and determination of radioactivity content. Plasma was immediately obtained from the remaining blood by centrifugation at 4° C. Brains were excised after perfusion with 50 mL of chilled sterile saline. Urine samples were collected on dry ice at intervals of 0-8 and 8-24 hour post-dosing. Feces were collected at intervals of 0-8 and 8-24 hr post-dosing at room temperature and were homogenized as described previously. The biological specimens and aliquots of the dosing solution at pre- and post-dose were stored at approximately −70° C. until analyzed.

### Radioactivity Determinations

Plasma (20 μL) and urine (50 μL) aliquots were analyzed for radioactivity concentrations. Radioactivity determinations of dose, plasma and urine were made with a Tri-Carb Model 3100 TR/LL liquid scintillation counter (LSC) (Perkin Elmer) using 10 mL of Ultima Gold as the scintillation fluid.

Brain and fecal samples were weighed and homogenized in water at volume-to-weight ratios of about 1:1 and 5:1, respectively. Blood aliquots (50 μL), brain homogenates (0.1 gram) and fecal homogenates (0.2 gram) were placed on Combusto-cones with Combusto-pads and combusted. A model 307 Tri-Carb Sample Oxidizer, equipped with an Oximate-80 Robotic Automatic Sampler (Perkin Elmer), was used for combustion. The liberated ¹⁴CO₂ was trapped with Carbo-Sorb E carbon dioxide absorber, mixed with PermaFluor® E+ liquid scintillation cocktail, and counted in a Tri-Carb Model 3100 TR/LL liquid scintillation counter (Perkin Elmer). The oxidation efficiency of the oxidizer was 98.2%.

A Flo-One β Model A525 radioactivity detector (Perkin Elmer) with a 250 μL LQTR flow cell was used for in-line radioactivity detection for HPLC. The flow rate of Ultima Flow M scintillation fluid was 1 mumin, providing a mixing ratio of scintillation cocktail to mobile phase of 5:1. The limits of detection were approximately 1 ng equivalent/g for brain, 2 ng equivalents/mL for plasma, 5 ng equivalents/g for feces and 10 ng equivalents/mL for urine.

### Dose Analysis

Aliquots of the pre- and post-dose solutions were diluted with 25% methanol in water and analyzed for radioactivity concentrations as described above. Approximately 100,000 DPM in 10 μL of the diluted solution was analyzed by HPLC for radiochemical purity and chemical purity. To determine the specific radioactivity of the dose suspension, non-radiolabeled DCDQ was dissolved in methanol, diluted with 25% methanol in water, and concurrently analyzed by HPLC to generate a standard curve. Aliquots (10 μL) of the diluted dose solution were injected onto the HPLC column and fractions were collected at 1 minute intervals after UV detection. Radioactivity in each fraction was determined. Fractions were also collected from a blank injection to obtain the background level of radioactivity.

### Plasma Metabolite Profiles

Plasma samples were analyzed for metabolite profiles by HPLC. Aliquots of 1.5 mL plasma were mixed with 3.0 mL methanol, placed on ice for about 10 minutes, and then centrifuged. The supernatant was transferred to a clean tube. The protein pellets were extracted once with 3.0 mL methanol. The supernatants from precipitation and extraction of each sample were pooled, mixed, and evaporated at 22° C. under nitrogen in a Zymark TurboVap LV (Caliper Life Sciences, Hopkinton, Mass.) to about 0.3 mL. The concentrated extract was centrifuged, the supernatant volume measured and extraction efficiency was determined by analyzing duplicate 10 μL aliquots for radioactivity. An aliquot of the supernatant (50-200 μL) was analyzed by HPLC with radioactivity flow detection. Plasma extracts were also analyzed by LC/MS.

### Analysis of Feces and Urine

Fecal homogenates were analyzed for metabolite profiles. Aliquots of 1 gram of fecal homogenates were mixed with 2 mL methanol, placed on ice for about 10 minutes and centrifuged. The supernatant was transferred to a clean tube. The residue was extracted three times with 2 mL of a water:methanol (3:7) mixture. The supernatants of each sample were combined, evaporated to about 1 mL, and centrifuged. Extraction efficiency was determined by analyzing aliquots of 10 μL of the supernatant for radioactivity. An aliquot (50-200 μL) of the supernatant was analyzed by HPLC with radioactivity flow detection for profiling. Samples were also analyzed by LC/MS to characterize the radioactive peaks.

Urine was analyzed for radioactivity concentration as and analyzed for metabolite profiles by direct injection onto the HPLC column. LC/MS analyses for metabolite identification were also carried out with urine samples.

Metabolite Profiles in Brain

Brain homogenates were analyzed for metabolite profiles. Aliquots of 1 gram of brain homogenates were mixed with an equal volume of methanol, placed on ice for about 10 minutes and centrifuged. The supernatant was transferred to a clean tube. The residue was extracted three times with 1 mL methanol. The supernatants of each sample were combined, evaporated to about 0.5 mL, and centrifuged. Extraction efficiency was determined by analyzing aliquots of 10 μL of the supernatant for radioactivity. An aliquot (100-200 μL) of the supernatant was analyzed by HPLC with radioactivity flow detection for profiling. Samples were also analyzed by LC/MS to characterize the radioactive peaks.

### High Performance Liquid Chromatography

A Waters model 2690 HPLC system (Waters Corp., Milford, Mass.) with a built-in autosampler was used for analysis. Separations were accomplished on a Phenomenex Luna C₁₈(2) column (150×2.0 mm, 5 μm) (Phenomenex, Torrance, Calif.). The sample chamber of the autosampler was maintained at 4° C., while the column was at ambient temperature of about 20° C. A variable wavelength UV detector set to monitor 250 nm and a Flo-One β Model A525 radioactivity detector described above were used for data acquisition. The HPLC mobile phase consisted of 10 mM ammonium acetate, pH 4.5 (A) and methanol (B), and was delivered at 0.2 mL/min. Chromatographic condition A was used for dose analysis, while condition B was used for analysis of urine and plasma, brain and fecal extracts.

TABLE 7

| | | |
|---|---|---|
| Time (min) | A (%) | B (%) |

CONDITION A

| | | |
|---|---|---|
| 0 | 90 | 10 |
| 3 | 90 | 10 |
| 25 | 60 | 40 |

CONDITION B

| | | |
|---|---|---|
| 0 | 90 | 10 |
| 6 | 90 | 10 |
| 35 | 60 | 40 |
| 65 | 15 | 85 |
| 70 | 15 | 85 |

### LC/MS Analyses

An Agilent Model 1100 HPLC system (Agilent Technologies, Wilmington, Del.) including an autosampler and diode array UV detector was used for LC/MS analysis of plasma and urine samples. The UV detector was set to monitor 200 to 400 nm. For selected LC/MS analyses, radiochromatograms were acquired using a β-Ram model 3 radioactivity flow detector (IN/US Systems Inc., Tampa, Fla.) equipped with a solid scintillant flow cell. Fecal samples were also analyzed using a Waters Alliance model 2690 HPLC system. It was equipped with a built-in autosampler and a model 996 diode array UV detector set to 210-350 nm. The HPLC flow was split between a Radiomatic model 150TR flow scintillation analyzer (Perkin Elmer) and the mass spectrometer. Other HPLC conditions were the same as condition B described above.

The mass spectrometer used for metabolite characterization for plasma and urine was a Micromass Q-TOF-2 quadrupole time-of-flight hybrid mass spectrometer (Waters). The mass spectrometer was equipped with an electrospray ionization (ESI) interface and operated in the positive ionization mode. Collision energy settings of 5 and 30 eV were used for full MS and MS/MS scans, respectively. Settings for the mass spectrometer are listed below.

TABLE 8
MICROMASS Q-TOF-2 MASS SPECTROMETER SETTINGS

| Capillary Voltage | 3.0 Kv |
|---|---|
| Cone | 30 V |
| Source Block Temperature | 100° C. |
| Desolvation Gas Temperature | 250° C. |
| Desolvation Gas Flow | 550 L/hr |
| Cone Gas Flow | 50 L/hr |
| CID Gas Inlet Pressure | 13-14 psig |
| TOF-MS resolution (m/Δm) | 8000 |

A Micromass Quattro Micro mass spectrometer (Waters) was also used to analyze the fecal samples. It was equipped with an electrospray ionization (ESI) interface and operated in the positive ionization mode. Settings for the mass spectrometer are listed below.

TABLE 9
MICROMASS TRIPLE QUADRUPLE MASS SPECTROMETER
SETTINGS

| | |
|---|---|
| ESI spray | 2.75 KV |
| Cone | 30 V |
| MS1 Mass Resolution | 1-1.5 Da width at half height |
| MS2 Mass Resolution | 0.7 Da ± 0.2 Da width at half height |
| Desolvation gas flow | 875-950 L/hr |
| Cone Gas flow | 20-35 L/hr |
| Source block temp. | 80° C. |
| Desolvation gas temp. | 250° C. |
| Collision gas pressure | 1.3-1.5 × 10−3 mBar |
| Collision energy | 25 eV |

LC/MS/MS in the selected reaction-monitoring (SRM) mode (LC/SRM) was conducted with the triple quadruple mass spectrometer to monitor DCDQ and its metabolites in fecal samples. The transitions monitored are listed below.

TABLE 10

| Precursor Ion (m/z) | Product Ion (m/z) | Compound |
|---|---|---|
| 229 | 186 | DCDQ |
| 243 | 200 | M7 |
| 245 | 144 | M3, M4, and M10 |
| 245 | 184 | M3, M4, and M10 |
| 287 | 186 | M11 |
| 325 | 245 | M8, M13 |
| 421 | 245 | M9 |

### Data Analysis and Statistical Evaluation

Flo-One analytical software (Packard, version 3.6) was utilized to integrate the radioactive peaks. The computer program Microsoft Excel® 97 was used to calculate means and standard deviations and to perform the student t-test. Micromass MassLynx software (Waters, version 4.0) was used for collection and analysis of LC/MS data.

### Results

### Dose Analysis

The radiochemical purity and estimated chemical purity (by ultraviolet detection) of [¹⁴C]DCDQ in the dose solution were 99.0±0.3% and 99.6±0.1%, respectively. The pre- and post-dose aliquots had the same purity. The specific activity of [¹⁴C]DCDQ in the dosing solution was 48.2 μCi/mg as the hydrochloride salt. The average drug concentration was 2.48 mg/mL as the hydrochloride salt or 2.14 mg/mL as the free base. The actual dose of DCDQ administered ranged from 5.2 to 5.4 mg/kg as the free base, or 6.1 to 6.3 mg/kg as the hydrochloride salt.

### Plasma Radioactivity Concentrations and Metabolite Profiles

The concentrations of radioactivity in blood and plasma after a single oral dose of [¹⁴C]DCDQ are summarized in Table 11.

TABLE 11
Rat Blood And Plasma Concentrations (ng equivalents/mL) Of Radioactivity
Following A Single Oral 5 Mg/Kg Administration Of [¹⁴C]DCDQ

| | | | | |
|---|---|---|---|---|
| Time (hr) | Blood Individuals | Mean ± SD | Plasma Individuals | Mean ± SD |

Male

| | | | | | | | | |
|---|---|---|---|---|---|---|---|---|
| 2 | 606 | 624 | 888 | 706 ± 158 | 567 | 531 | 797 | 632 ± 144 |
| 4 | 698 | 725 | 689 | 704 ± 18.7 | 678 | 652 | 647 | 659 ± 16.5 |
| 8 | 538 | 475 | 562 | 525 ± 45.3 | 487 | 415 | 492 | 465 ± 43.1 |
| 24 | 66.5 | 58.9 | 78.7 | 68.0 ± 10.0 | 50.5 | 37.4 | 52.8 | 46.9 ± 8.30 |

Female

| | | | | | | | | |
|---|---|---|---|---|---|---|---|---|
| 2 | 661 | 932 | 499 | 697 ± 219 | 635 | 857 | 481 | 658 ± 189 |
| 8 | 413 | 292 | 406 | 307 ± 68.4* | 362 | 269 | 383 | 338 ± 60.7* |

*Significantly lower than male at 8 hour, p < 0.05

In male rats, the average plasma radioactivity concentrations were 632, 659, 465 and 46.9 ng equivalents/mL at 2, 4, 8 and 24 hour post-dose, respectively. In female rats, the average plasma radioactivity concentration of 658 ng equivalents/mL at 2 hour was similar to male rats, but the average plasma concentration of 338 ng equivalents/mL at 8 hour post-dose was significantly lower than in male rats. Blood samples had slightly higher radioactivity concentrations than plasma at all time points. The average blood-to-plasma radioactivity ratios ranged from about 1.1 for male and female rats at 2, 4 and 8 hour post-dose to about 1.5 for male rats at 24 hour post-dose, indicating limited partitioning of DCDQ or its metabolites into blood cells (Table 12).

TABLE 12
Rat Blood-To-Plasma Radioactivity Ratios Following A Single Oral
5 Mg/Kg Administration Of [¹⁴C]DCDQ

| | | |
|---|---|---|
| Time | Blood/Plasma Ratios | |
| (hr) | Individuals | Mean ± SD |

Male

| | | | | |
|---|---|---|---|---|
| 2 | 1.07 | 1.18 | 1.11 | 1.12 ± 0.06 |
| 4 | 1.03 | 1.11 | 1.07 | 1.07 ± 0.04 |
| 8 | 1.10 | 1.14 | 1.14 | 1.13 ± 0.02 |
| 24 | 1.32 | 1.58 | 1.49 | 1.46 ± 0.13 |

Female

| | | | | |
|---|---|---|---|---|
| 2 | 1.04 | 1.09 | 1.04 | 1.06 ± 0.03 |
| 8 | 1.14 | 1.08 | 1.06 | 1.09 ± 0.04 |

Plasma extracts contained an average of 82 to 96% of total plasma radioactivity for the 2, 4 and 8 hour samples. Metabolite profiles were not obtained from the 24 hour plasma samples due to low radioactivity concentrations. DCDQ was extensively metabolized in rats. The parent drug represented an average of 13 to 20% of total radioactivity in plasma extracts with no apparent differences between and females or over time (Tables 13 and 14). Several hydroxy DCDQ metabolites (M1, M2, M3, M4 and M10) and keto DCDQ (M7) were detected in plasma (FIG. 1). Phase II metabolites observed in plasma included DCDQ sulfamate (M12, major in female plasma only), di-dehydro DCDQ sulfamate (M14, major in female plasma only), hydroxy DCDQ sulfates (M8 and M13), hydroxy DCDQ glucuronide (M9) and acetylated hydroxy DCDQ (M11) (FIG. 1). Percent distribution of plasma radioactivity did not change significantly over time, except for metabolite M8, which was markedly lower at 8 hour post-dose. Metabolites M1, M2, M3, M7 and M9 were the major metabolites in male rats, while M3, M8, M9 and M12 were the major metabolites in female rats, indicating sex differences in metabolite profiles (Tables 13 and 14). A number of relatively minor metabolites detected in plasma extracts were not characterized, although when combined represented 19-38% of the plasma radioactivity. Plasma concentrations of the individual metabolites based on their percent distribution are presented in Table 4. DCDQ concentrations generally equaled or exceeded the concentrations of each individual metabolite in male and female rat plasma. In male rats, metabolites M1, M3+M9 and M7 exhibited the highest concentrations while in female rats, metabolites M3+M9 and M8 were the more prominent metabolites.

TABLE 13
Chromatographic Distribution (%) of Radioactivity in Rat Plasma Following Oral Administration of [¹⁴C]DCDQ (5 mg/kg)

| | | | | | | | | | | | | | |
|---|---|---|---|---|---|---|---|---|---|---|---|---|---|
| Time (hr) | M1 | M2 | M3 + M9 | M4 | M7 | M8 | M10 | M11 | M12 | M13 | M14 | DCDQ | Others^{a} |

Male

| | | | | | | | | | | | | | |
|---|---|---|---|---|---|---|---|---|---|---|---|---|---|
| 2 | 14 | 5.2 | 12 | 4.4 | 10 | 2.4 | 3.6 | 6.0 | | 1.9 | | 14 | 26 |
| | (2.7)^{b} | (2.3) | (4.8) | (0.85) | (1.3) | (1.5) | (0.83) | (2.2) | BQL^{c} | (0.65) | BQL | (4.9) | (4.8) |
| 4 | 9.1 | 3.4 | 16 | 4.3 | 9.4 | 4.6 | 1.5 | 5.2 | | 2.2 | | 14 | 31 |
| | (0.6) | (0.81) | (3.1) | (0.47) | (2.3) | (0.83) | (2.6) | (0.98) | BQL | (0.36) | BQL | (3.4) | (1.3) |
| 8 | 8.9 | 4.3 | 14 | 2.5 | 12 | | 3.0 | 3.6 | | 1.4 | | 13 | 38 |
| | (2.3) | (0.40) | (2.0) | (2.4) | (4.5) | BQL | (2.7) | (1.2) | BQL | (1.2) | BQL | (2.4) | (2.6) |

Female

| | | | | | | | | | | | | | |
|---|---|---|---|---|---|---|---|---|---|---|---|---|---|
| 2 | 2.0^{d} | | 14 | 2.4^{e} | 5.4^{d} | 14^{d} | 5.2 | 6.1 | 5.2^{e} | 2.6 | 4.0^{d} | 20 | 20 |
| | (0.77) | BQL | (2.7) | (0.48) | (0.50) | (2.2) | (0.90) | (1.1) | (1.8) | (2.4) | (0.77) | (6.5) | (5.7) |
| 8 | 1.9^{d} | 1.5^{e} | 18^{e} | 2.7 | 5.6 | 10^{d} | 5.6 | 4.9 | 3.3 | 1.6 | 4.9^{d} | 15 | 25 |
| | (0.55) | (1.5) | (0.83) | (0.84) | (4.9) | (0.10) | (0.70) | (0.10) | (1.9) | (1.7) | (1.6) | (2.4) | (6.2) |

^{a}Includes over ten other unidentified minor metabolites
^{b}Standard deviation (n = 3)
^{c}BQL, Below quantitation limit (2 ng equivalents/mL for plasma)
^{d}Significantly different from male, p < 0.01
^{e}Significantly different from male, p < 0.05

TABLE 14
Estimated Plasma CONCENTRATIONS (Ng Equivalents/MI) Of DCDQ And Metabolites In Rats Following
Oral Administration Of [¹⁴C]DCDQ (5 Mg/Kg)^{a}

| | | | | | | | | | | | | |
|---|---|---|---|---|---|---|---|---|---|---|---|---|
| Time (hr) | M1 | M2 | M3 + M9 | M4 | M7 | M8 | M10 | M11 | M12 | M13 | M14 | DCDQ |

Male

| | | | | | | | | | | | | |
|---|---|---|---|---|---|---|---|---|---|---|---|---|
| 2 | 88.4 | 32.3 | 76.4 | 28.3 | 63.6 | 15.1 | 23.7 | 37.2 | | 11.6 | | 86.9 |
| | (1.77)^{b} | (12.1) | (31.6) | (9.26) | (6.12) | (9.12) | (10.5) | (10.5) | BQL^{c} | (3.66) | BQL | (35.2) |
| 4 | 59.7 | 22.6 | 105 | 28.3 | 62.1 | 30.3 | 9.81 | 34.5 | | 14.2 | | 90.8 |
| | (4.88) | (5.01) | (18.6) | (3.39) | (16.7) | (4.81) | (16.7) | (6.82) | BQL | (2.02) | BQL) | (22.8) |
| 8 | 41.9 | 20.0 | 64.0 | 12.4 | 53.0 | | 13.2 | 16.3 | | 6.33 | | 58.7 |
| | (13.7) | (2.96) | (3.77) | (11.4) | (20.6) | BQL | (11.7) | (3.75) | BQL | (5.50) | BQL | (15.5) |

Female

| | | | | | | | | | | | | |
|---|---|---|---|---|---|---|---|---|---|---|---|---|
| 2 | 12.6^{d} | | 92.5 | 15.9 | 35.6^{e} | 91.1^{e} | 35.2 | 41.0 | 33.8^{e} | 16.6 | 25.1^{d} | 132 |
| | (2.14) | BQL | (28.6) | (7.65) | (12.2) | (37.5) | (16.2) | (17.0) | (12.6) | (15.4) | (2.29) | (61.2) |
| 8 | 6.52^{d} | 4.40^{d} | 61.8 | 8.96 | 17.5 | 34.4^{d} | 18.9 | 16.6 | 11.8 | 5.62 | 16.5^{e} | 49.4 |
| | (2.16) | (4.15) | (8.84) | (1.52) | (15.3) | (6.44) | (3.69) | (2.95) | (7.79) | (6.75) | (6.51) | (12.6) |

^{a}Concentrations were estimated based on the total plasma radioactivity concentrations (Table 14) and the percent distribution of plasma radioactivity (Table 13).
^{b}Standard deviation (n = 3)
^{c}BQL, Below quantitation limit (2 ng equivalents/mL for plasma)
^{d}Significantly different from male, p < 0.01
^{e}Significantly different from male, p < 0.05

### Urinary Excretion and Metabolite Profiles

Urine was a major route of excretion, with 66.7±5.0% of the radioactive dose recovered in urine samples in the first 24 hours post-dose, with 32.5% in the 0-8 hour period and 34.2% in the 8-24 hour period. Most of the major plasma metabolites were also detected in urine (Table 15). The major metabolites in urine from male rats included hydroxy DCDQ metabolites (M1, M2, M3 and M4), keto DCDQ (M7) and hydroxy DCDQ glucuronide (M9) (Table 15). The individual metabolites in the 0-24 hour urine represented about 2 to 16% of the administered dose (Table 16), while DCDQ represented less than 1% of the dose. The distribution of metabolites was similar for the 0-8 hour and 8-24 hour collections.

TABLE 15
Chromatographic Distribution (%) Of Radioactivity in male Rat Urine Following
Oral Administration of [¹⁴C]DCDQ (5 mg/kg)

| Time | | | | | | | | | |
|---|---|---|---|---|---|---|---|---|---|
| (hr) | M1 | M2 | M3 + M9 | M4 | M7 | M8 | M10 | DCDQ | Others^{a} |
| 0-8 | 26.9 | 9.51 | 18.8 | 3.73 | 9.68 | 3.56 | 3.32 | 1.29 | 23.2 |
| | (3.43)^{b} | (3.66) | (0.95) | (1.15) | (1.06) | (0.32) | (0.18) | (0.12) | (1.28) |
| 8-24 | 22.5 | 5.83 | 18.8 | 3.08 | 10.1 | 4.36 | 2.70 | 1.46 | 31.2 |
| | (2.96) | (3.09) | (2.13) | (1.05) | (0.22) | (0.60) | (0.52) | (0.63) | (0.85) |

^{a}At least ten additional minor metabolites
^{b}Standard deviation (n = 3)

TABLE 16
Percentage of dose for urinary metabolites in Rats Following Oral
Administration of [¹⁴C]DCDQ (5 mg/kg)

| Time (hr) | M1 | M2 | M3 + M9 | M4 | M7 | M8 | M10 | DCDQ |
|---|---|---|---|---|---|---|---|---|
| 0-8 | 8.67 | 3.16 | 6.13 | 1.21 | 3.16 | 1.15 | 1.08 | 0.42 |
| | (0.64)^{a} | (1.53) | (0.91) | (0.37) | (0.57) | (0.00) | (0.14) | (0.02) |
| 8-24 | 7.74 | 1.94 | 6.45 | 1.03 | 3.44 | 1.47 | 0.91 | 0.51 |
| | (1.87) | (0.85) | (1.45) | (0.29) | (0.45) | (0.13) | (0.07) | (0.28) |
| 0-24 | 16.4 | 5.09 | 12.6 | 2.24 | 6.60 | 2.62 | 1.99 | 0.93 |
| | (2.45) | (2.35) | (1.83) | (0.27) | (0.31) | (0.13) | (0.15) | (0.29) |

^{a}Standard deviation (n = 3)

### Fecal Excretion and Metabolite Profiles

Fecal elimination accounted for 21.1±2.1% of the dosed radioactivity recovered in the first 24 hours post-dosing for male rats. Extraction efficiency for the 8-24 hour fecal samples was 64.3%, while an average of 89.5% of the radioactivity was extracted from incubations of [¹⁴C]DCDQ in control fecal homogenate. Hydroxy DCDQ metabolites (M3 and M4), the hydroxy DCDQ sulfate (M8), the hydroxy DCDQ glucuronide (M9) and the acetylated hydroxy DCDQ (M11) were the major metabolites in the 8-24 hour fecal extracts, with only trace amounts of parent drug detected. Metabolite profiles were not obtained from the 0-8 hour fecal samples because of low radioactivity (less than 0.1% of dosed radioactivity). Incubation of [¹⁴C]DCDQ in fecal homogenate at 37° C. for 24 hours showed no detectable degradation.

Radioactivity Content and Metabolite Profiles in Brain

An average of 84.5% of the radioactivity in brain tissue was extracted. Brain radioactivity concentrations were higher than plasma through 8 hours post-dose, and DCDQ was the predominant drug-related component in rat brain. DCDQ accounted for an average of greater than 90% of the radioactivity in brain extracts for male rats at 2, 4 and 8 hour post-dose, and greater than 94% at 2 and 8 hour post-dose for female rats (Table 17). The average radioactivity concentrations in male and female rat brain were similar and only decreased slightly from 2 hour (5.1 and 6.4 μg equivalents/g for male and female, respectively) to 8 hour (3.2 and 2.8 μg equivalents/g for male and female rats, respectively). By 24 hour, the brain concentration, at an average of 0.04 μg equivalents/g for male rats, was lower than in plasma. The average brain-to-plasma radioactivity ratios between 2 and 8 hour post-dosing were 6.9 to 8.2 for male rats and 8.7 to 9.6 for female rats, and decreased to 0.8 at 24 hour for male rats. There were no significant differences in brain radioactivity content or brain-to-plasma radioactivity ratios between male and female rats. The brain-to-plasma DCDQ ratios were much higher than the radioactivity ratios (Table 17). The average brain-to-plasma DCDQ ratio was between 49.9 and 56.1 independent of time or sex. Only minor amounts of metabolites M7, M10 and M11 were detected in male and female rat brain, and each metabolite represented an average of less than 4.5% of brain radioactivity. Two additional minor metabolites (M1 and M3) were observed in male rat brain. By 8 hour post-dose, most metabolites were not detectable and only DCDQ was observed.

TABLE 17
Brain Concentrations Of Radioactivity And DCDQ Relative To Plasma Concentrations
Following Oral Administration Of [¹⁴C]DCDQ (5 Mg/Kg) To Rats^{a}

| | Concentration | | | | |
|---|---|---|---|---|---|
| | of Radioactivity | | | | |
| | (μg equivalents/g) | | % DCDQ of Brain | Brain/Plasma Ratio | |
| Time (hr) | Total | DCDQ^{b} | Radioactivity | Total | DCDQ |

Male

| | | | | | |
|---|---|---|---|---|---|
| 2 | 5.12 ± 1.28 | 4.63 ± 1.18 | 90.4 ± 0.9 | 8.20 ± 1.95 | 56.4 ± 11.5 |
| 4 | 4.94 ± 0.44 | 4.47 ± 0.42 | 90.4 ± 0.5 | 7.49 ± 0.62 | 50.5 ± 8.88 |
| 8 | 3.25 ± 0.99 | 2.93 ± 0.93 | 90.0 ± 1.2 | 6.92 ± 1.62 | 50.5 ± 11.4 |
| 24 | 0.04 ± 0.00 | NA^{c} | NA | 0.80 ± 0.12 | NA |

Female

| | | | | | |
|---|---|---|---|---|---|
| 2 | 6.38 ± 2.22 | 6.08 ± 2.17 | 95.2 ± 1.0 | 9.57 ± 0.68 | 49.9 ± 14.3 |
| 8 | 2.85 ± 0.68 | 2.68 ± 0.66 | 94.1 ± 0.7 | 8.69 ± 2.86 | 56.1 ± 17.8 |

^{a}Data are presented as mean ± S.D., N = 3
^{b}DCDQ concentrations were estimated on the total radioactivity concentrations in brain and percent distribution of brain radioactivity.
^{c}Not available, concentration below level for profiling

### Metabolite Characterization by LC/MS Analyses

Mass spectra were obtained by LC/MS and LC/MS/MS analysis for DCDQ and its metabolites. Structural characterization of these compounds is summarized in Table 18. The mass spectral characterization of DCDQ and its metabolites are discussed below with the characterization from the other studies described herein.

TABLE 18
Metabolites Of Dcdq Identified In Rats By LC/MS Analysis

| | Retention | | Site(s) of | Metabolite | |
|---|---|---|---|---|---|
| Metabolite | Time (min)^{a} | [M + H]⁺ | Metabolism | Name | Matrix^{b} |
| M1 | 24.9 | 245 | Diazepane ring | Hydroxy DCDQ | P, U, B |
| M2 | 28.8 | 245 | Pyridine ring | Hydroxy DCDQ | P, U |
| M3 | 35.3 | 245 | Cyclopentane | Hydroxy DCDQ | P, U, B, F |
| | | | ring | | |
| M4 | 37.1 | 245 | Cyclopentane | Hydroxy DCDQ | U, F |
| | | | ring | | |
| M7 | 32.2 | 243 | Cyclopentane or | Keto DCDQ | P, U, B |
| | | | pyridine ring | | |
| M8 | 22.0 | 325 | Pyridine ring | Hydroxy DCDQ | P, U, F |
| | | | | Sulfate | |
| M9 | 35.4 | 421 | Cyclopentane | Hydroxy DCDQ | P, U, F |
| | | | ring | Glucuronide | |
| M10 | 41.7 | 245 | Diazepane ring | Hydroxy DCDQ | U, B, F |
| M11 | 48.7 | 287 | Diazepane ring | Acetylated hydroxy | P, B, F |
| | | | | DCDQ | |
| M12 | 60.4 | 309 | Diazepane ring | DCDQ Sulfamate | P, U |
| M13 | 35.6 | 325 | Pyridine ring | Hydroxy DCDQ | |
| | | | | Sulfate | |
| M14 | 15.8 | 305 | Pyridine and | Di-dehydro DCDQ | P, U |
| | | | diazepane ring | Sulfamate | |
| DCDQ | 53.8 | 229 | | | P, U, F |

^{a}LC/MS retention times were normalized to LC/MS data file GU_071803_0003, and GU_081303_0002.
^{b}P = plasma; U = urine; B = brain; F = feces. Fecal metabolites were detected by selective reaction monitoring.

### Discussion

DCDQ was extensively metabolized in rats following a single oral 5 mg/kg administration and oxidative metabolism was the major metabolic pathway. DCDQ represented an average of 13% to 20% of plasma radioactivity between 2 and 8 hour post-dose and less than 2% of total urinary radioactivity at 0-8 and 8-24 hour post-dose. Metabolites observed in plasma included hydroxy DCDQ metabolites (M1, M2, M3, M4 and M10), keto DCDQ (M7), and phase II metabolites such as DCDQ sulfamate (M12), di-dehydro DCDQ sulfamate (M14), hydroxy DCDQ sulfates (M8 and M13), hydroxy DCDQ glucuronide (M9) and acetylated hydroxy DCDQ (M11) (FIG. 1). Percent distribution of radioactivity in plasma did not change significantly over time, except for metabolite M8, which was significantly lower at 8 hour than 2 and 4 hour post-dose. Plasma metabolite profiles exhibited differences in male and female rats. While the hydroxy DCDQ metabolites (M1, M2 and M3), the keto DCDQ (M7) and the hydroxy DCDQ glucuronide (M9) were the major metabolites in male rat plasma, the hydroxy DCDQ metabolite (M3), the hydroxy DCDQ sulfate (M8), the hydroxy DCDQ glucuronide (M9) and DCDQ sulfamate (M12) were the major metabolites in female rats. The primary sex difference was in the formation of sulfates or sulfamates. These sex differences were predictable since sulfotransferase activity toward alcohol and alicyclic amines has been reported to be markedly higher in female rats than in male rats. (Naritomi Y, Niwa T, Shiraga T, Iwasaki K, Noda K. Isolation and characterization of an alicyclic amine N-sulfotransferase from female rat liver. Biological & Pharmaceutical Bulletin. 1994;7:1008-1011.)

Most of the major plasma metabolites were also detected in urine. Similar profiles were obtained for the 0-8 hour and the 8-24 hour urine samples. The major metabolites in urine from male rats included hydroxy DCDQ metabolites (M1, M2, M3 and M4), keto DCDQ (M7) and hydroxy DCDQ glucuronide (M9). Each individual metabolite in the 0-24 hour urine represented about 2 to 16% of the administered dose, while DCDQ represented less than 1% of the dose. In the 8-24 hour fecal samples, the hydroxy DCDQ metabolites (M3 and M4), the hydroxy DCDQ sulfate (M8), the hydroxy DCDQ glucuronide (M9) and the acetylated hydroxy DCDQ (M11) were the major metabolites observed, with only trace amounts of parent drug detected.

Radioactivity in brain tissue was significantly higher than in plasma at 2, 4 and 8 hour post-dose. DCDQ accounted for an average of greater than 90% of brain radioactivity for male and female rats. The average brain-to-plasma radioactivity ratios between 2 and 8 hour post-dose ranged from 6.9 to 9.6, indicating uptake by brain tissue. By 24 hour post-dose, the average brain-to-plasma radioactivity ratio decreased to 0.8. There were no significant differences in brain radioactivity content or brain-to-plasma radioactivity ratios between male and female rats. The average brain-to-plasma DCDQ ratios ranged from 49.9 to 56.1, with no sex differences or changes over time between 2 and 8 hour post-dose. Minor amounts of metabolites M7, M10 and M11 were detected in male and female rat brain. These data indicated that DCDQ readily crossed the blood brain barrier, while uptake of metabolites into brain tissue was limited. The brain-to-plasma radioactivity ratios also suggested that clearance from brain occurred rapidly after 8 hour post-dose, since the ratios decreased from 6.9 to 0.8 by 24 hour post-dose. While partitioning of radioactivity into brain was apparent, partitioning into blood cells was limited with blood-to-plasma ratios of only about 1.1 between 2 and 8 hour post-dose.

Metabolism of DCDQ appeared more extensive in the present study compared with a previous in vitro metabolism study with rat liver microsomes. Only three oxidative metabolites (M2, M3 and M4) were observed with rat liver microsomes and sex differences were not observed. However, sex differences in formation of sulfates and sulfamates, which were observed in rat, were not investigated in any in vitro system. In addition to the metabolites M2, M3 and M4 detected with rat liver microsomes, other oxidative metabolites (M1, M7 and M10) and several phase 11 metabolites (M8, M11, M12, M13 and M14) were also observed in rats (FIG. 1).

In summary, DCDQ was extensively metabolized in rats to predominantly oxidative metabolites. Plasma profiles for male and female rats differed in sulfate and sulfamate conjugates of DCDQ and some oxidative metabolites. DCDQ was the predominant drug related component in brain while only minor amounts of metabolites were observed, and sex differences were not apparent. DCDQ readily crossed the blood brain barrier while uptake of metabolites was limited to minor amounts of oxidative metabolites.

### In Vivo Metabolism of [¹⁴C]DCDQ in Male Dogs Following a Single 15 mg/kg Oral Capsule Administration

Synopsis

The present study investigated metabolism of [¹⁴C]DCDQ in four male beagle dogs following a single administration of 14.1 to 16.7 mg/kg of [¹⁴C]DCDQ hydrochloride in an enteric coated capsule. Plasma samples were collected at 2, 4, 8, 24 and 48 hour post-dose. Feces and urine were collected at intervals of 0-8, 8-24 and 24-48 hour post-dose. Samples were analyzed for radioactivity content and metabolite profiles.

Plasma concentrations of radioactivity were 422±573, 564±748, 528±566, 1340±508 and 507±135 ng equivalents/mL at 2, 4, 8, 24 and 48 hour post-dose, respectively. Large individual variations were observed in plasma radioactivity concentrations, ranging from 4 to 1640 ng equivalents/mL at 2, 4 and 8 hour post-dose. The highest plasma radioactivity concentrations occurred at 24 hour except dog 2, where concentrations were the highest at 4 hour post-dose. The data are consistent with variations in excretion of radioactivity observed in the first 24 hours post-dose. The variability may be associated with slow and prolonged absorption of DCDQ in some dogs, and the enteric-coated capsules. The average blood-to-plasma radioactivity ratio for dog was approximately 0.72.

DCDQ was extensively metabolized in dogs. Oxidative metabolism was the major metabolic pathway, while formation of a DCDQ carbamoyl glucuronide was also observed. DCDQ represented 1.9% to 21% of plasma radioactivity at 2 and 4 hour, less than 3% at 8 and 24 hour, and was not detected at 48 hour post-dose. DCDQ accounted for an average of less than 11% of urinary radioactivity at all time periods. In fecal extracts, 54% to 97% of the radioactivity was attributed to the parent drug. The major metabolites observed in the 2 and 4 hour plasma included hydroxy DCDQ (M1, M2 and M3), an N-oxide DCDQ (M5), a keto DCDQ (M7), a hydroxy DCDQ imine (M15), a hydroxy DCDQ glucuronide (M9) and the carbomoyl glucuronide of DCDQ (M6) (FIG. 1). Metabolites M3 and M9 accounted for the majority of plasma radioactivity at 8, 24 and 48 hour post-dose. Metabolites M2, M3, M5 and M6 were also observed in the in vitro incubation of DCDQ with dog liver microsomes in the presence of NADPH. Metabolites observed in dog plasma were also detected in dog urine except for the metabolite M7. A sulfate conjugate of hydroxy DCDQ (M16) and a diazepinyl DCDQ carboxylic acid (M17), which were not detected in plasma, were observed in urine samples. Hydroxy DCDQ metabolites (M2, M3 and M19), a keto DCDQ (M18) and the hydroxy DCDQ imine (M15) were detected in fecal extracts. Extensive metabolism and prolonged oral absorption of DCDQ probably accounted for the relatively low oral bioavailability of approximately 25.4% in dogs.

Metabolism of DCDQ in dog exhibited some differences from rats. Some different oxidative metabolites were observed in rats and dogs. Oxidative metabolites M15, M16, M17, M18 and M19 were not observed in rats, while a hydroxy metabolite M4, which was observed in rats, was not detected in dogs. More phase II metabolites were observed in rats than in dogs. The sulfates M8 and M13, and sulfamates M12 and M14 were observed in rats, but not in dogs. The sulfate M16 was observed in dogs, but not in rats. The carbamoyl glucuronide of DCDQ, which was detected in dog plasma and urine, was not observed in rat plasma or urine.

In summary, DCDQ was extensively metabolized in dogs, with the oxidative metabolism as the major metabolic pathway, although formation of a DCDQ carbamoyl glucuronide was also observed.

### Introduction

Mass balance studies showed that an average of 64.3% of the oral dose was excreted in rat urine, while 32.7% of the dose was recovered in dog urine following administration of an enteric-coated capsule. When incubated with dog liver microsomes in the presence of NADPH and UDPGA, [¹⁴C]DCDQ was converted to several oxidative metabolites and a carbamoyl glucuronide. A previous metabolism study rats showed that DCDQ was extensively metabolized and oxidative metabolism was the major metabolic pathway in rats. The present study investigated metabolism of [¹⁴C]DCDQ following a single oral capsule administration to dogs.

### Materials and Methods

### Materials

[¹⁴C]DCDQ hydrochloride was synthesized by the radiosynthesis group of Wyeth Research (Pearl River, N.Y.) as described above in the in vivo studies. Ultima Gold, Ultima Flo, Permafluor E+-scintillation cocktails, and Carbo-Sorb E carbon dioxide absorber were purchased from Perkin Elmer (Wellesley, Mass.). EDTA was obtained from Sigma-Aldrich (Milwaukee, Wis.). Solvents used for extraction and for chromatographic analysis were HPLC or ACS reagent grade from EMD Chemicals (Gibbstown, N.J.).

### Methods

### Capsule Preparation and Analysis

About 11 mg of [¹⁴C]DCDQ hydrochloride and 940 mg of non-labeled DCDQ hydrochloride were dissolved in methanol and then evaporated under a nitrogen stream to dryness. Capsules (#2) were filled with accurate amounts (126.7-138.1 mg) of the mixed drug substance according to animal weights. The filled gelatin capsules were then enteric-coated manually.

The drug substance in an extra capsule was analyzed for radiochemical purity and specific activity. An aliquot of the drug substance was dissolved in DMSO, diluted in water, and analyzed by HPLC with radioactivity flow detection and UV detection at 250 nm. To determine the specific activity, non-labeled DCDQ solutions at five different concentrations were prepared by diluting a stock solution in methanol, and analyzed by HPLC to generate a standard curve. The UV peak of [¹⁴C]DCDQ was integrated to calculate the amount of DCDQ against the standard curve. Fractions around the [¹⁴C]DCDQ peak were collected at 1 minute intervals after UV detection. Radioactivity in each fraction was determined by liquid scintillation counting (LSC). Fractions were also collected from a blank injection to obtain the background level of radioactivity.

### Drug Administration and Specimen Collection

Four male beagle dogs, weighing from 7.6 to 9.8 kg at the time of dosing, were from an in-house colony. Each dog was given one enteric-coated capsule containing [¹⁴C]DCDQ as the hydrochloride salt. Animals were fed two hours prior to dosing and provided Purina dog chow and water ad libitum, and were housed individually in metabolic cages.

Blood samples were collected from the jugular vein at 2, 4, 8, 24 and 48 hour after dose administration into tubes containing potassium EDTA as the anticoagulant and then placed on ice. Aliquots of 50 μL were removed for combustion and determination of radioactivity content. Plasma was immediately obtained from the remaining blood by centrifugation at 4° C. Urine samples were collected into tubes on dry ice at intervals of 0-8, 8-24 and 24-48 hour post-dose. Fecal samples were collected at intervals of 0-8, 8-24 and 24-48 hour post-dose at room temperature, and were homogenized. The biological specimens were stored at approximately −70° C. until analysis.

### Radioactivity Determinations

Aliquots of 50 μL of plasma and 100-200 μL of urine were analyzed for radioactivity concentrations. Radioactivity determinations of dose, plasma, and urine were made with a Tri-Carb Model 3100 TR/LL LSC using 5-10 mL of Ultima Gold as the scintillation fluid.

Feces were weighed and homogenized in water at a volume-to-weight ratio of about 5:1. Aliquots of blood (200 μL) and fecal homogenates (0.25-0.53 gram) were placed on Combusto-cones with Combusto-pads and combusted. A model 307 Tri-Carb sample oxidizer, equipped with an Oximate-80 robotic automatic sampler (Perkin Elmer), was used for combustion of blood and fecal samples. The liberated ¹⁴CO₂ was trapped with Carbo-Sorb E carbon dioxide absorber, mixed with PermaFluor® E+ liquid scintillation cocktail, and counted on a Tri-Carb Model 3100 TR/LL liquid scintillation counter (Perkin Elmer). The efficiency of combustion was 98.9%.

For plasma profiles, a TopCount NXT radiometric microplate reader (Perkin Elmer) was used to analyze the radioactivity in collected HPLC fractions. The limit of detection by TopCount was about 1 ng equivalent/mL. A Flo-One β Model A525 radioactivity detector (Perkin Elmer) with a 250 μL LQTR flow cell was used to acquire data for urine and fecal samples. The flow rate of Ultima Flow M scintillation fluid was 1 mL/min, providing a mixing ratio of scintillation cocktail to mobile phase of 5:1. The limits of detection by Flo-One detector were about 200 ng equivalents/mL for urine and 12 ng equivalents/g for feces.

### Plasma Metabolite Profiles

Plasma samples were analyzed for metabolite profiles by HPLC. Aliquots of plasma were mixed with two volumes of cold methanol containing 0.1% trifluoroacetic acid (TFA), placed on ice for about 2 minutes and then centrifuged. The supernatant fluid was transferred to a clean tube and evaporated at 22° C. under nitrogen in a Zymark TurboVap LV (Caliper Life Sciences, Hopkinton, Mass.) to a volume of about 0.3 mL. The residue was centrifuged, the supernatant volume measured and extraction efficiency determined by analysis of duplicate 20 μL aliquots for radioactivity. A 200 μL aliquot of the supernatant was injected onto the HPLC column and the effluent was collected at 20 second intervals into 96-well Lumaplates (Perkin Elmer). The plates were dried overnight in an oven at 40° C. and analyzed by a TopCount. Plasma extracts were also analyzed by LC/MS.

### Analysis of Feces and Urine

Fecal homogenates were analyzed for metabolite profiles. Aliquots of 1 gram of fecal homogenate were mixed with 2 mL methanol, placed on ice for about 10 minutes and centrifuged. The supernatant was transferred to a clean tube. The residue was extracted three times with 2 mL of a water:methanol (3:7) mixture. The supernatants from each sample were combined, evaporated to about 1 mL, and centrifuged. Extraction efficiency was determined by analyzing aliquots of 10 μL of the supernatant for radioactivity. An aliquot (50-200 μL) of the supernatant was analyzed by HPLC with radioactivity flow detection for metabolite profiles. Samples were also analyzed by LC/MS to characterize the radioactive peaks.

Urine was analyzed for radioactivity concentration and analyzed by HPLC with radioactivity flow detection for metabolite profiles by direct injection to the HPLC column. LC/MS analyses for metabolite identification were also carried out with urine samples.

### High Performance Liquid Chromatography

A Waters model 2690 HPLC system (Waters Corp., Milford, Mass.) with a built-in autosampler was used for analysis. Separations were accomplished on a Phenomenex Luna C₁₈(2) column (150×2.0 mm, 5 μm) (Phenomenex, Torrance, Calif.). The sample chamber of the autosampler was maintained at 4° C., while the column was at ambient temperature of about 20° C. A variable wavelength UV detector set to monitor 250 nm and a Flo-One β Model A525 radioactivity detector were used for data acquisition. The HPLC mobile phase consisted of 10 mM ammonium acetate, pH 4.5 (A) and acetonitrile (B), and was delivered at 0.2 mL/min. Chromatographic condition A was used for dose analysis, while condition B was used for analysis of urine and plasma, brain and fecal extracts.

TABLE 19

| | | |
|---|---|---|
| Time (min) | Mobile Phase A (%) | Mobile Phase B (%) |

CONDITION A

| | | |
|---|---|---|
| 0 | 90 | 10 |
| 3 | 90 | 10 |
| 25 | 60 | 40 |
| 45 | 15 | 85 |
| 50 | 15 | 85 |

CONDITION B

| | | |
|---|---|---|
| 0 | 90 | 10 |
| 6 | 90 | 10 |
| 35 | 60 | 40 |
| 65 | 15 | 85 |
| 70 | 15 | 85 |

### LC/MS Analysis

An Agilent Model 1100 HPLC system (Agilent Technologies, Palo Alto, Calif.) including an autosampler and diode array UV detector was used for LC/MS analysis. The UV detector was set to monitor 200 to 400 nm. For selected LC/MS analysis, radiochromatograms were acquired using a β-Ram model 3 radioactivity flow detector (IN/US Systems Inc., Tampa, Fla.) equipped with a solid scintillant flow cell. LC conditions were the same as the condition B described above.

The mass spectrometer used for metabolite characterization was a Micromass Q-TOF-2 quadrupole time-of-flight hybrid mass spectrometer (Waters). The mass spectrometer was equipped with an electrospray ionization (ESI) interface and operated in the positive ionization mode. Collision energy settings of 5 and 30 eV were used for full MS and MS/MS scans, respectively. Settings for the mass spectrometer are listed below.

TABLE 20
Micromass Q-TOF-2 Mass Spectrometer Settings

| Capillary Voltage | 3.0 kV |
|---|---|
| Cone | 30 V |
| Source Block Temperature | 100° C. |
| Desolvation Gas Temperature | 250° C. |
| Desolvation Gas Flow | 550 L/hr |
| Cone Gas Flow | 50 L/hr |
| CID Gas Inlet Pressure | 13-14 psig |
| TOF-MS resolution (m/Δm) | 8000 |

### Data Analysis and Statistical Evaluation

Flo-One analytical software (Perkin Elmer, version 3.6) was utilized to integrate the radioactive peaks. DataFlo Software Utility (Perkin Elmer, beta version 0.55) was used to convert ASCII files from the TopCount NXT microplate counter into CR format for processing in Flo-One Analysis software. The computer program Microsoft Excel® 97 was used to calculate means and standard deviations and to perform the student t-test. Micromass MassLynx software (Waters, version 4.0) was used for collection and analysis of LC/MS data.

### Results

### Analysis of Capsule Content

The [¹⁴C]DCDQ loaded in capsules had an average radiochemical purity of about 98.9% and a chemical purity (by ultraviolet detection) of greater than 99%. The specific activity of [¹⁴C]DCDQ in the capsules was 2.18 μCi/mg as the hydrochloride salt. The actual DCDQ dose administered ranged from 12.2 to 14.4 mg/kg as the free base. Plasma Radioactivity Concentrations and Metabolite Profiles

The concentrations of radioactivity in blood and plasma after a single capsule dose of [¹⁴C]DCDQ are summarized in Table 21.

TABLE 21
Blood And Plasma Concentrations (Ng Equivalents/MI) Of Radioactivity In Dogs
Following A Single 15 Mg/Kg Oral Dose Of [¹⁴C]DCDQ In An Enteric-Coated Capsule

| | Blood | | | | | Plasma | | | | |
|---|---|---|---|---|---|---|---|---|---|---|
| Time (hr) | 1^{a} | 2 | 3 | 4 | Mean ± SD | 1 | 2 | 3 | 4 | Mean ± SD |
| 2 | 161 | 840 | 48.6 | 0.00 | 262 ± 391 | 360 | 1250 | 74.4 | 4.13 | 422 ± 573 |
| 4 | 319 | 1100 | 66.7 | 0.90 | 372 ± 505 | 500 | 1640 | 112 | 4.13 | 564 ± 748 |
| 8 | 331 | 878 | 176 | 5.41 | 347 ± 378 | 541 | 1310 | 256 | 4.13 | 528 ± 566 |
| 24 | 980 | 782 | 1360 | 556 | 920 ± 341 | 1530 | 1110 | 1940 | 769 | 1340 ± 508 |
| 48 | 333 | 434 | 364 | 241 | 343 ± 79.9 | 504 | 669 | 517 | 339 | 507 ± 135 |

^{a}Dog number

The average plasma radioactivity concentrations ranged from 423 ng equivalents/mL at 2 hour to 1340 ng equivalents/mL at 24 hour post-dose. The highest radioactivity concentration generally occurred at 24 hour post-dose except for dog 2, where concentrations were the highest at 4 hour post-dose. Large individual variations were observed in plasma radioactivity concentrations, ranging from 4 to 1640 ng equivalents/mL at 2, 4 and 8 hour post-dose. The data are in agreement with the large variations in excretion of radioactivity observed in the first 24 hours post-dose. These variations may be attributed to slow and prolonged absorption of DCDQ in some dogs. Blood radioactivity concentrations were lower than plasma radioactivity levels, and the average blood-to-plasma radioactivity ratios ranged between 0.68 and 0.79 (Table 22).

Partitioning of DCDQ and its metabolites into blood cells was limited based on these ratios.

TABLE 22
Blood-To-Plasma Radioactivity Ratios In Dogs Following A Single
15 Mg/Kg Oral Dose Of [¹⁴C]DCDQ In An Enteric-Coated Capsule

Blood-to-Plasma Ratios

| Time (hr) | 1^{a} | 2 | 3 | 4 | Mean ± SD |
|---|---|---|---|---|---|
| 2 | 0.51 | 0.77 | 0.75 | NA^{b} | 0.68 ± 0.14 |
| 4 | 0.73 | 0.77 | 0.69 | NA^{b} | 0.73 ± 0.04 |
| 8 | 0.70 | 0.77 | 0.79 | NA^{b} | 0.75 ± 0.05 |
| 24 | 0.73 | 0.81 | 0.80 | 0.83 | 0.79 ± 0.04 |
| 48 | 0.76 | 0.74 | 0.81 | 0.82 | 0.78 ± 0.04 |

^{a}Dog number
^{b}Data not available due to the low radioactivity concentrations.

The extraction recovery was greater than 71% of the plasma radioactivity. DCDQ was extensively metabolized in dogs (Tables 23 and 24). At 2 and 4 hour post-dose DCDQ represented 1.9% to 21% of plasma radioactivity. DCDQ represented less than 3% of plasma radioactivity at 8 and 24 and was not detectable at 48 hour post-dose (Tables 23 and 24). The major metabolites observed in the 2 and 4 hour plasma included hydroxy DCDQ metabolites (M2 and M3), an N-oxide DCDQ (M5), a keto DCDQ (M7), an imine of hydroxy DCDQ (M15), a glucuronide of hydroxy DCDQ (M9) and a carbamoyl glucuronide of DCDQ (M6). Similar profiles were obtained for the 8, 24 and 48 plasma samples, although the majority of radioactivity at these later time points was attributed to the hydroxy metabolite M3 and the glucuronide M9, which were not chromatographically separated. A number of relatively minor metabolites accounted for 6.2 to 42% of plasma radioactivity in the 2 and 4 hour samples. These metabolites were not characterized due to low concentrations.

TABLE 23
Chromatographic Distribution (Percentage) Of Radioactivity In Dog Plasma Following A
Single 15 mg/kg Oral Dose Of [¹⁴C]DCDQ In An Enteric-Coated Capsule^{a}

| Time | Dog | | | | | | | | | |
|---|---|---|---|---|---|---|---|---|---|---|
| (hr) | No. | M1 | M2 | M3 + M9 | M5 | M6 | M7 | M15 | DCDQ | Others^{b} |
| 2 | 1 | 8.4 | ND^{c} | 11 | 12 | ND | ND | 17 | 11 | 41 |
| | 2 | 2.6 | 8.0 | 21 | 2.9 | 19 | ND | 2.8 | 1.9 | 42 |
| 4 | 1 | 8.0 | 6.6 | 28 | 6.6 | ND | 6.9 | 8.5 | 11 | 25 |
| | 2 | 4.9 | 12 | 20 | 5.3 | 13 | 7.8 | 9.6 | 21 | 6.2 |
| 8 | 1 | 8.0 | ND | 43 | 4.0 | 7.0 | ND | 8.3 | 2.7 | 27 |
| | 2 | 4.7 | ND | 79 | ND | ND | ND | 7.3 | ND | 8.8 |
| | 3 | 9.7 | ND | 85 | ND | ND | ND | ND | ND | 5.1 |
| 24 | 1 | 6.8 | ND | 61 | ND | 17 | 13 | ND | 2.6 | 0.0 |
| | 2 | 2.1 | 5.0 | 73 | ND | 5.6 | 6.7 | ND | ND | 7.7 |
| | 3 | 6.4 | 7.0 | 62 | ND | ND | ND | ND | 2.3 | 22 |
| 48 | 1 | 12 | ND | 66 | ND | ND | ND | ND | ND | 22 |
| | 2 | 6.7 | ND | 85 | 2.8 | ND | ND | ND | ND | 5.6 |
| | 3 | ND | ND | 95 | ND | ND | ND | ND | ND | 4.7 |

^{a}Profiles for the 2 and 4 hour samples for dog 3 and 4, and the 8, 24 and 48 hour samples for dog 4 were not obtained.
^{b}Includes non-characterized metabolites
^{c}Not detected

TABLE 24
Estimated Plasma Concentrations (ng equivalents/mL) Of
DCDQAnd Metabolites In Dogs Following A Single 15 Mg/Kg
Oral Dose Of [¹⁴C]DCDQ In An Enteric-Coated Capsule^{a}

| Time (hr) | Dog No. | M1 | M2 | M3 + M9 | M5 | M6 | M7 | M15 | DCDQ |
|---|---|---|---|---|---|---|---|---|---|
| 2 | 1 | 30.1 | BQL^{b} | 37.8 | 43.1 | BQL | BQL | 62.2 | 39.6 |
| | 2 | 32.9 | 99.5 | 264 | 36.4 | 239 | BQL | 35.6 | 23.2 |
| 4 | 1 | 40.0 | 33.1 | 142 | 33.1 | BQL | 34.5 | 42.7 | 52.5 |
| | 2 | 80.9 | 192 | 335 | 86.6 | 215 | 127 | 157 | 345 |
| 8 | 1 | 43.3 | BQL | 234 | 21.7 | 37.9 | BQL | 45.1 | 14.5 |
| | 2 | 61.9 | BQL | 1030 | BQL | BQL | BQL | 95.3 | BQL |
| | 3 | 24.8 | BQL | 218 | BQL | BQL | BQL | BQL | BQL |
| 24 | 1 | 104 | BQL | 929 | BQL | 258 | 202 | BQL | 39.6 |
| | 2 | 23.0 | 55.3 | 808 | BQL | 62.1 | 73.6 | BQL | BQL |
| | 3 | 125 | 135 | 1210 | BQL | BQL | BQL | BQL | 43.8 |
| 48 | 1 | 58.0 | BQL | 333 | BQL | BQL | BQL | BQL | BQL |
| | 2 | 44.7 | BQL | 568 | 18.9 | BQL | BQL | BQL | BQL |
| | 3 | BQL | BQL | 492 | BQL | BQL | BQL | BQL | BQL |

^{a}Data for the 2 and 4 hour samples for dog 3 and 4, and the 8, 24 and 48 hour samples for dog 4 were not obtained due to low concentrations of circulating radioactivity; concentrations were estimated based on the total plasma radioactivity concentrations (Table 21) and the chromatographic distribution of the radioactivity (Table 23).
^{b}Below quantitation limit (1 ng equivalent/mL for plasma).

### Urinary Metabolite Profiles

Urine was a major route of elimination of DCDQ in dog, although fecal excretion was greater than urinary excretion. Numerous metabolites were detected in urine. DCDQ represented an average of less than 11% of the urinary radioactivity for all time points (Table 25). The major metabolites included hydroxy DCbQ metabolites (M2 and M3), an N-oxide DCDQ (M5), an imine of hydroxy DCDQ (M15), a hydroxy DCDQ sulfate (M16), a diazepinyl DCDQ carboxylic acid (M17), a hydroxy DCDQ glucuronide (M9) and a carbamoyl glucuronide of DCDQ (M6) (FIG. 1).

TABLE 25
Chromatographic Distribution (Percentage) Of Radioactivity In Dog Urine
Following Administration Of An Enteric-Coated Capsule Containing [¹⁴C]DCDQ^{a}

| Time | Dog | | | | | | | | | | | |
|---|---|---|---|---|---|---|---|---|---|---|---|---|
| (hr) | No. | M1 | M2 | M3 + M9 | M5 | M6 | M7 | M15 | M16 | M17 | DCDQ | Others^{c} |
| 0-8 | | ND^{b} | 10 | 10 | 5.9 | 13 | ND | 11 | 16 | 11 | ND | 24 |
| 8-24 | 1 | 8.3 | 21 | 11 | ND | 6.1 | ND | 9.2 | 9.3 | 4.1 | 9.5 | 22 |
| | 2 | 4.7 | 26 | 14 | ND | 7.6 | ND | 4.1 | 3.3 | 2.8 | ND | 38 |
| | 3 | 6.7 | 26 | 16 | ND | 3.6 | ND | 2.3 | 3.7 | 3.8 | 11 | 28 |
| | Mean | 6.6 | 24 | 13 | NA^{d} | 5.8 | NA | 5.2 | 5.5 | 3.5 | NA | 29 |
| | SD | 1.8 | 2.8 | 2.3 | NA | 2.0 | NA | 3.6 | 3.3 | 0.7 | NA | 8.4 |
| 24-48 | 1 | 4.4 | 25 | 11 | 8.5 | 5.8 | 5.0 | ND | 8.4 | ND | 3.8 | 28 |
| | 2 | 7.6 | 18 | 14 | 4.0 | 3.5 | 6.2 | ND | 5.0 | 3.4 | ND | 39 |
| | 3 | 7.6 | 22 | 14 | ND | 4.3 | 9.2 | ND | 3.2 | ND | 3.0 | 37 |
| | 4 | 3.2 | 21 | 13 | ND | 5.9 | 3.4 | 3.5 | 5.5 | 5.7 | 6.9 | 32 |
| | Mean | 5.7 | 22 | 13 | NA | 4.7 | 5.9 | NA | 5.5 | NA | 3.4 | 34 |
| | SD | 2.3 | 2.8 | 1.2 | NA | 1.2 | 2.5 | NA | 2.2 | NA | 2.8 | 4.8 |

^{a}The 0-8 hour samples for dogs 2, 3 and 4, and the 0-24 hour sample for dog 4 did not have enough radioactivity for profiling.
^{b}ND, not detected
^{c}It includes a number of non-characterized metabolites
^{d}Not available

### Fecal Metabolite Profiles

An average of 70.2% of the fecal radioactivity was extracted, while an average of 88.2% of the radioactivity was extracted from incubations of [¹⁴C]DCDQ in blank fecal homogenate. In fecal extracts, DCDQ was major radioactive component, representing 54.4% to 96.7% of the total radioactivity. The metabolites detected in feces included hydroxy DCDQ (M2, M3 and M19), a keto DCDQ (M18) and an uncharacterized peak (M20). The most abundant metabolite M18 represented up to 16.4% of the total radioactivity in fecal extracts. The glucuronide of hydroxy DCDQ (M9) and the carbamoyl glucuronide of DCDQ (M6) were not detected in feces. Incubation of [¹⁴C]DCDQ in fecal homogenate at 37° C. for 24 hours showed no obvious degradation (data not shown).

### Metabolite Characterization by LC/MS Analyses

Mass spectra were obtained by LC/MS and LC/MS/MS analysis for DCDQ and its metabolites. Structural characterization of these compounds is summarized in Table 26. The mass spectral characterization of DCDQ and its metabolites, from each of the studies described herein are discussed further below.

TABLE 26
Metabolites Of DCDQ Identified In Dogs By LC/MS Analysis

| | Retention | | Site(s) of | Metabolite | |
|---|---|---|---|---|---|
| Metabolite | Time (min)^{a} | [M + H]⁺ | Metabolism | Name | Matrix^{b} |
| M1 | 26.26 | 245 | Diazepane ring | Hydroxy DCDQ | P, U |
| M2 | 29.46 | 245 | Pyridine ring | Hydroxy DCDQ | P, U, F |
| M3 | 35.95 | 245 | Cyclopentane ring | Hydroxy DCDQ | P, U, F |
| M6 | 59.80 | 449 | Diazepane ring | Carbamate | P, U |
| | | | | glucuronide DCDQ | |
| M7 | 33.74 | 243 | Cyclopentane or | Keto DCDQ | P, U |
| | | | pyridine ring | | |
| M9 | 36.12 | 421 | Cyclopentane ring | Hydroxy DCDQ | P, U |
| M15 | 42.42 | 243 | Cyclopentane or | Hydroxy DCDQ imine | P, U, F |
| | | | pyridine ring | | |
| M16 | 44.26 | 325 | Pyridine and | Hydroxy DCDQ | U |
| | | | diazepane ring | Sulfate | |
| M17 | 48.00 | 257 | Pyridine and | Diazepinyl DCDQ | U |
| | | | diazepane ring | carboxylic acid | |
| M18 | 41.72 | 243 | Diazepane ring | Keto DCDQ | F |
| M19 | 45.24 | 245 | Pyridine or | Hydroxy DCDQ | F |
| | | | Benzene ring | | |
| DCDQ | 53.84 | 229 | | | P, U, F |

^{a}LC/MS retention times were normalized to LC/MS data file GU_072303_0004, GU_072403_0004, and GU_081403_0005
^{b}Matrix where metabolites were detected and characterized by LC/MS, P = plasma;
U = urine; B = brain; F = feces

### Discussion

Large individual variations were observed in plasma radioactivity concentrations, ranging from 4 to 1640 ng equivalents/mL at 2, 4 and 8 hour post-dose. The data are consistent with the variations in excretion of radioactivity observed in the first 24 hours post-dose. Urinary excretion varied from 0 to 25% while fecal excretion ranged from 0 to 23% of the dosed radioactivity in the first 24 hours post-dose. The highest plasma radioactivity concentrations occurred at 24 hour except dog 2, where concentration were the highest at 4 hour post-dose. The variability may be associated with slow and prolonged absorption of DCDQ in some dogs, and possibly the enteric-coated capsules. The average blood-to-plasma radioactivity ratio for dog was approximately 0.72 compared with about 1.1 for rat between 2 and 8 hour post-dose, indicating less uptake of DCDQ and its metabolites into blood cells of dog than of rat.

DCDQ was extensively metabolized in dogs as seen in rats, following administration of an enteric-coated capsule containing [¹⁴C]DCDQ (FIG. 1). Oxidative metabolism was the major metabolic pathway, while formation of a DCDQ carbamoyl glucuronide, which was not observed in rats, was also observed. DCDQ represented 1.9% to 21% of plasma radioactivity at 2 and 4 hour, less than 3% at 8 and 24 hour, and was not detected at 48 hour post-dose. DCDQ accounted for an average of less than 11% of urinary radioactivity at all time points. In fecal extracts, 54.4% to 96.7% of the radioactivity was attributed to the parent drug. Plasma metabolites at 2 and 4 hour post-dose included hydroxy DCDQ (M1, M2 and M3), an N-oxide DCDQ (M5), a keto DCDQ (M7), a hydroxy DCDQ imine (M15), a hydroxy DCDQ glucuronide (M9) and the carbomoyl glucuronide of DCDQ (M6). The majority of radioactivity at 8, 24 and 48 hour post-dose was attributed to the hydroxy metabolite M3 and the glucuronide M9, which were not chromatographically separated. Metabolites M2, M3, M5 and M6 were also observed in the in vitro incubation of DCDQ with dog liver microsomes in the presence of NADPH. Metabolites observed in dog plasma were also detected in dog urine except for the metabolite M7. A sulfate conjugate of hydroxy DCDQ (M16) and a diazepinyl DCDQ carboxylic acid (M17), which were not detected in plasma, were observed in urine samples. Hydroxy DCDQ metabolites (M2, M3 and M19), a keto DCDQ (M18) and a hydroxy DCDQ imine were detected in fecal extracts. Formation of metabolite M6 may be underestimated due to possible hydrolysis in the GI tract. Extensive metabolism and prolonged oral absorption of DCDQ probably accounted for the relatively low oral bioavailability of approximately 25.4% in dogs.

Metabolism of DCDQ in dog exhibited some differences from rats (FIG. 1). Some different oxidative metabolites were observed in rats and dogs. Oxidative metabolites M15, M16, M17, M18 and M19 were not observed in rats, while a hydroxy metabolite M4, which was observed in rats, was not detected in dogs. More phase II metabolites were observed in rats than in dogs. The sulfates M8 and M13, and sulfamates M12 and M14 were observed in rats, but not in dogs. The sulfate M16 was observed in dogs, but not in rats. The carbamoyl glucuronide of DCDQ, which was detected in dog plasma and urine, was not observed in rat plasma or urine.

In summary, DCDQ was extensively metabolized in dogs, with the oxidative metabolism as the major metabolic pathway, although formation of a DCDQ carbamoyl glucuronide was also observed.

### Metabolite Characterization by LC/MS Analyses

Mass spectra were obtained by LC/MS and LC/MS/MS analysis for DCDQ and its metabolites identified in the studies above. The mass spectral characterization of DCDQ and its metabolites, from each of the studies, are discussed below.

### DCDQ

The mass spectral characteristics of DCDQ standard were examined for comparison with the metabolites. In the LC/MS spectrum of DCDQ, a protonated molecular ion, [M+H]⁺, was observed at m/z 229. The product ions of m/z 229 mass spectrum of DCDQ obtained from collision-induced dissociation (CID), and the proposed fragmentation scheme indicated loss of methyleneamine, ethylideneamine, and ethylidene-methyl-amine from the molecular ion generated the product ions at m/z 200, 186, and 171, respectively. Loss of the propene group from the molecular ion generated the fragment at m/z 187 and further loss of methyleneamine and ethylideneamine generated the fragments ions at m/z 158 and 144. Loss of the cyclopentyl-methyleneamine group generated the fragment ion at m/z 132.

### Metabolite M1: from In Vitro, and In Vivo Rat and Dog Studies

The [M+H]⁺ for M1 was observed at m/z 245. The product ions of m/z 245 mass spectrum of M1 and the proposed fragmentation scheme indicated an increase of 16 Da, suggesting monohydroxylation. Loss of propene from the molecular ion generated the fragment at m/z 203, which was 16 Da higher than the corresponding ion at m/z 187 for DCDQ. The fragment ions at m/z 171 and 186 were the same as in the product ion spectrum of DCDQ, indicating that the hydroxylation occurred in the diazepane portion of the molecule as shown. Therefore, M1 was proposed to be hydroxy DCDQ.

### Metabolite M2: from from In Vitro, and In Vivo Rat and Dog Studies

The [M+H]⁺ for M2 was observed at m/z 245. The product ions of m/z 245 mass spectrum of M2 and the proposed fragmentation scheme indicated an increase of 16 Da, suggesting monohydroxylation. Loss of propene from the molecular ion generated the fragment at m/z 203, which was 16 Da higher than the corresponding ion at m/z 187 for DCDQ. This indicated that the cyclopentane ring was not the site of biotransformation. The fragment ion at m/z 132 was the same for DCDQ indicating that the diazepane portion was not the site of biotransformation. The fragment ions at m/z 169 and 184 were 2 Da less than the corresponding ions for DCDQ at m/z 171 and 186, respectively, indicating loss of H₂O from the pyridine ring as a result of hydroxylation. Therefore, M2 was proposed to be hydroxy DCDQ.

### Metabolite M3: from In Vitro, and In Vivo Rat and Dog Studies

The [M+H]⁺ for M3 was observed at m/z 245. The product ions of m/z 245 mass spectrum of M3 and the proposed fragmentation scheme indicated an increase of 16 Da, suggesting monohydroxylation. Loss of ethylideneamine generated the fragment at m/z 202, which was 16 Da higher than the corresponding ion at m/z 186 for SAX-187. The fragment ions at m/z 158 and 144 were the same as in the product ion spectrum of DCDQ. This indicated that the hydroxylation occurred in the cyclopentane portion of the molecule as shown. Therefore, M3 was proposed to be hydroxy DCDQ.

### Metabolite M4: from In Vitro and In Vivo Rat Studies

The [M+H]⁺ for M4 was observed at m/z 245. The product ions of m/z 245 mass spectrum of M4 and the proposed fragmentation scheme indicated an increase of 16 Da, suggesting monohydroxylation. Loss of H₂O from the molecular ion generated the fragment at m/z 227. The product ion at m/z 144 was also observed for DCDQ, indicating that the hydroxylation occurred in the cyclopentane portion of the molecule as shown. This was also consistent with the presence of the m/z 184 product ion, generated by loss of ethylideneamine and H₂O from the corresponding ion at m/z 186 for DCDQ. The measured accurate mass for this ion was 184.1120 Da, which was within 3.6 ppm of the theretical mass for C₁₃H₁₄N. Therefore, M4 was proposed to be hydroxy DCDQ.

### Metabolite M5: from In Vitro Studies

The [M+H]⁺ for M5 was observed at m/z 243. The measured accurate mass for M5 was 243.1478 Da, which was within 7.9 ppm of the theoretical mass for C₁₅H₁₉N₂O. This corresponded to the addition of one oxygen and loss of two hydrogen atoms compared with the molecular formula for DCDQ. A fragment ion at m/z 130 was 2 Da less than the corresponding ion for DCDQ, suggesting the formation of the imine. LC/MS with D₂O substituted for H₂O in the mobile phase confirmed that no exchangeable protons existed for M5, indicating that M5 was an N-oxide. Therefore, M5 was proposed to be the N-oxide of DCDQ imine.

### Metabolite M6: from In Vitro and In Vivo Dog Studies

The [M+H]⁺ for M6 was observed at m/z 449. The product ions of m/z 449 mass spectrum of M6 and the proposed fragmentation scheme indicated a loss of 176 Da from the molecular ion generated a fragment at m/z 273, indicating that M5 was a glucuronide conjugate. Further loss of 44 Da from m/z 273 generated m/z 229, which was also the molecular ion for DCDQ. Therefore, M6 was proposed to be the carbamoyl glucuronide of DCDQ.

### Metabolite M7: from In Vitro and In Vivo Rat and Dog Studies

The [M+H]⁺ for M7 was observed at m/z 243. The product ions of m/z 243 mass spectrum of M7 and the proposed fragmentation scheme indicated loss of methyleneamine, ethylideneamine from the molecular ion generated the product ions at m/z 214 and 200, which were 14 Da more than the corresponding ions at m/z 200 and 186, respectively, for DCDQ. This suggested the addition of one oxygen atom and loss of two hydrogen atoms from DCDQ. The product ions at m/z 132, 144 and 158 were the same as DCDQ, which indicated that the biotransformation occurred in the pyridine and cyclopentane rings. LC/MS with D₂O substituted for H₂O in the mobile phase confirmed that there was only one exchangeable proton for M7, which was from the NH group in the diazepane ring. Therefore, M7 was proposed to be a keto DCDQ.

### Metabolite M8: from In Vivo Rat Studies

The [M+H]⁺ for M8 was observed at m/z 325. The product ions of m/z 325 mass spectrum of M8 and the proposed fragmentation scheme indicated a loss of propene from the molecular ion generated the product ion at m/z 283, indicating the biotransformation did not occur on the cyclopentane ring. Loss of methyleneamine, ethylideneamine from the product ion at m/z 283 and subsequent loss of sulfate group generated the product ions at m/z 158 and 144, respectively. Loss of ethylideneamine from the molecular ion generated the product ion at m/z 282 and subsequent loss of the sulfonate group and H₂O generated the product ions at m/z 202 and 184, respectively. The fragment ion at m/z 132 was the same as for DCDQ indicating that the diazepane portion was not the site of biotransformation. Therefore, M8 was proposed to be sulfate conjugate of hydroxy DCDQ.

### Metabolite M9: from In Vivo Rat and Dog Studies

The [M+H]⁺ for M9 was observed at m/z 421. The product ions of m/z 421 mass spectrum of M9 and the proposed fragmentation scheme indicated a loss of 176 Da from the molecular ion generated the fragment ion at m/z 245, which indicated glucuronidation of hydroxy DCDQ. Loss of ethylideneamine and glucuronic acid generated the fragment at m/z 202, which was 16 Da higher than the corresponding ion at m/z 186 for DCDQ. The fragment ion at m/z 187 suggested that the biotransformation occurred in the cyclopentane ring. Therefore, M9 was proposed to be a glucuronide of hydroxy DCDQ.

### Metabolite M10

The [M+H]⁺ for M10 was observed at m/z 245. The product ions of m/z 245 mass spectrum of M10 and the proposed fragmentation scheme indicated an increase of 16 Da, suggesting monohydroxylation. The fragment ions at m/z 171 and 186 were the same as in the product ion spectrum of DCDQ, indicating that the hydroxylation occurred in the diazepane portion of the molecule as shown. Therefore, M10 was proposed to be hydroxy DCDQ.

### Metabolite M11: from In Vivo Rat Studies

The [M+H]⁺ for M11 was observed at m/z 287. The product ions of m/z 287 mass spectrum of M11 and the proposed fragmentation scheme indicated a loss of H₂O from the molecular ion generated the fragment ion at m/z 269. Further loss of 42 Da generated m/z 227, which indicated acetylation. The fragment ions at m/z 171 and 186 were the same as in the product ion spectrum of DCDQ, indicating that the biotransformations occurred in the diazepane portion of the molecule as shown. Therefore, M11 was proposed to be acetylated hydroxy DCDQ.

### Metabolite M12: from In Vivo Rat Studies

The [M+H]⁺ for M12 was observed at m/z 309. The product ions of m/z 309 mass spectrum of M12 indicated a loss of 80 Da generated the product ion at m/z 229, which is the molecular ion of DCDQ. This indicated sulfation. Further loss of methyleneamine, ethylideneamine generated the product ions at m/z 200 and 186, which were the same for DCDQ. Therefore, M12 was proposed to be the N-sulfate of DCDQ.

### Metabolite M13: from In Vivo Rat Studies

The [M+H]⁺ for M13 was observed at m/z 325. The product ions of m/z 325 mass spectrum of M13 and the proposed fragmentation scheme indicated a loss of 80 Da from [M+H]⁺ yielded m/z 245 which was 16 Da larger than the [M+H]⁺ for DCDQ. This indicated that M13 was a sulfate conjugate of hydroxy DCDQ. Therefore, M13 was proposed to be sulfate conjugate of hydroxy DCDQ.

### Metabolite M14: from In Vivo Rat Studies

The [M+H]⁺ for M14 was observed at m/z 305. The product ions of m/z 305 mass spectrum of M14, the product ions of m/z 225 mass spectrum and the proposed fragmentation scheme for M14 indicated a loss of 80 Da from the molecular ion generated the ion at m/z 225, which indicated that M14 was a sulfate. Further loss of ethylideneamine, and ethylidene-methyl-amine generated the product ions at 182 and 167, respectively, which were 4 Da less than the corresponding ions for DCDQ at m/z 186 and 171, respectively, which indicated metabolism of the cyclopentane group. Therefore, M14 was proposed to be the sulfate conjugate of di-dehydro DCDQ.

### Metabolite M15: from In Vivo Dog Studies

The [M+H]⁺for M15 was observed at m/z 245. The product ions of m/z 245 mass spectrum of M15 and the proposed fragmentation scheme indicated a fragment ion at m/z 187 was 16 Da more than the corresponding ion at m/z 171 for DCDQ, indicating hydroxylation of the cyclopentane or the pyridine ring. The fragment ion at m/z 130 was 2 Da less than the corresponding ion for DCDQ indicating the formation of imine. LC/MS with D₂O substituted for H₂O in the mobile phase confirmed that there was only one exchangeable proton for M15. Therefore, M15 was proposed to be hydroxy DCDQ imine.

### Metabolite M16: from In Vivo Dog Studies

The [M+H]⁺ for M16 was observed at m/z 325. The product ions of m/z 325 mass spectrum of M13 and the proposed fragmentation scheme indicated a loss of 80 Da from the molecular ion generated the product ion at m/z 245, indicating sulfation. Loss of propene from the molecular ion generated the product ion at m/z 283, indicating the biotransformation did not occur on the cyclopentane ring. Loss of ethylideneamine generated the product ion at m/z 282 and subsequent loss of sulfate group and H₂O generated the product ions at m/z 202 and 184 respectively. The product ion at m/z 148, 16 Da more than the corresponding ion at 132 for DCDQ, and the m/z 282 product ion indicated that the hydroxylation occurred in the benzyl group of the molecule as shown. Therefore, M16 was proposed to be sulfate conjugate of hydroxy DCDQ.

### Metabolite M17: from In Vivo Dog Studies

The [M+H]⁺ for M17 was observed at m/z 257. The measured accurate mass of [M+H]⁺ was 257.1292 Da, which was within 0.8 ppm of the theoretical mass for C₁₅H₁₇N₂O₂. This corresponded to the addition of two oxygen atoms and loss of 4 hydrogen atoms compared to the molecular formula of DCDQ. Loss of 44 Da from the molecular ion generated the fragment at m/z 213. The measured accurate mass of this fragment was 213.1376 Da, which was within 7.6 ppm of the theoretical mass for C₁₄H₁₇N₂. This confirmed that the loss of 44 was from the neutral loss of CO₂, indicating that M17 was a carboxylic acid. Further loss of cyclopentene, pentane, propene and HCN from m/z 213 generated the fragments at m/z 145, 171 and 186 respectively. The product ion at m/z 130 was 2 Da less than the corresponding ion at m/z 132 for DCDQ indicating the formation of an imine. LC/MS with D₂O substituted for H₂O in the mobile phase confirmed that there was only one exchangeable proton for M17, which was from the carboxylic acid group. Therefore, M17 was proposed to be Benzo-diazepinyl-cyclopentanecarboxylic acid (diazepinyl DCDQ carboxylic acid).

### Metabolite M18: from In Vivo Dog Studies

The [M+H]⁺ for M18 was observed at m/z 243. The product ions of m/z 243 mass spectrum of M18 and the proposed fragmentation scheme indicated a loss of propene and ethylideneamine groups from the molecular ion generated the product ion at m/z 158. Loss of methyleneamine, ethylideneamine from the molecular ion generated the product ions at m/z 214 and 200, which were 14 Da more than the corresponding ions at m/z 200 and 186, respectively, for DCDQ. This suggested the addition of one oxygen atom and loss of two hydrogen atoms from DCDQ. The product ion at m/z 146, 14 Da more than the corresponding ion at m/z 132 for DCDQ, and the m/z 200 product ion indicated that the modification occurred on the benzyl group. LC/MS with D₂O substituted for H₂O in the mobile phase confirmed that there was only one exchangeable proton for M14, which was from the NH group in the diazepane ring. Therefore, M18 was proposed to be a keto DCDQ.

### Metabolite M19: from In Vivo Dog Studies

The [M+H]⁺ for M19 was observed at m/z 245. The product ions of m/z 245 mass spectrum of M19 and the proposed fragmentation scheme indicated an increase of 16 Da, suggesting monohydroxylation. The product ions at m/z 216, 202 and 187 were 16 Da more than the corresponding ions at m/z 200, 186 and 171, respectively, for DCDQ, indicating that diazepane group was not the site of modification. Loss of propene from the molecular ion generated the fragment at m/z 203 and further loss of methyleneamine and ethylideneamine generated the fragment ions at m/z 174 and 160. These were 16 Da more than the corresponding ions for DCDQ, indicating that hydroxylation occurred at either the benzene or pyridine group. Therefore, M19 was proposed to be hydroxy DCDQ.

### Product P3: from In Vivo Studies

The [M+H]⁺ for P3 was observed at m/z 227. The product ions of m/z 227 mass spectrum of P3 and the proposed fragmentation scheme indicated the molecular weight for P3 was 2 Da less than DCDQ suggesting the formation of a double bond. The fragment ion at m/z 130 was 2 Da less than the corresponding ion for DCDQ, suggesting the formation of an imine. Therefore, P3 was proposed to be DCDQ imine.

### Identification of Metabolites M7, M9 and M13 in Rat Urine Following a Single Oral 50 mg/kg Administration of DCDQ

Synopsis

This study was designed to obtain rat urine for metabolite isolation and to obtain more specific structural identification for selected metabolites of DCDQ. Three male and three female rats were given a single 50 mg/kg dose of DCDQ. Urine was collected at 0-12 and 12-24 hour intervals. DCDQ metabolites M7 (keto DCDQ), M9 (hydroxy DCDQ glucuronide) and M13 (hydroxy DCDQ sulfate) were isolated from the urine by a two stage semi-preparative HPLC method in low microgram quantities sufficient for NMR spectroscopic analysis. Based upon MS and NMR spectroscopic analysis the site of metabolism for M7 and M13 was at 17 position 17. The site of metabolism for M9 was at position 13.

Introduction

When incubated with rat liver microsomes in the presence of NADPH and UDPGA, [¹⁴C]DCDQ was converted to several oxidative metabolites. A previous metabolism study in rats showed that DCDQ was extensively metabolized and oxidative metabolism was the major metabolic pathway in rats. Phase II metabolites including a sulfate and a glucuronide of hydroxy DCDQ were also detected in rats. The present study was designed to obtain rat urine for metabolite isolation and to obtain more specific structural identification for selected metabolites of DCDQ.

### Materials and Methods

### Materials

DCDQ hydrochloride was synthesized by Wyeth Research as described above. Polysorbate 80 was obtained from Mallinckrodt Baker (Phillipsburg, N.J.) and methylcellulose was from Sigma-Aldrich (Milwaukee, Wis.). Solvents used for extraction and for chromatographic analysis were HPLC or ACS reagent grade from EMD Chemicals (Gibbstown, N.J.). Deuterated dimethyl sulfoxide (DMSO-d6) was purchased from Cambridge Isotope Laboratories (Andover, Mass.). NMR tubes (3 mm) were purchased from Wilmad Glass Co. (Buena, N.J.).

### Methods

Drug Administration and Specimen Collection

Dose preparation, animal dosing and specimen collection were performed at Wyeth Research, Collegeville, Pa. The dose vehicle contained 2% (v/v) Tween 80 and 0.5% (v/v) methylcellulose in water. On the day of dosing, non-labeled DCDQ (205.7 mg) was dissolved in the vehicle to a final concentration of approximately 10 mg/mL.

Three male rats weighing from 413 to 474 grams and three female rats weighing from 272 to 290 grams at the time of dosing were purchased from Charles River Laboratories (Wilmington, Mass.). Non-fasted rats were given a single 50 mg/kg target dose of DCDQ at a volume of 5.0 mL/kg via intragastric gavage. Animals were provided standard rat chow and water ad libitum, and were kept in metabolism cages individually.

Urine was collected into containers on dry ice at 0-12 and 12-24 hour intervals, and stored at approximately −70° C. until fraction collection.

Analysis of Rat Urine by Liquid Chromatography/Mass Spectrometry

Rat urine samples were analyzed by LC/MS to characterize the DCDQ metabolites present in the rat urine samples used for metabolite isolation. The HPLC system used for LC/MS analysis was an Agilent Model 1100 HPLC system (Agilent Technologies, Palo Alto, Calif.) equipped with a binary pump, autosampler and diode array UV detector. The autosampler temperature was set to 10° C. The UV detector was set to monitor 190 to 400 nm. Separations were accomplished with a Supelco Discovery C18 column (250×2.1 mm×5 μm). The column temperature was 20° C. The mobile phase gradient program used was as described below.
Mobile phase A: 10 mM Ammonium acetate in water, pH 4.5

Mobile phase B: Methanol

TABLE 27
HPLC Gradient

| | Mobile Phase A | Mobile Phase B |
|---|---|---|
| Time (min) | (%) | (%) |
| 0 | 90 | 10 |
| 6 | 80 | 20 |
| 35 | 70 | 30 |
| 65 | 15 | 85 |
| 80 | 15 | 85 |
| 81 | 90 | 10 |
| 100 | 90 | 10 |

The mass spectrometer used for metabolite characterization was a Finnigan LCQ ion trap mass spectrometer (ThermoElectron Corp., San Jose, Calif.). It was equipped with an electrospray ionization (ESI) interface and operated in the positive ionization mode. Settings for the mass spectrometer are listed below.

TABLE 28
Finnigan LCQ Ion Trap Mass Spectrometer Settings

| Nebulizer gas | 90 arb. units |
|---|---|
| Auxiliary gas | 10 arb. units |
| Spray voltage | 3.5 KV |
| Heated capillary temp. | 200° C. |
| Full scan AGC setting | 4 × 10⁷ |
| Relative collision energy | 30% |

Metabolite Isolation by Liquid Chromatography

The HPLC system used for metabolite isolation consisted of a Waters Prep LC 4000 pump, a Waters 2767 Sample Manager for sample injection, Waters 996 diode array UV detector and a Gilson FC204 fraction collector (Gilson, Inc., Middleton, Wis.). The UV detector was set to monitor 210-450 nm. The fraction collector was set to collect fractions at 1 min intervals. The HPLC mobile phase gradient was as described above for LC/MS analysis except that the flow rate was 4.7 mL/min. Mobile phases were as described below for each HPLC Condition. No mass spectral analysis was conducted during fraction collection.

Two HPLC Conditions were used to isolate metabolites. HPLC Condition 1 was used to fractionate metabolites from rat urine. HPLC Condition 2 was used to further purify the DCDQ metabolite fractions collected using HPLC Condition 1. The columns and mobile phases used for HPLC Conditions 1 and 2 are listed below.

HPLC Condition 1

Column: Supelco Discovery C18 semi-preparative column (250×10 mm, 5 μm)
(Supelco, Bellefonte, Pa.).
Mobile phase A: 10 mM Ammonium acetate in water, pH 4.5
Mobile phase B: Methanol.
   HPLC Condition 2
Column: Zorbax SB-CN semi-preparative column (250×9.4 mm, 5 μm) (Agilent Technologies)
Mobile phase A: 0.02% Trifluoroacetic acid in water
Mobile phase B: 0.02% Trifluoroacetic acid in methanol

Fractions containing metabolites M7, M9 and M13 from HPLC Condition 2 were combined and evaporated to dryness under nitrogen using a Zymark TurboVap (Caliper Life Sciences, Hopkinton, Mass.). Dried metabolites were submitted for NMR spectroscopic analysis.

### Nuclear Magnetic Resonance (NMR) Spectroscopy Analysis of Isolated M7, M9 and M13

For NMR spectroscopy, samples of isolated DCDQ metabolites M7, M9 and M13 were each dissolved in 150 μL of 100% DMSO-d6 and transferred to individual 3 mm NMR tubes under a nitrogen gas atmosphere. One-dimensional (1 D) proton NMR and two-dimensional (2D) NMR (COSY, ROESY) data were collected on a Varian Inova 500 MHz NMR spectrometer (Palo Alto, Calif.) equipped with a Nalorac 5 mm z-gradient indirect detection probe (Varian).

### Data Analysis

ThermoFinnigan Xcalibur software (version 1.3) was used to control the LC/MS system and analyze LC/MS data. Micromass MassLynx software (version 4.0) was used for control of the HPLC equipment used for fraction collection. NMR spectroscopic data were collected, processed and displayed using the VNMR program (version 6.1C, Varian).

### Results

### DCDQ Metabolite Characterization by LC/MS and NMR Spectroscopy

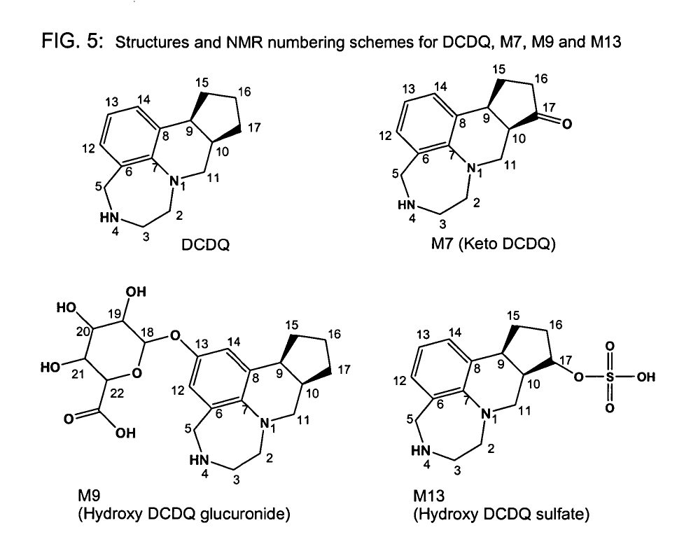

In this study, DCDQ metabolites M7, M9 and M13 were isolated in sufficient amounts to conduct NMR spectroscopic analysis for more detailed structural characterizations. The structural identifications for M7 (keto DCDQ), M9 (hydroxy DCDQ glucuronide) and M13 (hydroxy DCDQ sulfate) presented in this report shall replace those presented in previous reports. Structures of DCDQ, M7, M9 and M13, along with their NMR numbering schemes, are summarized in FIG. 5. Identification of M7, M9 and M13 by mass and NMR spectroscopy is discussed below.

### DCDQ

The mass spectral and NMR characteristics of DCDQ standard were examined for comparison with the metabolites. In the LC/MS spectrum of DCDQ, a protonated molecular ion, [M+H]⁺, was observed at m/z 229. The product ions of m/z 229 mass spectrum of DCDQ obtained from collision-induced dissociation (CID), and the proposed fragmentation scheme indicated a loss of methyleneamine and ethylideneamine from the molecular ion generated the product ions at m/z 200 and 186, respectively. Loss of propene from the molecular ion generated m/z 187 and further loss of ethylideneamine generated m/z 144. Loss of the cyclopentyl-methyleneamine group generated the m/z 132 product ion.

Table 29 summarizes the ¹HNMR chemical shift data for DCDQ. These data were used for comparison with the isolated metabolites.

TABLE 29
¹H Chemical Shifts for DCDQ in DMSO-d₆

| | δ¹H |
|---|---|
| Atom Number | (ppm)^{a,b} |
| 2 | 3.40, 3.10 |
| 3 | 3.19, 3.13 |
| 4 | 9.68, 8.77 (salt) |
| 5 | 4.20, 4.07 |
| 9 | 2.94 |
| 10 | 2.23 |
| 11 | 3.06, 2.66 |
| 12 | 7.18 |
| 13 | 6.91 |
| 14 | 7.24 |
| 15 | 2.19, 1.34 |
| 16 | 1.65, 1.55 |
| 17 | 2.00, 1.26 |

^{a}Chemical shifts are referenced to residual internal TMS (0.0 ppm) for ¹H.
^{b}Several proton assignments could not be made because of overlap in the 3.35 to 3.15 ppm region.

### Metabolite M7

The [M+H]⁺ for M7 was observed at m/z 243. The product ions of m/z 243 mass spectrum of M7 and the proposed fragmentation scheme indicated a loss of methyleneamine, ethylideneamine from the molecular ion generated the product ions at m/z 214 and 200, respectively, which were 14 Da larger than the corresponding ions at m/z 200 and 186, respectively, for DCDQ. This suggested the addition of one oxygen atom and loss of two hydrogen atoms from DCDQ. The product ions at m/z 132, 144 and 158 were the same as for DCDQ, which indicated the site of biotransformation as the cyclopentane ring.

Table 30 lists the chemical shifts and assignments for M7. The metabolite was assigned using information from the 1D NMR spectrum and the 2D COSY spectrum. The 1D ¹H NMRspectrum showed that the aromatic ring was intact with three aromatic resonances coupled in series. With the available data, it was not possible to distinguish H12 from H14. The protons in the dizaepine ring were assigned from the salt resonances (H4) at 9.10 and 8.62 ppm. The 2D COSY data showed that these protons were coupled to the protons at 3.20 ppm (H3) and 4.18 and 4.15 ppm (H5). The H3 protons were also coupled to protons at 3.34 and 3.06 (H2). These results confirmed that the dizaepine ring was intact.

TABLE 30
¹H Chemical Shifts for Metabolite M7 in DMSO-d₆

| | δ¹H |
|---|---|
| Atom Number | (ppm)^{a} |
| 2 | 3.34, 3.06 |
| 3 | 3.20 |
| 4 | 9.10, 8.62 |
| 5 | 4.18, 4.15 |
| 9 | 3.47 |
| 10 | 2.59 |
| 11 | 3.16, 3.01 |
| 12 | 7.23^{b} |
| 13 | 7.01 |
| 14 | 7.35^{b} |
| 15 | 2.53, 1.76 |
| 16 | 2.32, 2.14 |

^{a}Chemical shifts are referenced to residual internal DMSO-d₆ at 2.49 ppm for ¹H.
^{b}The assignments for H12 and H14 might be interchanged.

The 1D ¹H NMRspectrum also showed a change occurred in the cyclopentyl region versus DCDQ because there were three resonances upfield of 2.5 ppm while for DCDQ, there were seven resonances. The assignment of the remaining protons began with the H11 protons. The resonances at 3.16 ppm and 3.01 ppm were assigned to H11 based on comparison to the coupling constants observed for DCDQ. The resonance at 3.01 ppm was a triplet and the resonance at 3.16 ppm was a doublet of doublets. These were also observed for DCDQ. The H11 protons were both coupled to a resonance at 2.59 ppm (H10). The H10 resonance was coupled to one other resonance at 3.47 ppm (H9). The H9 resonance was coupled to a methylene pair at 2.53 ppm and 1.76 ppm (H15). The H15 resonances were coupled to another methylene pair at 2.32 ppm and 2.14 ppm (H16). There were no resonances assignable to H17, which indicated the site of metabolism as the C17 position. The downfield shift of the H16 protons would be consistent with a carbonyl oxygen at C17. Therefore, M7 was identified as 17-keto DCDQ.

### Metabolite M9

M9 generated a [M+H]⁺ at m/z 421. The product ions of m/z 421 mass spectrum of M9 and the proposed fragmentation scheme indicated a loss of 176 Da from the molecular ion generated m/z 245, 16 Da larger than the DCDQ molecular ion, which indicated glucuronidation of hydroxy DCDQ. Loss of ethylideneamine from [M+H]⁺ yielded m/z 378. This indicated an unchanged ethyleneamine moiety. Loss of glucuronic acid (176 Da) from m/z 378 yielded m/z 202, which was 16 Da higher than the corresponding ion at m/z 186 for DCDQ. This eliminated the three methylene groups of the cyclopropane ring as sites of metabolism. The product ion at m/z 203 was 16 Da larger than the corresponding ion at 187 for DCDQ. These data were consistent with either the benzyl or tetrahydropyridine group as the site of metabolism.

Table 31 lists the NMR chemical shifts and assignments for M9 using the numbering scheme in FIG. 5. Much of the metabolite could be assigned using information from the 1D NMR spectrum and results from 2D COSY analysis, showing the through-bond correlations in M9, and ROESY analysis, showing through-space NOE close contacts in M9, experiments. The resonances from the methylene protons on C2 were not assigned because of overlap. Their resonances were located between 3.35 ppm and 3.15 ppm. The upfield regions of the NMR spectra for M9 DCDQ were identical. Further analysis of this region using a COSY experiment confirmed that the pentyl ring system was intact. Also visible in the 1 D ¹H NMR spectrum for M9 were the resonances from the glucuronic acid. Several of these resonances could not be unambiguously assigned because of spectral overlap. There was an adequate amount of metabolite for the unassigned protons to resonate in the 3.35 to 3.15 ppm region.

TABLE 31
¹H Chemical Shifts for Metabolite M9 in DMSO-d₆

| | δ¹H |
|---|---|
| Atom Number | (ppm)^{a,b} |
| 3 | 2.19, 3.01 |
| 4 | 9.11, 8.55 |
| 5 | 4.18, 4.11 |
| 9 | 2.93 |
| 10 | 2.24 |
| 11 | 3.02, 2.62 |
| 12 | 6.94 |
| 14 | 6.91 |
| 15 | 2.19, 1.32 |
| 16 | 1.63, 1.52 |
| 17 | 2.00, 1.23 |
| 18 | 4.95 |
| 19 | 3.84 |
| 20 | 3.37 |

^{a}Chemical shifts are referenced to residual internal DMSO at 2.49 ppm for ¹H.
^{b}Several proton assignments could not be made because of overlap in the 3.35 to 3.15 ppm region.

Inspection of the 500 MHz 1 D ¹H NMR spectrum showed that the resonances for the aromatic region were changed from the NMR spectrum of DCDQ. Two of these aromatic resonances remained from the three for DCDQ. Coupling at 2.5 Hz between these aromatic resonances is characteristic of a meta orientation between the protons. This placed the glucuronic acid at the C13 position. The location of the glucuronic acid was further supported by the results of a ROESY experiment that showed H12 had NOEs to the H5 proton and H14 had an NOE to H9. These results positioned H12 and H14 at opposite ends of the aromatic ring. Both aromatic protons also had NOEs to the anomeric proton of the glucuronic acid ring. All these results were consistent with the glucuronic acid conjugation being at C13.

### Metabolite M13

The [M+H]⁺ for M13 was observed at m/z 325. The product ions of m/z 325 mass spectrum of M13 and the proposed fragmentation scheme indicated a loss of 80 Da from [M+H]⁺ yielded m/z 245, which was 16 Da larger than [M+H]⁺ for DCDQ. This indicated that M13 was a sulfate conjugate of hydroxy DCDQ. Loss of ethylideneamine from the molecular ion yielded m/z 282. The presence of product ions at m/z 144 and 132, also observed for DCDQ, indicated that one of the three methylene positions of the cyclopentane ring was the site of metabolism.

Table 32 lists the chemical shifts and assignments for M13. The metabolite was assigned using information from the 1 D ¹H NMR spectrum, 2D COSY spectrum and 2D ROESY spectrum. The 1D ¹H NMR spectrum for M13 showed that the aromatic ring was intact with three coupled protons at 7.15 ppm (Hi 2), 6.91 ppm (H13) and 7.23 ppm (H14). The assignments were confirmed by observing an ROE from H12 to the resonances at 4.17 ppm and 4.14 ppm which were identified as H5. The protons in the dizaepine ring were assigned from the salt resonances (H4) at 9.04 and 8.57 ppm. The 2D COSY data showed that these protons were coupled to the protons at 3.22 ppm and 3.20 ppm (H3) and 4.17 and 4.14 ppm (H5). The H3 protons were also coupled to protons at 3.34 and 3.14 (H2). These results confirmed that the dizaepine ring was intact.

TABLE 32
¹H Chemical Shifts for Metabolite M13 in DMSO-d₆

| | δ¹H |
|---|---|
| Atom Number | (ppm)^{a} |
| 2 | 3.34, 3.14 |
| 3 | 3.22, 3.20 |
| 4 | 9.10, 8.62 |
| 5 | 4.17, 4.14 |
| 9 | 3.15 |
| 10 | 2.40 |
| 11 | 3.23, 2.68 |
| 12 | 7.15 |
| 13 | 6.91 |
| 14 | 7.23 |
| 15 | 2.26, 1.33 |
| 16 | 1.97, 1.67 |
| 17 | 4.29 |

^{a}Chemical shifts are referenced to residual internal DMSO at 2.49 ppm for ¹H.

The 1 D ¹H NMR spectral data (Table 32) showed that a change occurred in the cyclopentyl region because there were five resonances upfield of 2.5 ppm for M13 while in the DCDQ NMR spectrum, there were seven resonances. The protons at position 11 were assigned based on their similarity to those for DCDQ. The triplet resonance at 2.68 ppm was unique to DCDQ. This resonance was coupled to a resonance at 3.23 ppm (H11) and another at 2.40 ppm (H10). H10 was coupled to a resonance at 3.15 ppm (H9) and weakly coupled to 4.29 ppm (H17). H9 was coupled to a methylene pair at 2.26 ppm and 1.33 ppm (both H15). This methylene pair was coupled to a second methylene pair at 1.97 ppm and 1.67 ppm (both H16). The H16 methylene was coupled to H17. One proton was missing from the cyclopentane ring and the large downfield shift of the remaining proton was indicative of a nearby heteroatom. All these data were consistent with the sulfate group present at the C17 position. Therefore, M13 was identified as 17-hydroxy DCDQ sulfate.

Discussion

The present study was designed to obtain rat urine for metabolite isolation and to obtain more specific structural identification for selected metabolites of DCDQ. Three male and three female rats were given a single 50 mg/kg dose of DCDQ. Urine was collected at 0-12 and 12-24 hour intervals. DCDQ metabolites M7 (keto DCDQ), M9 (hydroxyl DCDQ glucuronide) and M13 (hydroxyl DCDQ sulfate) were isolated from the urine by a two stage semi-preparative HPLC method in low microgram quantities sufficient for NMR spectroscopic analysis. Based upon MS and NMR spectroscopic analysis the site of metabolism for M7 and M13 was at 17 position 17. The site of metabolism for M9 was at position 13. The structural identifications for M7, M9 and M13 identified through this study further refine those of the in vivo rat study discussed above.

### In Vivo Metabolism of [¹⁴C] DCDQ in Healthy Human Subjects Following Oral Administration

The metabolite profiles of DCDQ in plasma and urine of healthy human subjects receiving a single or multiple oral doses of DCDQ at various dosages were determined. In addition, relative concentrations of the major DCDQ metabolite (M6, carbarmoyl glucuronide) were determined in selected samples.

DCDQ and several DCDQ metabolites were identified in plasma and urine. DCDQ carbamoyl glucuronide (M6) was the predominant drug-related component in both plasma and urine. DCDQ imine N-oxide (M5), unchanged DCDQ, DCDQ imine (P3) and other relatively minor drug-related components were also observed in plasma. Unchanged DCDQ, DCDQ N-oxide glucuronide (M40), hydroxyl DCDQ glucuronide (M38), hydroxyl DCDQ carbamoyl glucuronide (M37) and a number of other relatively minor drug-related components were excreted in urine.

The concentrations of M6 in plasma increased with increased dosage, and large individual variations were observed. Plasma M6 concentrations decreased over time from 6 to 24 hour post-dose. The ratios of M6-to-DCDQ plasma concentrations were higher at 6 hour than at 12 and 24 hour post-dose. At 6 hour post-dose, the average ratios ranged from 35.4 to 76.6. There were no statistically significant differences in M6 concentrations and the M6-to-DCDQ ratios between fasted and fed subjects receiving 300 mg of DCDQ. The average M6-to-DCDQ ratios ranged from 84 to 1018 in urine.

The results show that DCDQ underwent phase I and phase II metabolism in healthy human subjects receiving DCDQ orally, and carbamoyl glucuronidation was the major metabolic pathway. In contrast to animal studies, formation of the carbamoyl glucuronide (M6) was the major metabolic pathway in humans, and M6 was the predominant drug-related metabolite in human plasma and urine.

### Materials and Methods

### Methods

DCDQ hydrochloride with a chemical purity of 98.6% was synthesized by Wyeth Research (Pearl River, N.Y.). DCDQ carbamoyl glucuronide was synthesized by Chemical Development at Wyeth Research (Montreal, Canada), and had a purity of 95.5%. The internal standard (d₈-DCDQ, lot L27347-140-A) was synthesized by the Radiosynthesis group at Wyeth Research (Pearl River, N.Y.). The reported deuterium distribution was d₀-d₅ 0%, d₆ 0.1%, d₇ 2.7%, and d₈ 97.1%. Solvents used for extraction and for chromatographic analysis were HPLC or ACS reagent grade from EMD Chemicals (Gibbstown, N.J.).

### Methods

### Drug Administration and Specimen Collection

Drug administration and specimen collection were performed in a randomized, double-blinded, placebo-controlled, ascending single dose study of the safety, tolerability, pharmacokinetics, and pharmacodynamics of DCDQ administered orally to healthy subjects and subjects with schizophrenia and schizoaffective disorder. The specimens were stored at approximately −70° C. until analysis for metabolite profiles and for ratios of carbamoyl glucuronide (M6) to DCDQ.

### Sample Preparation

Two subjects with medium and high exposure to DCDQ in the 25 mg multiple ascending dose study were also analyzed by LC/MS for metabolite profiles. The 8 hr plasma samples collected on day 1 and day 14 from subjects 9 and 41 were processed and analyzed as described below. No internal standard was added to the samples analyzed for metabolite profiles.

Plasma samples from fasted subjects 25, 28 and 30 in the 50 mg single dose group, fasted subjects 50, 51, 54 in the 200 mg single dose group, fasted subjects 74, 76, 79 in the 300 mg single dose group, fed subjects 83, 84, 86 in the 300 mg single dose group and fasted subjects 92, 94, 96 in the 500 mg single dose group were analyzed for DCDQ carbamoyl glucuronide (M6) concentrations. The internal standard d8-DCDQ (25 μL of 200 ng/mL methanol solution) was added to 100 μL of the plasma samples, followed by the addition of 300 μL of acetonitrile. The samples were mixed and centrifuged at 14000 rpm in an Eppendorf 5415C centrifuge (Brinkman Instruments Inc., Westbury, N.Y.) for 10 minutes. The supernatant of each sample was transferred to a clean tube and evaporated to dryness under a stream of nitrogen in a TurboVap LV evaporator (Caliper Life Sciences, Hopkinton, Mass.). The residue was reconstituted with 50 μL of methanol followed by the addition of 150 μL of water. The sample was mixed and centrifuged as described above. The supernatant was analyzed by LC/MS/MS analysis. Samples for standard curves were prepared with control plasma spiked with synthetic M6. The concentrations of M6 used for the standard curve ranged from 0 to 2500 ng/mL plasma.

The 0-4, 4-12 and 12-24 hr urine samples from the same subjects in the single dose groups were analyzed for ratios of M6 to DCDQ. The internal standard was not used in the analysis of urine samples. The samples were diluted for 20-fold with a control urine sample and directly analyzed by LC/MS. To estimate M6-to-DCDQ ratios in human urine, control urine samples were spiked with 200 ng/mL of DCDQ and 1000, 5000, or 10000 ng/mL of DCDQ carbamoyl glucuronide, and were analyzed by LC/MS.

### Liquid Chromatography/Mass Spectrometry

Three LC/MS systems were used in this work. LC/MS System 1 was used for analysis of plasma and urine samples for metabolite characterization. LC/MS System 2 was used to provide additional MS/MS data for characterization of DCDQ metabolites in urine. LC/MS System 3 was used for semi-quantitative analysis of metabolite M6 (DCDQ carbamoyl glucuronide) in plasma and urine samples.

### LC/MS System 1

LC/MS System 1 was used for analysis of plasma and urine samples for metabolite characterization. The HPLC equipment used with this LC/MS System consisted of an Agilent Model 1100 HPLC system (Agilent Technologies, Palo Alto, Calif.) including an autosampler, binary pump and diode array UV detector. The UV detector was set to monitor 210 to 350 nm. The HPLC mobile phase consisted of 10 mM ammonium acetate, pH 4.5 (A) and methanol (B), and was delivered at 0.2 mL/min. The linear mobile phase gradient (HPLC Gradient 1) is shown below. During LC/MS sample analysis, up to 6 min of the initial flow was diverted away from the mass spectrometer prior to evaluation of metabolites.

HPLC Gradient 1

| Time (min) | A (%) | B (%) |
|---|---|---|
| 0 | 95 | 5 |
| 3 | 95 | 5 |
| 3.1 | 90 | 10 |
| 13 | 90 | 10 |
| 25 | 80 | 20 |
| 50 | 70 | 30 |
| 70 | 5 | 95 |
| 80 | 5 | 95 |

The mass spectrometer used for metabolite characterization with LC/MS System 1 was a Finnigan LCQ-Deca ion trap mass spectrometer (Thermo Electron, San Jose, Calif.). This mass spectrometer was equipped with an electrospray ionization (ESI) interface and operated in the positive ionization mode. Settings for the LCQ mass spectrometer are listed below.

Finnigan LCQ Mass Spectrometer Settings

| Spray voltage | 4.5 kV |
|---|---|
| Heated capillary temp. | 200° C. |
| Nebulizer gas pressure | 50 psi |
| Auxiliary gas setting | 60 |
| Full scan AGC setting | 5 × 10⁷ |
| Relative collision energy | 30% |

### LC/MS System 2

LC/MS System 2 was used to provide additional MS/MS data for characterization of DCDQ metabolites in urine. The HPLC equipment used with LC/MS System 2 consisted of a Waters model 2695 HPLC system (Waters Corp., Milford, Mass.). It was equipped with a built-in autosampler and a model 996 diode array UV detector. The UV detector was set to monitor 210-400 nm. The HPLC column, mobile phases, flow rate, diversion of flow away from the mass spectrometer and gradient were as described above for LC/MS System 1. The column temperature was 25° C.

The mass spectrometer used for metabolite characterization with LC/MS System 2 was a Micromass Quattro Micro triple quadrupole mass spectrometer (Waters Corp.). This mass spectrometer was equipped with an electrospray interface and operated in the positive ionization mode. Settings for this mass spectrometer are listed below.

Micromass Mass Spectrometer Settings

| | |
|---|---|
| ESI spray | 2.5 kV |
| Cone | 45 V |
| Mass resolution (MS1 and MS2) | 0.7 Da ± 0.2 Da width at half height |
| Desolvation gas flow | 900-1100 L/hr |
| Cone gas flow | 50-70 L/hr |
| Source block temp. | 80° C. |
| Desolvation gas temp. | 200° C. |
| Collision gas pressure | 1.0-1.2 × 10⁻³ mbar |
| Collision offset | 22 eV |

### LC/MS System 3

LC/MS System 3 was used for semi-quantitative analysis of metabolite M6 (DCDQ carbamoyl glucuronide) in plasma and urine samples. The HPLC equipment for this LC/MS System consisted of a Thermo Surveyor HPLC (Thermo Electron Corp., San Jose, Calif.), including a Surveyor MS pump and autosampler. Separations were accomplished on a 5 micron Phenomenex Luna C18(2) column, 150×2 mm (Phenomenex, Torrance, Calif.). The autosampler and column temperatures were set at 5° C. and 40° C., respectively. The HPLC mobile phase consisted of 10 mM ammonium acetate (A) and methanol (B), and was delivered at 0.2 mL/min. The linear mobile phase gradient (HPLC Gradient 2) is shown below. During LC/MS sample analysis, up to 3 min of the initial flow was diverted away from the mass spectrometer prior to evaluation of metabolites.

HPLC Gradient 2

| Time (min) | A (%) | B (%) |
|---|---|---|
| 0 | 90 | 10 |
| 3 | 90 | 10 |
| 11 | 10 | 90 |
| 18 | 10 | 90 |
| 20 | 90 | 10 |
| 24 | 90 | 10 |

The mass spectrometer used for semi-quantitative anslysis with LC/MS System 3 was a Finnigan TSQ Quantum triple quadrupole mass spectrometer (Thermo Electron Corp.). The mass spectrometer was equipped with an electrospray interface and operated in the positive ionization mode. Settings for this mass spectrometer are listed below.

Finnigan TSQ Quantum Mass Spectrometer Settings

| | |
|---|---|
| Spray voltage | 4.5 kV |
| Capillary temperature | 250° C. |
| Q1 mass resolution setting | 0.8 Da width at half height |
| Q3 mass resolution setting | 0.6 Da width at half height |
| Nebulizer gas pressure | 30 arb. units |
| Auxiliary gas setting | 50 arb. units |
| Collision gas pressure | 1.5 mtorr |

LC/MS/MS analysis in the selected reaction monitoring (SRM) mode (LC/SRM) was conducted for DCDQ and M6 using the following settings.

LC/SRM Analysis Settings

| | Nominal Mass | | Collision offset | Dwell time |
|---|---|---|---|---|
| Compound | Q1 | Q3 | (eV) | (ms) |
| DCDQ | 229 | 186 | 22 | 300 |
| d₈-DCDQ | 237 | 194 | 22 | 300 |
| (plasma only) | | | | |
| DCDQ carbamoyl | 449 | 273 | 25 | 300 |
| glucuronide | | | | |
| (M6) | | | | |

### Data Analysis and Statistical Evaluation

The computer program Microsoft Excel® 97 was used to calculate means and standard deviations and to perform the student t-test. Xcalibur (version 1.3) and MassLynx software (version 4.0) were used for collection and analysis of LC/MS data. Peak area ratios of M6 to the internal standard were used for quantitation of M6 in plasma samples.

### Results

### Metabolite Profiles and M6 Concentrations in Plasma

DCDQ and eight DCDQ metabolites were identified in human plasma (Table 33). DCDQ carbamoyl glucuronide (M6) was the predominant drug-related component in plasma in all dose groups in both single and multiple dose studies. DCDQ imine N-oxide (M5), unchanged DCDQ, DCDQ imine (P3) and trace amounts of hydroxyl DCDQ, hydroxyl DCDQ imine, hydroxyl DCDQ glucuronide (M9) and keto DCDQ glucuronide (M22) were also observed in plasma. Metabolite profiles were qualitatively similar in all samples analyzed.

Plasma M6 concentrations increased with increased dosage, and large individual variations were observed (Table 34). Of the three time points analyzed, M6 concentrations were the highest at 6 hour post-dose and decreased over time at 12 and 24 hour post-dose. The ratios of M6 to DCDQ plasma concentrations were also the highest at 6 hour post-dose, and in general decreased over time. At 6 hour post-dose, the average ratios ranged from 35.4 to 76.6. There were no statistically significant differences in M6 concentrations and the M6 to DCDQ ratios between fasted and fed subjects receiving 300 mg of DCDQ.

### Metabolite Profiles and M6 to DCDQ Ratios in Urine

DCDQ and several DCDQ metabolites were identified in urine. DCDQ carbamoyl glucuronide (M6) was the predominant drug-related component in urine, as in plasma. Unchanged DCDQ, DCDQ N-oxide glucuronide (M40), hydroxyl DCDQ glucuronide (M38), hydroxyl DCDQ carbamoyl glucuronide (M37) and trace amounts of DCDQ imine (P3), hydroxyl DCDQ (M1 and M32), hydroxyl DCDQ imine (M29), keto DCDQ glucuronide (M22), hydroxyl DCDQ glucuronide (M9), hydroxyl DCDQ carbamoyl glucuronides (M33, M36 and M39), DCDQ imine glucuronide (M34) and dihydroxyl DCDQ imine glucuronide (M35) were also observed in urine. The carbamoyl glucuronide (M6) was present in urine at much higher concentrations than the parent drug (Table 35). The average M6 to DCDQ ratios ranged from 84 to 1018; large variations were observed. The ratios appeared to be lower in the 500 mg dosage group than in the other dose groups.

### Metabolite Characterization by Liquid Chromatography/Mass Spectrometry

Mass spectra were obtained by LC/MS and LC/MS/MS analysis for DCDQ and its metabolites in human plasma and urine. Table 33 summarizes the DCDQ metabolites characterized in this study. As M6 (DCDQ carbamoyl glucuronide) was the predominant DCDQ related component in both plasma and urine, and the relative concentration of unchanged DCDQ was relatively minor. Therefore, mass spectral characterization only of DCDQ related components present in approximately equal or greater concentrations than unchanged DCDQ in plasma or urine are discussed in more detail below.

### DCDQ

The mass spectral characteristics of DCDQ authentic standard were examined for comparison with metabolites. In the LC/MS spectrum of DCDQ, a protonated molecular ion, [M+H]⁺, was observed at m/z 229. Loss of NH₃ from [M+H]⁺ yielded m/z 212. Loss of methyleneamine, ethylideneamine and propylideneamine from the molecular ion generated the product ions at m/z 200, 186 and 171, respectively. Loss of the propene group from the molecular ion generated m/z 187 and further loss of methyleneamine and ethylideneamine yielded m/z 158 and 144. Loss of cyclopentene from [M+H]⁺ yielded m/z 161. Loss of the cyclopentyl-methyleneamine group generated the product ion at m/z 132.

### Metabolite M5

Metabolite M5 produced a [M+H]⁺ at m/z 243, which was 14 Da larger than DCDQ and 16 Da larger than P3. These data suggested that M5 was a keto DCDQ metabolite, hydroxyl DCDQ imine or DCDQ imine N-oxide. Loss of NH₃ and H₂O from [M+H]⁺ yielded m/z 226 and 225, respectively, which was consistent with addition of an oxygen atom. Loss of methyleneamine, from the molecular ion was proposed to generate m/z 213, consistent with addition of oxygen and the presence of a double bond from loss of two hydrogens. The product ion at m/z 130 was also observed for P3 and was 2 Da less than the corresponding ion at m/z 132 for DCDQ. These data indicated that M5 also contained an imine group, which eliminated keto DCDQ from consideration. The HPLC retention time of M5 was longer than both DCDQ and P3 (DCDQ imine), which was also observed in the in vitro metabolism study¹ and consistent with N-oxidation. Therefore, M5 was identified as DCDQ imine N-oxide.

### Metabolite M6

The [M+H]⁺ for M6 was observed at m/z 449, which was 220 Da larger than DCDQ. Loss of 176 Da from the molecular ion generated m/z 273, indicating that M6 was a glucuronide. Further loss of 44 Da from m/z 273 yielded m/z 229, which was also the molecular ion for DCDQ. Product ions at m/z 212 and 186 were also observed for DCDQ, consistent with M6 being a conjugate of DCDQ. Therefore, M6 was identified as the carbamoyl glucuronide of DCDQ.

### Metabolite M38

The [M+H]⁺ for M38 was observed at m/z 421, which was 192 Da larger than DCDQ. Loss of NH₃ from [M+H]⁺ yielded m/z 404, which suggested an unchanged amino group on the diazepane ring. Loss of 176 Da from the molecular ion generated m/z 245, which was also the molecular ion for hydroxyl DCDQ metabolites. Losses of NH₃ and H₂O from m/z 245 generated m/z 228 and 227, respectively. These data indicated that M38 was a glucuronide of a hydroxyl DCDQ. The product ion at m/z 362 was 176 Da larger than the corresponding ion at m/z 186 for DCDQ, which indicated glucuronidation of the quinoline-cyclopentane moiety. Product ions at m/z 362 and 269 were proposed to include the glucuronic acid moiety and have been the result of fragmentation of diazepine, quinoline and cyclopentane rings as indicated in the fragmentation scheme. These data were consistent with glucuronidation of the quinoline nitrogen and hydroxylation of the diazepine ring. Therefore M38 was identified as a hydroxyl DCDQ glucuronide.

### Metabolite M40

The [M+H]⁺ for M40 was observed at m/z 421, which was 192 Da larger than DCDQ. Loss of H₂O from [M+H]⁺ yielded m/z 403. No apparent loss of NH₃ was observed from [M+H]⁺, which suggested a modified amino group on the diazepane ring. Loss of 176 Da from the molecular ion generated m/z 245, which was also the molecular ion for hydroxyl DCDQ metabolites. However, the relative intensity m/z 245 for M40 was weaker than was observed for metabolite M38. Loss of an oxygen atom from m/z 245 yielded m/z 229, also the molecular ion for DCDQ. These data in combination with the presence of m/z 229 as the base peak in the ion trap mass spectrum, rather than m/z 245 as was observed for metabolite M38, indicated the presence of an N-oxide. Product ions at m/z 228, 227, 212 and 210 respectively were generated by losses of H₂O and NH₃ from m/z 245 and 229 as indicated in the fragmentation scheme. The HPLC retention time of M40 was longer than for M38, which was also consistent with M40 being an N-oxide. Product ions at m/z 200 and 186 were also observed for DCDQ and were proposed to be the result of loss of an oxygen atom from the corresponding N-oxide product ions for M40. Product ions at m/z 360 and 271 were proposed to include the glucuronic acid moiety and have been the result of fragmentation of diazepine, quinoline and cyclopentane rings as indicated in the fragmentation scheme. These data were consistent with glucuronidation of the amino group of the diazepine ring and N-oxidation of the quinoline nitrogen. Therefore, M40 was proposed to be a DCDQ N-oxide glucuronide.

### Discussion

DCDQ underwent metabolism in humans. DCDQ carbamoyl glucuronide (M6) was the predominant drug-related component in both plasma and urine. DCDQ imine N-oxide (M5), unchanged DCDQ, DCDQ imine (P3) were the other major drug-related components observed in plasma. Unchanged DCDQ, DCDQ N-oxide glucuronide (M40), hydroxyl DCDQ glucuronide (M38), hydroxyl DCDQ carbamoyl glucuronide (M37) were excreted in urine.

Plasma M6 concentrations increased with increased dosage, and large individual variations were observed. M6 concentrations decreased over time from 6 to 24 hour post-dose. The ratios of M6 to DCDQ plasma concentrations were higher at 6 hour than at 12 and 24 hour post-dose. At 6 hour post-dose, the average ratios ranged from 35.4 to 76.6. In contrast, much lower amounts of M6 were detected in the previous in vitro and in vivo studies. There were no statistically significant differences in M6 concentrations and the M6 to DCDQ ratios between fasted and fed subjects receiving 300 mg of DCDQ. The average M6-to-DCDQ ratios ranged from 84 to 1018 in urine. In summary, DCDQ underwent both phase I and phase 11 metabolism in healthy human subjects and carbamoyl glucuronidation was the major metabolic pathway. In contrast to animal studies, formation of the carbamoyl glucuronide (M6) was the major metabolic pathway in humans, and M6 was the predominant drug-related metabolite in human plasma and urine.

TABLE 33
DCDQ metabolites characterized by LC/MS in human plasma and urine

| | Metabolite | | | | |
|---|---|---|---|---|---|
| t_{R}^{a} | peak | [M + H]⁺ | Site of metabolism | Name | Matrix^{b} |
| 31.7 | M32 | 245 | Cyclopropane or | Hydroxyl DCDQ | P, U |
| | | | Piperidine ring | | |
| 32.2 | M29 | 243 | Cyclopentane and | Hydroxyl DCDQ imine | P, U |
| | | | diazepane rings | | |
| 36.3 | M1 | 245 | Piperidine ring | Hydroxyl DCDQ | P, U |
| 38.3 | M22 | 419 | Diazapine and | Keto DCDQ glucuronide | P, U |
| | | | Cyclopentane rings | | |
| 49.6 | M9 | 421 | Benzene ring | Hydroxyl DCDQ | P, U |
| | | | | Glucuronide | |
| 58.0 | M33 | 465 | Diazepine ring | Hydroxyl DCDQ | U |
| | | | | carbamoyl glucuronide | |
| 63.3 | M34 | 403 | Diazepine ring | DCDQ imine glucuronide | U |
| 63.6 | M35 | 435 | Diazepine and | Dihydroxyl DCDQ imine | U |
| | | | cyclopentane rings | glucuronide | |
| 64.2 | M36 | 465 | Diazepine ring and | Hydroxyl DCDQ | U |
| | | | quinoline or cyclopenatne | carbamoyl glucuronide | |
| | | | ring | | |
| 65.9 | P3 | 227 | Diazepine ring | DCDQ imine | P, U |
| 67.1 | M37 | 465 | Diazepine ring and | Hydroxyl DCDQ | U |
| | | | quinoline or cyclopenatne | carbamoyl glucuronide | |
| | | | ring | | |
| 68.6 | DCDQ | 229 | None | DCDQ | P, U |
| 70.9 | M38 | 421 | Quinoline nitrogen and | Hydroxyl DCDQ | U |
| | | | diazepine ring | glucuronide | |
| 71.4 | M5 | 243 | Diazepine ring | DCDQ imine N-oxide | P |
| 71.4 | M39 | 465 | Diazepine ring and | Hydroxyl DCDQ | U |
| | | | quinoline or cyclopenatne | carbamoyl glucuronide | |
| | | | ring | | |
| 73.6 | M40 | 421 | Diazepine nitrogens | DCDQ N-oxide | U |
| | | | | glucuronide | |
| 75.2 | M6 | 449 | Diazepine secondary | Carbamoyl Glucuronide | P, U |
| | | | amine group | of DCDQ | |

^{a}Retention times taken from LC/MS data
^{b}P = plasma, U = urine

TABLE 34
Concentrations (ng/mL) of DCDQ and DCDQ Carbamoyl
Glucuronide (M6) in Plasma from Healthy Subjects
Receiving DCDQ

| | | | | | M6 to |
|---|---|---|---|---|---|
| Dose | Time | | | | DCDQ |
| (mg) | (hr) | Subject | DCDQ^{a} | M6 | Ratios |
| 50 | 6 | 25 | 1.5 | 83.1 | 55.4 |
| (fasted) | | 28 | 0.9 | 127 | 141 |
| | | 30 | 5.0 | 132 | 26.4 |
| | | Mean ± S.D. | 2.47 ± 2.21 | 114 ± 26.9 | 74.3 ± 59.6 |
| | 12 | 25 | 0.7 | 15.1 | 21.6 |
| | | 28 | 0.4 | 6.60 | 16.5 |
| | | 30 | 2.5 | 15.4 | 6.16 |
| | | Mean ± S.D. | 1.20 ± 1.14 | 12.4 ± 5.00 | 14.8 ± 7.87 |
| | 24^{b} | 25 | 0.2 | 3.1 | 15.5 |
| | | 28 | NA^{c} | NA | NA |
| | | 30 | 0.6 | 3.6 | 6.00 |
| | | Mean | 0.40 | 3.35 | 10.8 |
| 200 | 6 | 50 | 2.7 | 225 | 83.3 |
| (fasted) | | 51 | 18.0 | 615 | 34.2 |
| | | 54 | 12.0 | 216 | 18.0 |
| | | Mean ± S.D. | 10.9 ± 7.71 | 352 ± 228 | 45.2 ± 34.0 |
| | 12 | 50 | 1.2 | 37.5 | 31.3 |
| | | 51 | 9.2 | 62.9 | 6.84 |
| | | 54 | 6.4 | 30.3 | 4.73 |
| | | Mean ± S.D. | 5.60 ± 4.06 | 43.6 ± 17.1 | 14.3 ± 14.8 |
| | 24 | 50 | 0.3 | 4.8 | 16.0 |
| | | 51 | 2.6 | 8.8 | 3.38 |
| | | 54 | 1.6 | 9.4 | 5.88 |
| | | Mean ± S.D. | 1.50 ± 1.15 | 7.67 ± 2.50 | 8.42 ± 6.68 |
| 300 | 6 | 74 | 2.09 | 123 | 58.9 |
| (fasted) | | 76 | 4.27 | 249 | 58.3 |
| | | 79 | 16.0 | 339 | 21.2 |
| | | Mean ± S.D. | 7.45 ± 7.48 | 237 ± 108 | 46.1 ± 21.6 |
| | 12 | 74 | 1.50 | 43.0 | 28.7 |
| | | 76 | 3.34 | 78.0 | 23.4 |
| | | 79 | 7.84 | 145 | 18.5 |
| | | Mean ± S.D. | 4.23 ± 3.26 | 88.7 ± 51.8 | 23.5 ± 5.10 |
| | 24 | 74 | 0.972 | 48.5 | 49.9 |
| | | 76 | 1.72 | 12.0 | 6.98 |
| | | 79 | 1.70 | 26.3 | 15.5 |
| | | Mean ± S.D. | 1.46 ± 0.43 | 28.9 ± 18.4 | 24.1 ± 22.7 |
| 300 | 6 | 83 | 19.0 | 860 | 45.3 |
| (fed) | | 84 | 15.6 | 696 | 44.6 |
| | | 86 | 1.30 | 182 | 140 |
| | | Mean ± S.D. | 12.0 ± 9.39 | 579 ± 354 | 76.6 ± 54.9 |
| | 12 | 83 | 10.9 | 279 | 25.6 |
| | | 84 | 8.06 | 38.1 | 4.73 |
| | | 86 | 5.97 | 540 | 90.5 |
| | | Mean ± S.D. | 8.31 ± 2.47 | 286 ± 251 | 40.3 ± 44.7 |
| | 24 | 83 | 2.60 | 24.6 | 9.46 |
| | | 84 | 1.15 | 6.40 | 5.57 |
| | | 86 | 2.69 | 146 | 54.3 |
| | | Mean ± S.D. | 2.15 ± 0.86 | 59.0 ± 75.9 | 23.1 ± 27.1 |
| 500 | 6 | 92 | 44.5 | 1324 | 29.8 |
| (fasted) | | 94 | 31.7 | 508 | 16.0 |
| | | 96 | 20.2 | 1221 | 60.4 |
| | | Mean ± S.D. | 32.1 ± 12.2 | 1018 ± 444 | 35.4 ± 22.7 |
| | 12 | 92 | 24.5 | 146 | 5.96 |
| | | 94 | 21.7 | 181 | 8.34 |
| | | 96 | 12.0 | 206 | 17.2 |
| | | Mean ± S.D. | 19.4 ± 6.56 | 178 ± 30.1 | 10.5 ± 5.92 |
| | 24 | 92 | 5.53 | 58.3 | 10.5 |
| | | 94 | 6.88 | 13.5 | 1.96 |
| | | 96 | 3.95 | 77.0 | 19.5 |
| | | Mean ± S.D. | 5.45 ± 1.47 | 49.6 ± 32.6 | 10.7 ± 8.77 |

^{a}DCDQ concentrations were determined by Bioanalytical with a validated assay for human plasma.⁴ Concentrations of M6 were quantified by anon-validated LC/MS method in Biotransformation using a standard curve generated with synthesized M6.
^{b}Standard deviation was not calculated;
^{c}NA, not analyzed because the plasma DCDQ level was below the level of quantitation.

TABLE 35
Concentrations (ng/mL) of DCDQ and DCDQ
Carbamoyl Glucuronide (M6) to DCDQ Ratios in Urine from
Healthy Subjects Receiving DCDQ

| Dose | Time | | | M6 to DCDQ |
|---|---|---|---|---|
| (mg) | (hr) | Subject | DCDQ^{a} | Ratios |
| 50 | 0-4 | 25 | 13.7 | 571 |
| (fasted) | | 28 | 13.7 | 1739 |
| | | 30 | 115 | 214 |
| | | Mean ± S.D. | 47.5 ± 58.5 | 841 ± 798 |
| | 4-12 | 25 | 31 | 389 |
| | | 28 | 18.2 | 606 |
| | | 30 | 126 | 261 |
| | | Mean ± S.D. | 58.4 ± 58.9 | 419 ± 174 |
| | 12-24 | 25 | 17.7 | 143 |
| | | 28 | 5.21 | 259 |
| | | 30 | 75.3 | 63.9 |
| | | Mean | 32.7 ± 37.4 | 155 ± 98.1 |
| 200 | 0-4 | 50 | 251 | 169 |
| (fasted) | | 51 | 288 | 171 |
| | | 54 | 57 | 113 |
| | | Mean ± S.D. | 199 ± 124 | 151 ± 32.9 |
| | 4-12 | 50 | 79.5 | 397 |
| | | 51 | 177 | 240 |
| | | 54 | 97.3 | 215 |
| | | Mean ± S.D. | 118 ± 51.9 | 284 ± 98.7 |
| | 12-24 | 50 | 23.1 | 216 |
| | | 51 | 181 | 85.9 |
| | | 54 | 137 | 42.3 |
| | | Mean ± S.D. | 114 ± 81.5 | 115 ± 90.4 |
| 300 | 0-4 | 74 | 9.43 | 1167 |
| (fasted) | | 76 | 62.8 | 852 |
| | | 79 | 172 | 440 |
| | | Mean ± S.D. | 81.4 ± 82.9 | 820 ± 365 |
| | 4-12 | 74 | 21.7 | 329 |
| | | 76 | 93.4 | 1543 |
| | | 79 | 72.3 | 324 |
| | | Mean ± S.D. | 62.5 ± 36.8 | 732 ± 702 |
| | 12-24 | 74 | 22.9 | 485 |
| | | 76 | 298 | 127 |
| | | 79 | 64.1 | 110 |
| | | Mean ± S.D. | 128 ± 148 | 241 ± 212 |
| 300 | 0-4 | 83 | 55.9 | 1162 |
| (fed) | | 84 | 1.56 | 441 |
| | | 86 | 1.82 | 1451 |
| | | Mean ± S.D. | 19.8 ± 31.3 | 1018 ± 520 |
| | 4-12 | 83 | 259 | 375 |
| | | 84 | 174 | 571 |
| | | 86 | 30.6 | 1337 |
| | | Mean ± S.D. | 155 ± 115 | 761 ± 508 |
| | 12-24 | 83 | 351 | 148 |
| | | 84 | 116 | 92.6 |
| | | 86 | 308 | 184 |
| | | Mean ± S.D. | 258 ± 125 | 142 ± 46.0 |
| 500 | 0-4 | 92 | 1569 | 71.5 |
| (fasted) | | 94 | 835 | 60.2 |
| | | 96 | 339 | 161 |
| | | Mean ± S.D. | 914 ± 619 | 97.6 ± 55.2 |
| | 4-12 | 92 | 1683 | 149 |
| | | 94 | 612 | 34.6 |
| | | 96 | 1811 | 69.4 |
| | | Mean ± S.D. | 1369 ± 658 | 84.3 ± 58.6 |
| | 12-24 | 92 | 160 | 288 |
| | | 94 | 405 | 36.0 |
| | | 96 | 273 | 203 |
| | | Mean ± S.D. | 279 ± 123 | 176 ± 128 |

^{a}DCDQ concentrations were determined by Bioanalytical with a validated assay for human urine. ⁵ Concentrations of M6 were quantified by an non-validated LC/MS method in Biotransformation using a standard curve generated with synthesized M6.

All references, including but not limited to articles, texts, patents, patent applications, publications, and books, cited herein are hereby incorporated by reference in their entirety. This application claims priority benefit of U.S. Provisional Application Ser. No. 60/625,335 filed Nov. 5, 2004, the entire content of which is incorporated by reference herein in its entirety.

What is claimed is:

1. A compound of formula I 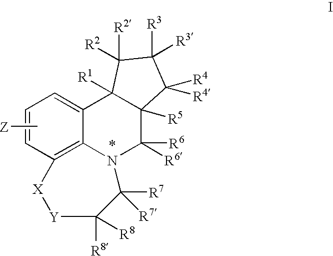
wherein:
for each Rⁿ and R^{n′}, where n is 1 through 8:
each Rⁿ and R^{n′} is independently hydrogen, hydroxy, CH₃C(O)—O, —OSO₃H, or —O-G; or
Rⁿ and the corresponding Rⁿ, where n is 2, 3, 4, 6, 7, or 8, taken together with the carbon to which they are attached, form C═O; or
Rⁿ along with the corresponding Rⁿ⁺¹, where n is 1, 2, 3, 4, 5, or 7, taken together form a double bond between the carbons to which they are attached, and each corresponding R^{n′} and R^{(n+1)′} is independently hydrogen, hydroxy, CH₃C(O)—O, —OSO₃H, or —O-G;
G has the formula: 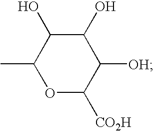
wherein the nitrogen denoted with the symbol * can optionally form an N-oxide;
X—Y is CH═N, CH═N(O), CH₂N(O), C(O)NH or CR⁹HNR¹⁰;
R⁹ is hydrogen, hydroxyl, or —OSO₃H;
R¹⁰ is hydrogen, acetyl, —SO₃H, -G, or —C(O)—OG;
Z is hydrogen, hydroxy, —OSO₃H, or —O-G;
with the proviso that when Z is hydroxy, then either (a) one of R¹, R², R³, R⁴, R⁵, R⁶, R⁷, R⁸, R⁹, and R¹⁰ is not hydrogen; or (b) X—Y is not CR⁹HNR¹⁰; and
with the further proviso that when X—Y is CHR⁹NR¹⁰, then at least one of Z, R¹, R², R³, R⁴, R⁵, R⁶, R⁷, R⁸, R⁹, and R¹⁰ is not H;
or a pharmaceutically acceptable salt thereof.

2. A compound or salt according to claim 1, wherein at least one of Z and R¹ through R⁸ is —OH.

3. A compound of claim 1 selected from the group consisting of: 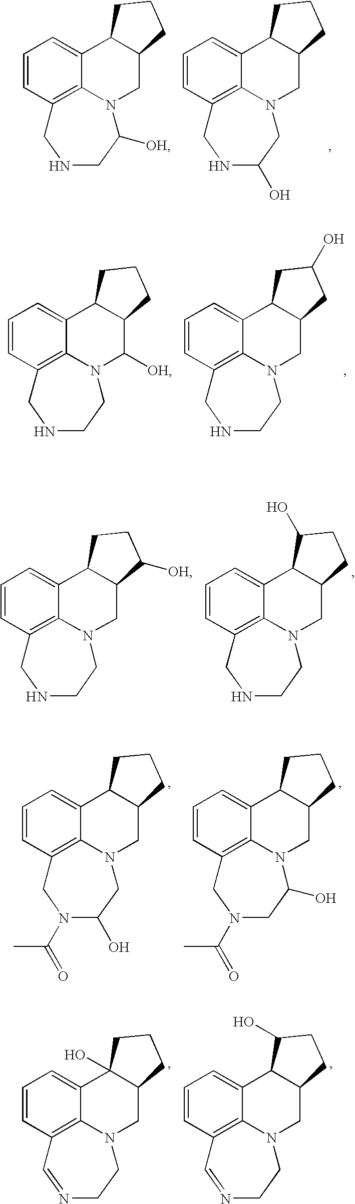 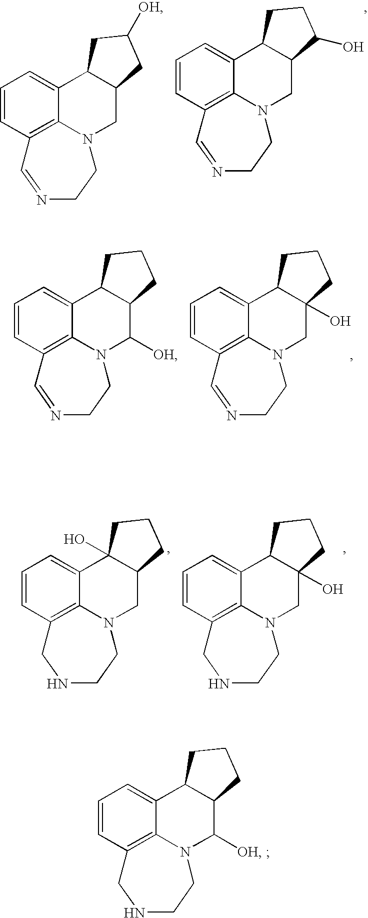
or a pharmaceutically acceptable salt thereof.

4. A compound or salt according to claim 1, wherein
at least one of R¹ through R⁶, R⁹, R¹⁰, and Z is —C(O)—O-G, —O-G, or -G.

5. A compound or salt according to claim 4, wherein X—Y is CR⁹HNR¹⁰.

6. A compound or salt according to claim 5, where R⁹ and R¹⁰ are H.

7. A compound or salt according to claim 5, wherein at least one of Z, R³, and R⁴is —O-G.

8. A compound or salt according to claim 5, wherein at least one of R¹ through R⁶, R⁹, and Z is —O-G.

9. A compound or salt according to claim 5, wherein R² along with R³ taken together form a double bond between the carbons to which they are attached, and at least one of R^{3′} and R⁴ is —O-G.

10. A compound or salt according to claim 5, wherein R⁴ and R^{4′} together with the carbon to which they are attached form C═O.

11. A compound or salt according to claim 10 wherein R¹⁰ is -G.

12. A compound or salt according to claim 5, where R¹⁰ is —C(O)O-G.

13. A compound or salt according to claim 5, where R¹⁰ is acetyl.

14. A compound or salt according to claim 13, wherein at least one of R¹ through R⁶, R⁹, and Z is —O-G.

16. A compound or salt according to claim 13, wherein at least one of R⁷ and R⁸ is —O-G.

17. A compound according to claim 1, wherein Z, each Rⁿ, and each R^{n′} is H; and X—Y is CR⁹HNR¹⁰.

18. A compound or salt according to claim 17, wherein R⁹ is H.

19. A compound or salt according to claim 17, wherein R⁹ is H and R¹⁰ is —C(O)—OG.

20. A compound of claim 1 selected from the group consisting of: 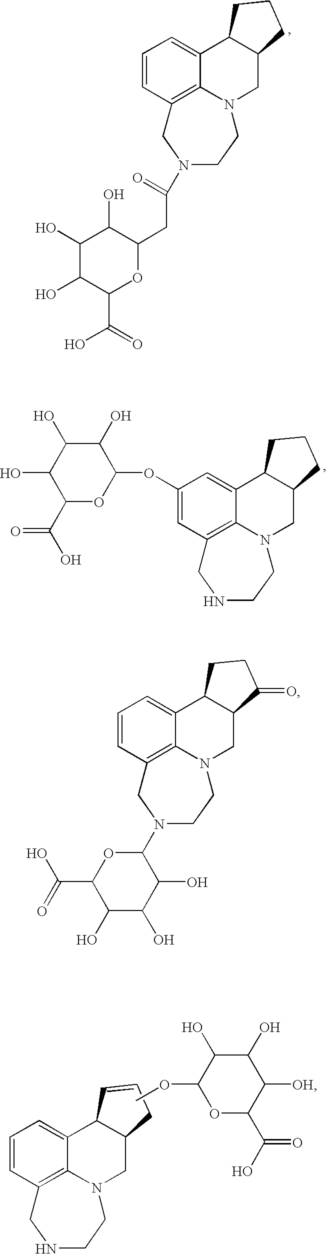 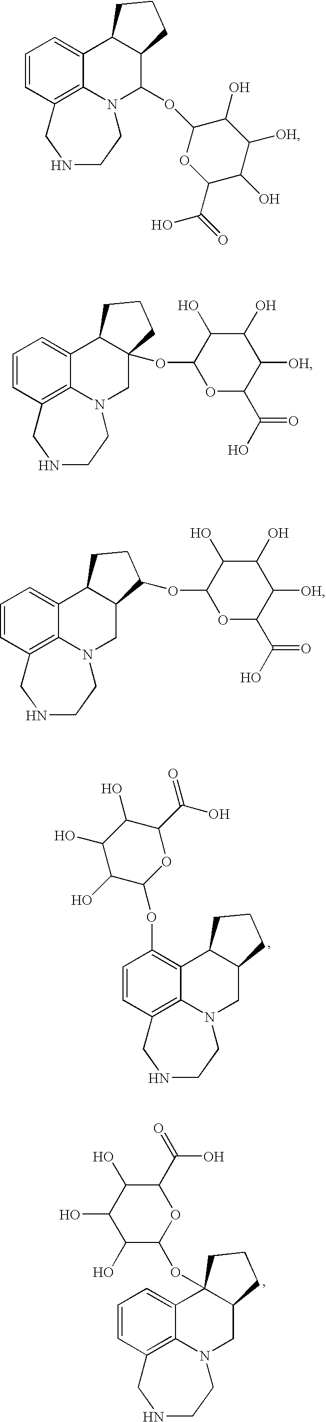 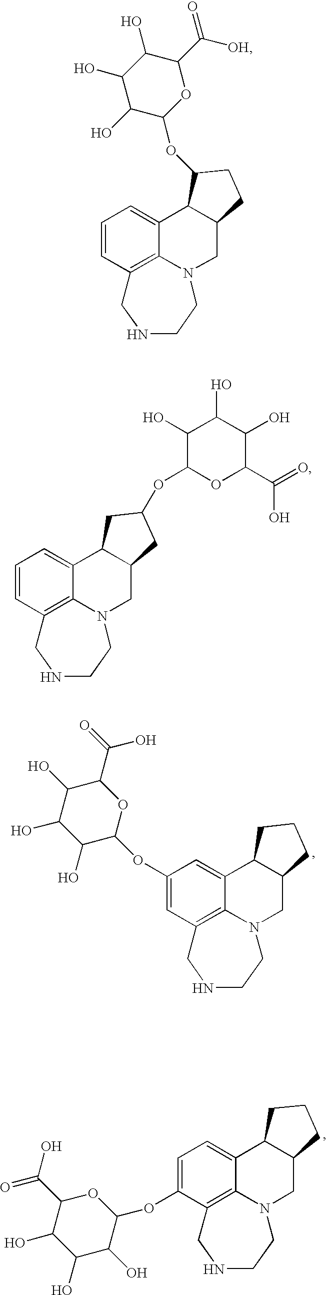 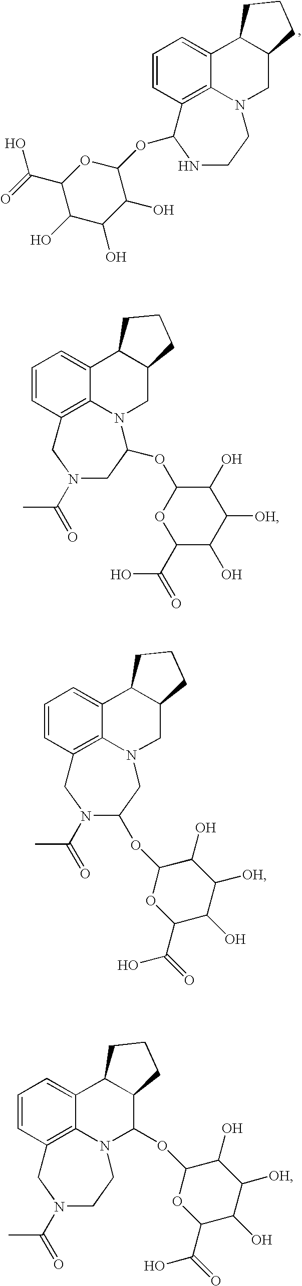 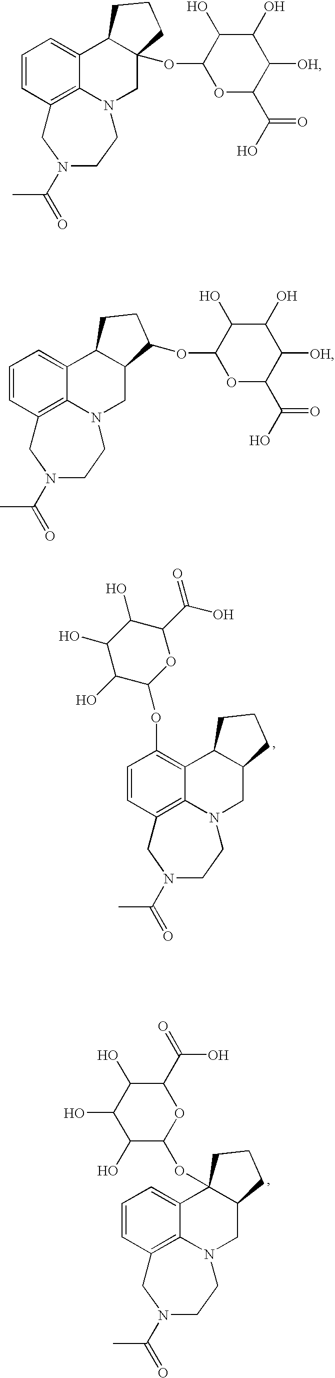 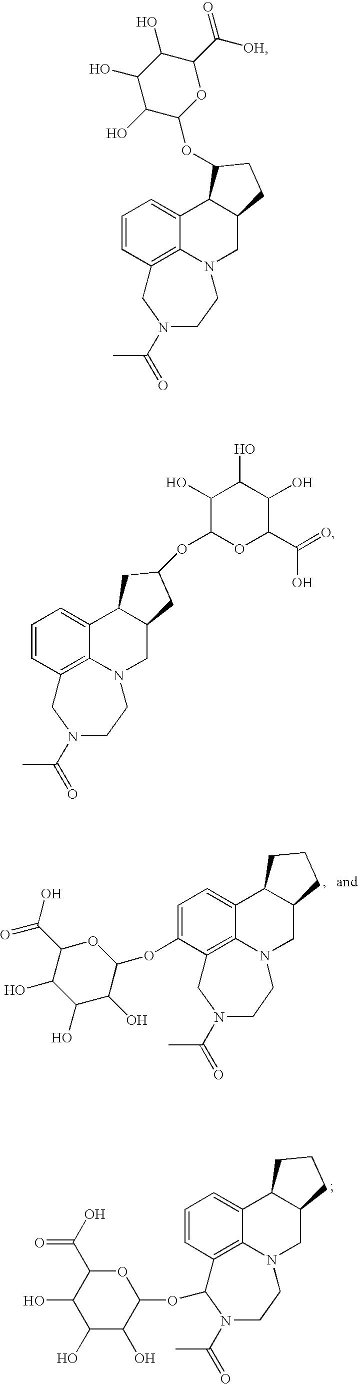
or a pharmaceutically acceptable salt thereof.

21. A compound or salt according to claim 1, wherein at least one of R¹ through R⁹, and Z is —OSO₃H.

22. A compound of claim 1 selected from the group consisting of: 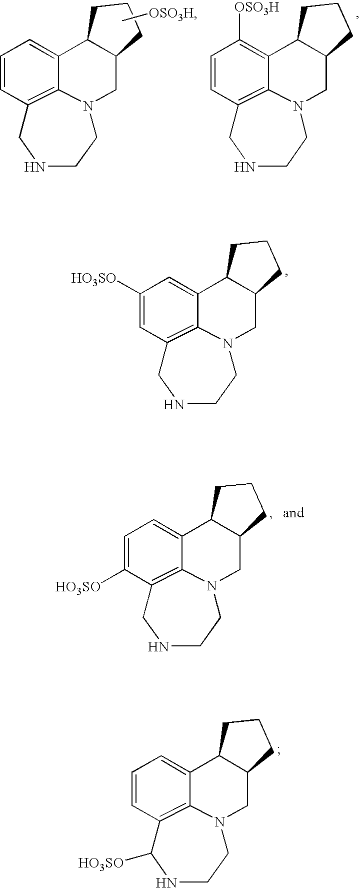
or a pharmaceutically acceptable salt thereof.

23. A compound or salt according to claim 1, wherein X—Y is CR⁹HNR¹⁰, where R⁹ is H and R¹⁰ is —SO₃H.

24. A compound of claim 1 selected from the group consisting of: 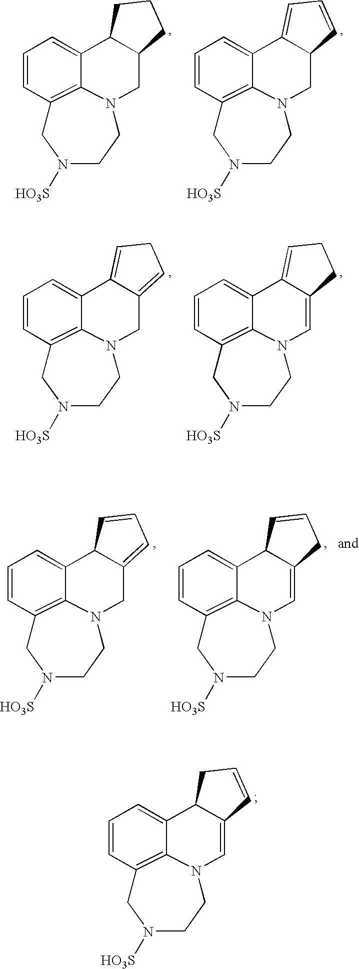
or a pharmaceutically acceptable salt thereof.

25. A compound or salt according to claim 1, wherein Rⁿ and corresponding R^{n′} taken together with the carbon to which they are attached form C═O.

26. A compound of claim 1 selected from the group consisting of: 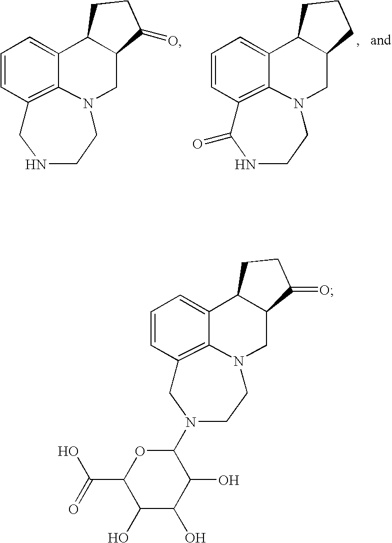
or a pharmaceutically acceptable salt thereof.

27. A compound or salt according to claim 1, wherein X—Y is C(O)NH.

28. A compound of claim 1 selected from the group consisting of: 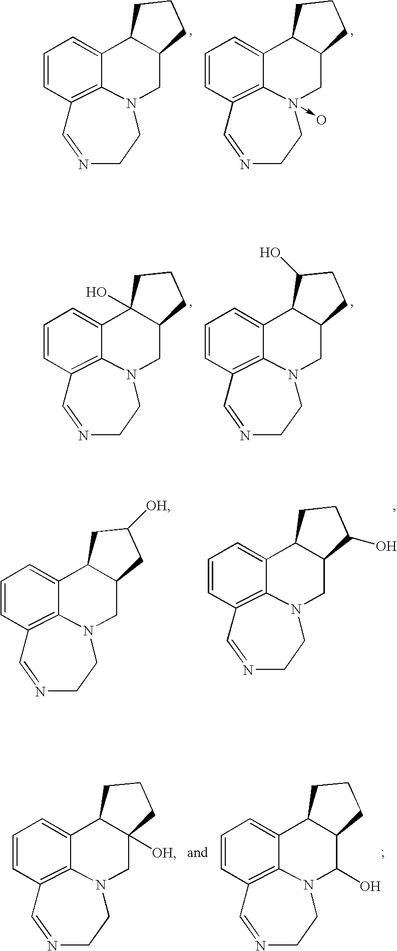
or a pharmaceutically acceptable salt thereof.

29. A compound or salt according to claim 1, wherein at least one of Rⁿ and its corresponding Rⁿ⁺¹, where n=1-5, together form a double bond between the carbons to which they are attached and each R^{n′} and R^{(n+1)′} is independently hydrogen, hydroxy, CH₃C(O)—O, —OSO₃H, or —O-G.

30. A compound or salt according to claim 29, wherein for at least two Rⁿ, each said Rⁿ and its corresponding Rⁿ⁺¹, where n=1-5, together form a double bond between the carbons to which they are attached.

31. A compound of claim 1 selected from the group consisting of: 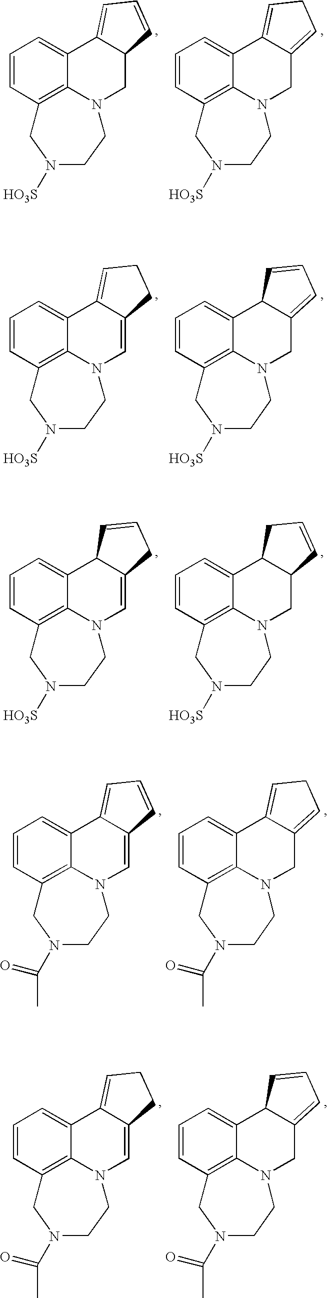 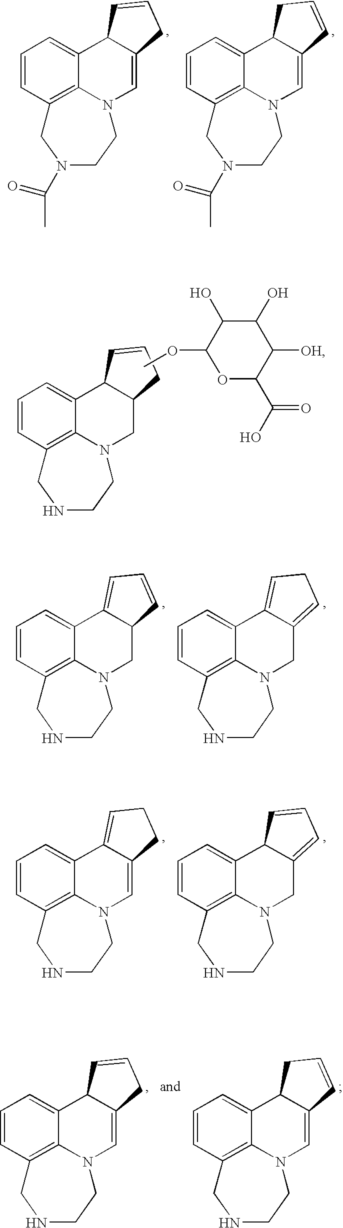
or a pharmaceutically acceptable salt thereof.

32. A pharmaceutical composition comprising a compound or salt of claim 1.

33. A method of preparing a compound of formula M6: 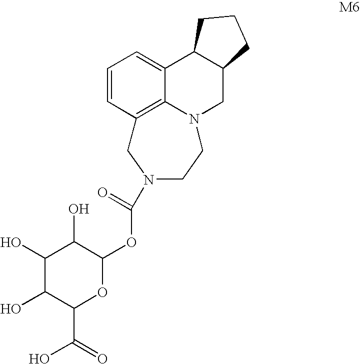
comprising:
reacting Compound 6a: 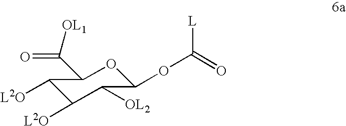
where each L, L¹, and L² is a leaving group;
with DCDQ: 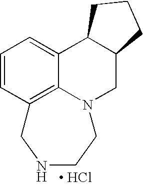
in the presence of a coupling reagent under conditions sufficient to yield compound 7: 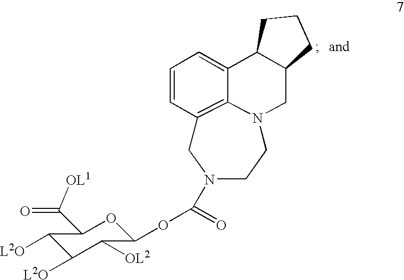
removing said leaving groups L¹ and L².

34. A method for treating a patient suffering from schizophrenia, schizophreniform disorder, schizoaffective disorder, delusional disorder, substance-induced psychotic disorder, L-DOPA-induced psychosis, psychosis associated with Alzheimer's dementia, psychosis associated with Parkinson's disease, psychosis associated with Lewy body disease, dementia, memory deficit, or intellectual deficit disorder associated with Alzheimer's disease comprising administering to the patient a therapeutically effective amount of a compound or salt according to claim 1 or a composition comprising such a compound or salt.

35. The method of claim 34, wherein the patient is suffering from schizophrenia.

36. A method for treating a patient suffering from bipolar disorders, depressive disorders, mood episodes, anxiety disorders, adjustment disorders, or eating disorders comprising administering to the patient a therapeutically effective amount of a compound or salt according to claim 1 or a composition comprising such a compound or salt.

37. The method of claim 36, wherein the bipolar disorder is bipolar I disorder, bipolar II disorder, or cyclothymic disorder; the depressive disorder is major depressive disorder, dysthymic disorder, or substance-induced mood disorder; the mood episode is major depressive episode, manic episode, mixed episode, or hypomanic episode; the anxiety disorder is panic attack, agoraphobia, panic disorder, specific phobia, social phobia, obsessive compulsive disorder, posttraumatic stress disorder, acute stress disorder, generalized anxiety disorder, separation anxiety disorder, or substance-induced anxiety disorder.

38. The method of claim 37, wherein the condition is depressive disorder, bipolar disorder or mood episode.

39. A method for treating a patient suffering from epilepsy, sleep disorders, migraines, sexual dysfunction, drug addiction, alcohol addiction, gastrointestinal disorders, or obesity comprising administering to the patient a therapeutically effective amount of a compound or salt according to claim 1 or a composition comprising such a compound or salt.

40. A method for treating a patient suffering from a central nervous system deficiency associated with trauma, stroke, or spinal cord injury comprising administering to the patient a therapeutically effective amount of a compound or salt according to claim 1 or a composition comprising such a compound or salt.

41. A composition comprising a compound or salt according to claim 1, and one or more pharmaceutically acceptable carriers.
